(19) United States
(12) Patent Application Publication
(10) Pub. No.: US 2007/0292385 A1
(43) Pub. Date: Dec. 20, 2007

(54) Phospholipase Inhibitors Localized in the Gastrointestinal Lumen

(76) Inventors: Dominique Charmot, Campbell, CA (US); Jerry M. Buysse, Los Altos, CA (US); Han-Ting Chang, Livermore, CA (US); Michael James Cope, Berkeley, CA (US); David Hui, Cincinnati, OH (US)

Correspondence Address: ILYPSA-McANDREWS;C/O MCANDREWS, HELD & MADISON STREET, 500 WEST MADISON STREET, SUITE 3400, CHICAGO, IL, 60661, US

(21) Appl. No.: 11/579,251

(22) PCT Filed: May 3, 2005
(86) PCT No.: PCT/US05/15418

§ 371 Date: May 25, 2007

(30) Foreign Application Priority Data
May 3, 2004 (US) 10838879

(51) Int. Cl. A61K 31/785; A61P 3/00

(52) U.S. Cl. 424/78.36

(57) ABSTRACT

The present invention provides methods and compositions for the treatment of phospholipase-related conditions. In particular, the invention provides a method of treating insulin-related, weight-related conditions and/or cholesterol-related conditions in an animal subject. The method generally involves the administration of a non-absorbed and/or effluxed phospholipase A2 inhibitor that is localized in a gastrointestinal lumen.

### RELATED APPLICATION

This application claims priority to co-owned, co-pending U.S. patent application Ser. No. 10/838,879 entitled “Phospholipase Inhibitors Localized in the Gastrointestinal Lumen” filed May 3, 2004 by Hui et al., which is incorporated by reference herein in its entirety.

### BACKGROUND OF THE INVENTION

Phospholipases are a group of enzymes that play important roles in a number of biochemical processes, including regulation of membrane fluidity and stability, digestion and metabolism of phospholipids, and production of intracellular messengers involved in inflammatory pathways, hemodynamic regulation and other cellular processes. Phospholipases are themselves regulated by a number of mechanisms, including selective phosphorylation, pH, and intracellular calcium levels. Phospholipase activities can be modulated to regulate their related biochemical processes, and a number of phospholipase inhibitors have been developed.

Certain phospholipase activities occur in the gastrointestinal lumen, for example, phospholipase A₂ acts in the digestion of dietary phospholipids in the gastrointestinal lumen, and phospholipase B is active in the apical mucosa of the distal intestine. The activities of these enzymes affect a number of phospholipase-related conditions, including diabetes, weight gain and cholesterol-related conditions.

Diabetes affects 18.2 million people in the United States, representing over 6% of the population. Diabetes is characterized by the inability to produce or properly use insulin. Diabetes type 2 (also called non-insulin-dependent diabetes or NIDDM) accounts for 80-90% of the diagnosed cases of diabetes and is caused by insulin resistance. Insulin resistance in diabetes type 2 prevents maintenance of blood glucose within desirable ranges, despite normal to elevated plasma levels of insulin.

Obesity is a major contributor to diabetes type 2, as well as other illnesses including coronary heart disease, osteoarthritis, respiratory problems, and certain cancers. Despite attempts to control weight gain, obesity remains a serious health concern in the United States and other industrialized countries. Indeed, over 60% of adults in the United States are considered overweight, with about 22% of these being classified as obese.

Diet also contributes to elevated plasma levels of cholesterol, including non-HDL cholesterol. Non-HDL cholesterol is associated with atherogenesis and its sequalea including arteriosclerosis, myocardial infarction, ischemic stroke, and other forms of heart disease that together rank as the most prevalent type of illness in industrialized countries. Indeed, an estimated 12 million people in the United States suffer with coronary artery disease and about 36 million require treatment for elevated cholesterol levels.

With the high prevalence of diabetes, obesity, and cholesterol-related conditions, there remains a need for approaches that treat one or more of these conditions, including reducing unwanted side effects. The present invention provides methods, compositions, and kits for using phospholipase inhibitors to treat phospholipase-related conditions, such as insulin-related conditions (e.g., diabetes), weight-related conditions (e.g., obesity) and/or cholesterol-related conditions.

Accordingly, there remains a need in the art for more beneficial phospholipase inhibitor compositions, methods of using such compositions, and treatments involving such compositions.

### SUMMARY OF THE INVENTION

One first aspect of the present invention relates to a composition comprising a phospholipase inhibitor. The phospholipase inhibitor is adapted such that (following administration to a subject) the phospholipase inhibitor is localized in a gastrointestinal lumen. In some embodiments included within a first general approach of this aspect of the invention, the inhibitor is not absorbed through a gastrointestinal mucosa. In embodiments included within a second general approach of this aspect of the invention, the inhibitor is localized in the gastrointestinal lumen as a result of efflux from a gastrointestinal mucosal cell.

Generally, in embodiments of the invention, including for example for embodiments relating to the aforementioned first general approach or second general approach, the inhibitor can have lumen-localization functionality. For example, the phospholipase inhibitor can have chemical and physical properties, such as low permeability (e.g., across biological membranes) that impart lumen-localization functionality to the inhibitor. Preferably, the inhibitors of these embodiments can additionally or alternatively have other chemical and/or physical properties such that at least about 80% of the phospholipase inhibitor remains in the gastrointestinal lumen, and preferably at least about 90% of the phospholipase inhibitor remains in the gastrointestinal lumen (in each case, following administration of the inhibitor to the subject). Such chemical and/or physical properties can be realized, for example, by an inhibitor comprising at least one moiety selected from an oligomer moiety, a polymer moiety, a hydrophobic moiety, a hydrophilic moiety, a charged moiety and combinations thereof. These embodiments can be used in various and specific combination, and in each permutation, with other aspects and embodiments described above or below herein.

Generally, in embodiments of the invention, including for example for embodiments relating to the first general approach or second general approach, the inhibitor can have enzyme-inhibiting functionality. For example, the phospholipase inhibitor can hinder access of a phospholipase to a phospholipid substrate. The oligomer moiety or polymer moiety can hinder access of a phospholipase to a phospholipid, for example by interacting with the phospholipase, or by interacting with the phospholipid substrate, or by interacting with both the phospholipase and the phospholipid. In some embodiments, the inhibitor can be effective for scavenging phospholipase, for example, within a fluid such as an aqueous phase of the gastrointestinal tract. In some embodiments, the inhibitor can be adapted to interact with a lipid-water interface, for example, of a lipid aggregate containing phospholipid substrate (e.g., a phospholipid-containing micelle or vesicle). In some embodiments, the inhibitor can interact with the phospholipase, for example with a specific site thereon, preferably with the catalytic site bearing face (e.g., the i-face) of a phospholipase such as phospholipid-A₂. In some embodiments, the inhibitor can interact (for example, with a specific site on a phospholipase, e.g., the catalytic site) reversibly or irreversibly. These embodiments can be used in various and specific combination, and in each permutation, with other aspects and embodiments described above or below herein.

Generally, in embodiments of the invention, including for example for embodiments relating to the first general approach or second general approach thereof, the phospholipase inhibitor can comprise or consist essentially of a small substituted organic molecule, an oligomer, a polymer, moieties of any thereof, and combinations of any of the foregoing. In some embodiments, the phospholipase inhibitor can comprise a phospholipase inhibiting moiety linked (e.g., covalently linked, directly or indirectly using a linking moiety) to a non-absorbed or non-absorbable moiety, preferably to a non-absorbed or non-absorbable oligomer or polymer moiety. In these embodiments, the phospholipase inhibiting moiety can be, for example, a moiety of a small substituted organic molecule having inhibiting functionality. These embodiments can be used in various and specific combination, and in each permutation, with other aspects and embodiments described above or below herein.

Generally, in embodiments comprising oligomers or polymers (or moieties thereof), the oligomers or polymers can be specifically configured and can be adapted to contribute to lumen-localization functionality and/or to enzyme-inhibiting functionality of the phospholipase inhibitor. The oligomer (or oligomer moiety) or the polymer (or polymer moiety): can generally be soluble or insoluble; can generally be a cross-linked oligomer (or oligomer moiety) or a cross-linked polymer (or polymer moiety); can generally be a homopolymer or a copolymer (including polymers having two monomer-repeat-units, terpolymers and higher-order polymers), including for example random copolymer moieties and block copolymer moieties; can generally include one or more ionic monomer moieties such as one or more anionic monomer moieties; can generally include one or more hydrophobic monomer moieties; can generally include one or more hydrophilic monomer moieties; and can generally include any of the foregoing features in combination. Particularly preferred embodiments of oligomers or polymers (or moieties thereof) are further described hereinafter in the context of independent aspects of the invention, but are equally applicable and are specifically contemplated as being applicable in conjunction with this first aspect of the invention, including both the first and second general approaches thereof. These embodiments can be used in various and specific combination, and in each permutation, with other aspects and embodiments described above or below herein.

Generally, in embodiments comprising a small substituted organic molecule (or a moiety thereof) as a phospholipase inhibitor (or as a phospholipase inhibiting moiety)—including embodiments with inhibitors comprising a phospholipase inhibiting moiety linked to a non-absorbed or non-absorbable moiety such as an oligomer or polymer moiety, the small molecule inhibitor or inhibiting moiety can be a known or future-discovered small molecule having phospholipase inhibiting activity. In some preferred embodiments, the small molecule phospholipase inhibitor or inhibiting moiety can comprise a moiety of a substituted organic compound having a fused five-member ring and six-member ring, and preferably a fused five-member ring and six-member ring having one or more heteroatoms (e.g., nitrogen, oxygen) substituted within the ring structure of the five-member ring, within the ring structure of the six-member ring, or within the ring structure of each of the five-member and six-member rings, and in each case with substituent groups effective for imparting phospholipase inhibiting functionality to the moiety. Preferably, such substituent groups are also effective for imparting lumen-localizing functionality to the moiety. In preferred embodiments, a small molecule phospholipase inhibitor or inhibiting moiety can comprise an indole-containing moiety (referred to herein interchangeably as an indole-moiety), such as a substituted indole moiety. In some embodiments, the phospholipase inhibitor or inhibiting moiety can be a phospholipid analog or a transition state analog. In some embodiments, the small molecule inhibitor or inhibiting moiety can further comprise at least one substituent having functionality for linking directly or indirectly to a non-absorbed or non-absorbable moiety, such as an oligomer or polymer moiety. For example, a phospholipids analog or transition state analog can be linked directly or indirectly to the non-absorbed moiety, for example, via its hydrophobic group. Particularly preferred embodiments of the phospholipase inhibitor or inhibiting moiety are further described hereinafter in the context of independent aspects of the invention, but are equally applicable and are specifically contemplated as being applicable in conjunction with this first aspect of the invention, including both the first and second general approaches thereof. Also, these embodiments can be used in various and specific combination, and in each permutation, with other aspects and embodiments described above or below herein.

Another second aspect of the invention relates to a composition comprising a phospholipase inhibitor, in which the phospholipase inhibitor comprises an oligomer or polymer moiety covalently linked to a phospholipase inhibiting moiety, and in which the phospholipase inhibitor is further characterized by one or more features selected from the group consisting of: (a) the phospholipase inhibitor being stable while passing through at least the stomach, the duodenum and the small intestine of the gastrointestinal tract; (b) the phospholipase inhibitor inhibiting activity of a secreted, calcium-dependent phospholipase present in the gastrointestinal lumen; (c) the phospholipase inhibitor inhibiting activity of a phosholipase-A₂ IB; (d) the phospholipase inhibitor inhibiting activity of a phosholipase-A₂, but essentially does not inhibit other gastrointestinal mucosal membrane-bound phospholipases; (e) the phospholipase inhibitor being insoluble in the fluid phase of the gastrointestinal tract; (f) the phospholipase inhibitor being adapted to associate with a lipid-water interface; (g) the oligomer or polymer moiety comprising at least one monomer that is anionic and at least one monomer that is hydrophobic; (h) the oligomer or polymer moiety being a copolymer moiety, the copolymer moiety being a random copolymer moiety, a block copolymer moiety; a hydrophobic copolymer moiety; and combinations thereof, and (i) combinations thereof, including each permutation of combinations. These features can also be characterizing features of embodiments within first aspect of the invention as described above. Reciprocally, the polymer moiety and/or the phospholipase inhibiting moiety of this second aspect of the invention can themselves be further characterized by features already described above in connection with the first aspect of the invention. These embodiments can be used in various and specific combination, and in each permutation, with other aspects and embodiments described above or below herein.

A further third aspect of the invention is directed to a composition comprising the phospholipase inhibitor, in which the phospholipase inhibitor comprises a repeat unit, an oligomer or a polymer having the formula (A) 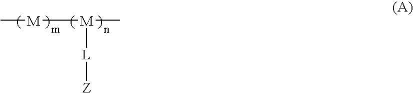
wherein n is an integer, m is an integer (with at least one of which m or n being a non-zero integer), M is a monomer moiety (i.e., a constituent moiety of a polymer) (e.g., each M being independently selected from one or more specific monomer moieties, such as a first monomer moiety, M₁, a second monomer moiety, M₂, a third monomer moiety M₃, a fourth monomer moiety, M₄, etc., where each thereof can be different from each other), L is an optional linking moiety and Z is a phospholipase inhibiting moiety. The phospholipase inhibitor preferably comprises an oligomer or a polymer having the formula (A). Embodiments included within this third aspect of the invention can be used in various and specific combination, and in each permutation, with other aspects and embodiments described above or below herein.

A fourth aspect of the invention is directed to a composition comprising the phospholipase inhibitor, where the phospholipase inhibitor comprises a compound of the formula (B) 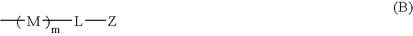
wherein m is a non-zero integer, M is a monomer moiety (e.g., each M being independently selected from one or more specific monomer moieties, such as a first monomer moiety, M₁, a second monomer moiety, M₂, a third monomer moiety M₃, a fourth monomer moiety, M₄, etc., where each thereof can be different from each other), L is an optional linking moiety and Z is a phospholipase inhibiting moiety. The embodiments included within this fourth aspect of the invention can be used in various and specific combination, and in each permutation, with other aspects and embodiments described above or below herein.

In a fifth aspect of the invention, a composition can comprise a phospholipase inhibitor, where the phospholipase inhibitor comprises a compound having the formula (C) 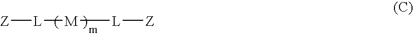
wherein m is a non-zero integer, M is a monomer moiety (e.g., each M being independently selected from one or more specific monomer moieties, such as a first monomer moiety, M₁, a second monomer moiety, M₂, a third monomer moiety M₃, a fourth monomer moiety, M₄, etc., where each thereof can be different from each other), L are each independently selected optional linking moieties and Z are each, independently selected phospholipase inhibiting moieties. Generally, these embodiments included within this fifth aspect of the invention can be used in various and specific combination, and in each permutation, with other aspects and embodiments described above or below herein.

In a sixth aspect, the invention is directed to a composition comprising a phospholipase inhibitor. The phospholipase inhibitor comprises an oligomer or polymer moiety covalently linked to a phospholipase inhibiting moiety, preferably with the phospholipase inhibitor comprising a compound having the formula (C-1) 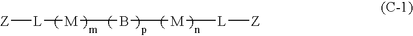
wherein m is a non-zero integer, n is a non-zero integer, p is a non-zero integer, M are each independently selected monomer moieties (e.g., each M being independently selected from one or more specific monomer moieties, such as a first monomer moiety, M₁, a second monomer moiety, M₂, a third monomer moiety M₃, a fourth monomer moiety, M₄, etc., where each thereof can be different from each other), B is a bridging moiety, L are each independently selected optional linking moieties, and Z are each independently selected phospholipase inhibiting moieties. Generally, these embodiments included within this sixth aspect of the invention can be used in various and specific combination, and in each permutation, with other embodiments described above or below herein.

In a seventh aspect, the invention relates to a composition comprising a phospholipase inhibitor, where the phospholipase inhibitor comprises an oligomer or polymer moiety covalently linked to a phospholipase inhibiting moiety. In this aspect, the oligomer or polymer moiety can have a repeat unit of formula (D) 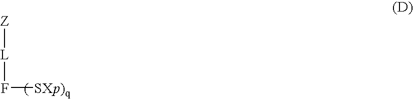
wherein Z is said phospholipase inhibiting moiety, L is a linking moiety; F is focal point where covalent linkages from a plurality of segments SXp converge; S is a spacer moiety; X is an anionic moiety, p is 1, 2, 3, or 4, and q is 2, 3, 4, 5, 6, 7, or 8. The embodiments included within this seventh aspect of the invention can be used in various and specific combination, and in each permutation, with other embodiments described above or below herein.

In each of the third, fourth, fifth, sixth and seventh aspects of the invention, the phospholipase inhibitor can be further characterized by one or more features selected from the features described above in connection with the first and/or second aspects of the invention. Further, the oligomer or polymer moieties of these third, fourth, fifth, sixth and seventh aspects of the invention can include those features already described above in connection with the various embodiments of the first and/or second aspects of the invention. Likewise, the phospholipase inhibiting moiety of these third, fourth, fifth, sixth and seventh aspects of the invention can comprise those features already described above in connection with the embodiments of the first and/or second aspects of the invention. These embodiments can be used in various and specific combination, and in each permutation, with other aspects and embodiments described above or below herein.

Generally, with respect to any of the aforementioned aspects or following-discussed aspects of the invention, the phospholipase inhibitor can be adapted so that it inhibits activity of a phospholipase, especially and preferably characterized in that the inhibitor: inhibits activity of a secreted, calcium-dependent phospholipase present in the gastrointestinal lumen; inhibits a phospholipase-A₂ present in the gastrointestinal lumen; inhibits activity of secreted, calcium-dependent phospholipase-A₂ present in the gastrointestinal lumen; inhibits activity of phospholipase-A₂ IB present in the gastrointestinal lumen; inhibits a phospholipase A₂, such as phospholipase-A₂ IB, as well as inhibits phospholipase B; and/or combinations thereof. These embodiments can be used in various and specific combination, and in each permutation, with other aspects and embodiments described above or below herein.

Also, with respect to any of the aspects of the invention, the phospholipase inhibitor can be relatively specific or strictly specific, for example, including having activity for inhibiting a phospholipase-A₂, such as a phospholipase-A₂ IB, but where the phospholipase inhibitor essentially does not inhibit one or more other enzymes, as follows: essentially does not inhibit a lipase; essentially does not inhibit phospholipase-B; essentially does not inhibit other gastrointestinal phospholipases having activity for catabolizing a phospholipids; essentially does not inhibit other gastrointestinal phospholipases having activity for catabolizing phosphatidylcholine or phosphatidylethanolamine; and/or essentially does not inhibit other gastrointestinal mucosal membrane-bound phospholipases, and combinations thereof. In some embodiments, the inhibitor does not act on the gastrointestinal mucosa. These embodiments can be used in various and specific combination, and in each permutation, with other aspects and embodiments described above or below herein.

Generally, in the embodiments included within any of the aspects of the invention, the phospholipase inhibitors herein can be characterized in that they produce a therapeutic and/or a prophylactic benefit in treating an insulin-related condition (e.g., diabetes type 2), a weight-related condition (e.g., obesity), a cholesterol-related condition (e.g., hypercholesterolemia), and combinations thereof, in each case in a subject receiving said inhibitor.

Another eighth aspect of the invention provides methods of using a composition comprising a phospholipase inhibitor (including, for example, any of the phospholipase inhibitors included within the first through seventh aspects of the invention). Generally, the method comprises inhibiting a phospholipase by administering an effective amount of the composition to a subject in need thereof. In some embodiments, the method comprises specifically or selectively inhibiting a phospholipase (e.g., with various aspects of specificity being as described above). These method embodiments can be used in various and specific combination, and in each permutation, with other aspects and embodiments described above or below herein.

In another ninth aspect, the invention is directed to method of treating a condition comprising administering an effective amount of a phospholipase inhibitor to a subject, and localizing the inhibitor in a gastrointestinal lumen such that upon administration to the subject, essentially all of the phospholipase inhibitor remains in the gastrointestinal lumen. In preferred embodiments, this ninth aspect of the invention can include, in one preferred approach, a method of treating a condition comprising administering an effective amount of a phospholipase-A₂ inhibitor to a subject, the phospholipase-A₂ inhibitor preferably being a phospholipase-A₂ IB inhibitor, and in any case, the phospholipase-A₂ inhibitor being localized in a gastrointestinal lumen upon administration to the subject. This aspect of the invention can also include, in a second preferred approach, a method for modulating the metabolism of fat, glucose or cholesterol in a subject, the method comprising administering an effective amount of a phospholipase-A₂ inhibitor to the subject, the phospholipase-A₂ inhibitor inhibiting activity of a secreted, calcium-dependent phospholipase-A₂ present in a gastrointestinal lumen, the phospholipase inhibitor being localized in the gastrointestinal lumen upon administration to the subject. Preferably, and generally, the embodiments of this method can include treating a condition by administering an effective amount of a phospholipase inhibitor to a subject in need thereof where the inhibitor is not absorbed through a gastrointestinal mucosa and/or where the inhibitor is localized in a gastrointestinal lumen as a result of efflux from a gastrointestinal mucosal cell. Such phospholipase inhibitors can be used in the treatment of phospholipase-related conditions, preferably phospholipase A₂-related conditions and phospholipase A₂-related conditions induced by diet. Preferably, the condition treated is an insulin-related condition (e.g., diabetes type 2), a weight-related condition (e.g., obesity), a cholesterol-related condition (e.g., hypercholesterolemia), and combinations thereof. These embodiments can be used in various and specific combination, and in each permutation, with other aspects and embodiments described above or below herein.

In a related tenth aspect, the invention is directed to medicament comprising a phospholipase-A₂ inhibitor for use as a pharmaceutical. The phospholipase-A₂ inhibitor of the medicament is localized in a gastrointestinal lumen upon administration of the medicament to a subject. Preferably, the medicament comprises a phospholipase-A₂ IB inhibitor. These embodiments can be used in various and specific combination, and in each permutation, with other aspects and embodiments described above or below herein.

In another eleventh aspect, the invention is directed to a method comprising use of a phospholipase-A₂ inhibitor for manufacture of a medicament for use as a pharmaceutical, where the phospholipase-A₂ inhibitor is localized in a gastrointestinal lumen upon administration of the medicament to a subject. Preferably, the medicament is manufactured using a phospholipase-A₂ IB inhibitor. These embodiments can be used in various and specific combination, and in each permutation, with other aspects and embodiments described above or below herein.

A further twelfth aspect of the invention is directed to a food product composition comprising an edible foodstuff and a phospholipase inhibitor (such as a phospholipase-A₂ inhibitor) where the phospholipase inhibitor (or phospholipase-A₂ inhibitor) is localized in a gastrointestinal lumen upon ingestion of the food product composition. Preferably, the foodstuff comprises a phospholipase-A₂ IB inhibitor. In some embodiments, the foodstuff can comprise (or can consist essentially of) a vitamin supplement and a phospholipase inhibitor. These embodiments can be used in various and specific combination, and in each permutation, with other aspects and embodiments described above or below herein.

Generally, and preferably in connection with any of the eighth through twelfth aspects of the invention, the phospholipase-A₂ inhibitor does not induce substantial steatorrhea following administration or ingestion thereof. These embodiments can be used in various and specific combination, and in each permutation, with other aspects and embodiments described above or below herein.

Yet another thirteenth aspect of the invention relates to a method of making or identifying a phospholipase inhibitor that is localized in a gastrointestinal lumen by contacting a candidate moiety with a phospholipase A₂, a lipid-water interface, phospholipase B, or fragment thereof; determining whether the candidate moiety interacts with the phospholipase A₂, interface, phospholipase B, or fragment thereof; selecting said candidate moiety that interacts with phospholipase A₂, interface, phospholipase B, or fragment thereof; and using the selected candidate moiety as a phospholipase A₂ or phospholipase B inhibiting moiety of the phospholipase inhibitor that is localized in the gastrointestinal lumen. In some embodiment, a candidate moiety is selected that does not interact with phospholipase B or fragment thereof. These embodiments can be used in various and specific combination, and in each permutation, with other aspects and embodiments described above or below herein.

Those of skill in the art will recognize that the compounds described herein may exhibit the phenomena of tautomerism, conformational isomerism, geometric isomerism and/or optical isomerism. It should be understood that the invention encompasses any tautomeric, conformational isomeric, optical isomeric and/or geometric isomeric forms of the compounds having one or more of the utilities described herein, as well as mixtures of these various different forms. Prodrugs and active metabolites of the compounds described herein are also within the scope of the present invention.

Although various features are described above to provide a summary of various aspects of the invention, it is contemplated that many of the details thereof as described below can be used with each of the various aspects of the invention, without limitation. Other features, objects and advantages of the present invention will be in part apparent to those skilled in art and in part pointed out hereinafter. All references cited in the instant specification are incorporated by reference for all purposes. Moreover, as the patent and non-patent literature relating to the subject matter disclosed and/or claimed herein is substantial, many relevant references are available to a skilled artisan that will provide further instruction with respect to such subject matter.

### BRIEF DESCRIPTION OF THE DRAWINGS

FIG. 1A through FIG. 1D are schematic representations illustrating: (i) interaction of a phospholipase with a lipid-water interface (FIG. 1A); (ii) interaction of a non-absorbed phospholipase inhibitor with a lipid-water interface (FIG. 1B); (iii) interaction of a non-absorbed phospholipase inhibitor with the phospholipase enzyme (FIG. 1C); and (iv) interaction of a non-absorbed phospholipase inhibitor with both a lipid-water interface and with the phospholipase enzyme (FIG. 1D).

FIG. 2 is a schematic representation illustrating phospholipase inhibitors comprising polymer moieties covalently linked to phospholipase inhibiting moieties (represented schematically by “I*”), where the polymer moieties are shown as being soluble or insoluble, and further illustrating interaction between the phospholipase inhibitors and phospholipase-A₂ in a gastrointestinal fluid in the vicinity of gastrointestinal lipid vesicles.

FIG. 3A through FIG. 3C are schematic representations illustrating phospholipase inhibitors comprising polymer moieties covalently linked to one or more phospholipase inhibiting moiety (represented schematically by “I*”), where (i) the phospholipase inhibitor comprises a hydrophobic polymer moiety, adapted such that the inhibitor associates with a lipid-water interface of a lipid vesicle (shown with the hydrophobic polymer moiety being substantially integral with the lipid bilayer) (FIG. 3A); (ii) the phospholipase inhibitor comprises a polymer moiety having a first hydrophobic block and a second hydrophilic block with the second hydrophilic block being proximal to the phospholipase inhibiting moiety, adapted such that the inhibitor associates with a lipid-water interface of a lipid vesicle (shown with the hydrophobic block being substantially integral with the lipid bilayer and with the hydrophilic block being substantially associated within the aqueous phase surrounding the lipid bilayer) (FIG. 3B); and (iii) the phospholipase inhibitor comprises a hydrophobic polymer moiety covalently linked to two inhibiting moieties, and adapted such that the inhibitor associates with a lipid-water interface of a lipid vesicle (shown with the hydrophobic polymer moiety being substantially integral with and looped through the lipid bilayer (FIG. 3C); and in each case (i), (ii) and (iii) allowing for interaction between the inhibiting moiety and phospholipase-A₂ substantially proximate to the vesicle surface.

FIG. 4 is a schematic representation of a chemical reaction in which phospholipase-A2 enzyme (PLA2) catalyzes hydrolysis of phospholipids to corresponding lysophospholipids.

FIG. 5 is a chemical formula for [2-(3-(2-amino-2-oxoacetyl)-1-(biphenyl-2-ylmethyl)-2-methyl-1H-indol-4-yloxy)acetic acid], also referred to herein as ILY-4001 and as methyl indoxam.

FIGS. 6A through 6D are schematic representations including chemical formulas illustrating indole compounds (FIG. 6A, FIG. 6C and FIG. 6D) and indole-related compounds (FIG. 6B).

FIG. 7 is a schematic illustration, including chemical formulas, which outlines the overall synthesis scheme for ILY-4001 [2-(3-(2-amino-2-oxoacetyl)-1-(biphenyl-2-ylmethyl)-2-methyl-1H-indol-4-yloxy)acetic acid] as described in Example 1A.

FIGS. 8A and 8B are a schematic representation (FIG. 8A) of an in-vitro fluorometric assay for evaluating PLA2 IB enzyme inhibition, and a graph (FIG. 8B) showing the results of Example 6A in which the assay was used to evaluate ILY-4001 [2-(3-(2-amino-2-oxoacetyl)-1-(biphenyl-2-ylmethyl)-2-methyl-1H-indol-4-yloxy)acetic acid].

FIGS. 9A and 9B are graphs showing the results from the in-vitro Caco-2 permeability study of Example 6B for ILY-4001 [2-(3-(2-amino-2-oxoacetyl)-1-(biphenyl-2-ylmethyl)-2-methyl-1H-indol-4-yloxy)acetic acid] (FIG. 9A) and for Lucifer Yellow and Propranolol as paracellular and transcellular transport controls (FIG. 9B).

FIG. 10 is a schematic illustration, including chemical formulas, which outlines the overall synthesis scheme to prepare 3-(3-aminooxalyl-1-biphenyl-2-yl methyl-4-carboxymethoxy-2-methyl-1H-indol-5-yl)-propionic acid as described in Example 1C.

FIG. 11 is a schematic illustration, including chemical formulas, which outlines the overall synthesis scheme for preparing a polymer-linked ILY-4001—namely, a random copolymer of [3-Aminooxalyl-2-methyl-1-(2′-vinyl-biphenyl-2-ylmethyl)-1H-indol-4-yloxy]-acetic acid, styrene, and styrene sulfonic acid sodium salt, as described in Example 1D.

FIG. 12 is a schematic illustration, including chemical formulas, which outlines the overall synthesis scheme by which ILY-4001 can be provided with linking groups to form [3-Aminooxalyl-2-methyl-1-(4-vinyl-benzyl)-1H-indol-4-yloxy]-acetic acid (21); Synthesis of (1-Acryloyl-3-aminooxalyl-2-methyl-1H-indol-4-yloxy)-acetic acid (23); Synthesis of {3-Aminooxalyl-2-methyl-1-[2-(pyrazole-1-carbothioylsulfanyl)propionyl]-1H-indol-4-yloxy}-acetic acid (26), as described in Example 2.

FIGS. 13A through 13D are graphs summarizing the results of an in-vivo study of Example 10, including: a graph illustrating the results of Example 10A, showing body weight gain in groups of mice receiving ILY-4001 at low dose (4001-L) and high dose (4001-H) as compared to wild-type control group (Control) and as compared to genetically deficient PLA2 (−/−) knock-out mice (PLA2 KO) (FIG. 13A); a graph illustrating the results of Example 10B, showing fasting serum glucose levels in groups of mice receiving ILY-4001 at low dose (4001-L) and high dose (4001-H) as compared to wild-type control group (Control) and as compared to genetically deficient PLA2 (−/−) knock-out mice (PLA2 KO) (FIG. 13B); and graphs illustrating the results of Example 10C, showing serum cholesterol levels (FIG. 13C) and serum triglyceride levels (FIG. 13D) in groups of mice receiving ILY-4001 at low dose (4001-L) and high dose (4001-H) as compared to wild-type control group (Control) and as compared to genetically deficient PLA2 (−/−) knock-out mice (PLA2 KO).

### DETAILED DESCRIPTION OF THE INVENTION

The present invention provides phospholipase inhibitors, compositions (including pharmaceutical formulations, medicaments and foodstuffs) comprising such phospholipase inhibitors, and methods for identifying, making and using such phospholipase inhibitors and compositions, including use thereof as pharmaceuticals for treatments of various conditions. The phospholipase inhibitors of the present invention can find use in treating a number of phospholipase-related conditions, including insulin-related conditions (e.g., diabetes), weight-related conditions (e.g., obesity), cholesterol-related disorders and any combination thereof, as described in detail below.

Generally, the phospholipase inhibitors of the invention should be adapted for having both lumen-localization functionality as well as enzyme-inhibition functionalization. In some schema, certain aspects of such dual functionality can be achieved synergistically (e.g., by using the same structural features and/or charge features); in other schema, the lumen-localization functionality can be achieved independently (e.g., using different structural and/or charge features) from the enzyme-inhibition functionality.

Overview

The phospholipase inhibitors are preferably localized in the gastrointestinal lumen, such that upon administration to a subject, the phospholipase inhibitors remain substantially in the gastrointestinal lumen. Following administration, the localized phospholipase inhibitors can remain in and pass naturally through the gastrointestinal tract, including the stomach, the duodenum, the small intestine and the large intestine (until passed out of the body via the gastrointestinal tract). The phospholipase inhibitors are preferably substantially stable (e.g., with respect to composition and/or with respect to functionality for inhibiting phospholipase) while passing through at least the stomach and the duodenum, and more preferably, are substantially stable while passing through the stomach, the duodenum and the small intestine of the gastrointestinal tract, and most preferably, are substantially stable while passing through the entire gastrointestinal tract. The phospholipase inhibitors can act in the gastrointestinal lumen, for example to catabolize phospholipase substrates or to modulate the absorption and/or downstream activities of products of phospholipase digestion.

In the present invention, phospholipase inhibitors are localized within the gastrointestinal lumen, in one approach, by being not absorbed through a gastrointestinal mucosa. In some embodiments, the phospholipase inhibitors of the present invention can be localized in a gastrointestinal lumen and can also be cell impermeable, e.g., not internalized into a cell. As another approach, the phospholipase inhibitors can be localized in the gastrointestinal lumen by being absorbed into a mucosal cell and then effluxed back into a gastrointestinal lumen. Hence, in some embodiments, the phospholipase inhibitors are cell permeable, e.g., can be internalized into a cell, and are also localized in a gastrointestinal lumen. In these embodiments, gastrointestinal localization can be facilitated by an efflux mechanism. Each of these general approaches for achieving gastrointestinal localization is further described below.

Generally, without being constrained by categorization into one or more of the aforementioned general approaches by which the phospholipase inhibitor can be lumen-localized, preferred phospholipase inhibitors of the invention (as contemplated in the various aspects of the invention) can be realized by several general embodiment formats. In one general embodiment, for example, the phospholipase inhibitor can consist essentially of an oligomer or a polymer. In another embodiment, the phospholipase inhibitor can comprise an oligomer or polymer moiety covalently linked, directly or indirectly through a linking moiety, to a phospholipase inhibiting moiety, such as a substituted small organic molecule moiety. In a further general embodiment, the phospholipase inhibitor can itself be a substituted small organic molecule. Each of these general embodiments is described below in further detail.

In general for each various embodiments included within the various aspects of the invention, the inhibitor is localized, upon administration to a subject, in the gastrointestinal lumen of the subject, such as an animal, and preferably as a mammal, including for example a human as well as other mammals (e.g., mice, rats, rabbits, guinea pigs, hamsters, cats, dogs, porcine, poultry, bovine and horses). The term “gastrointestinal lumen” is used interchangeably herein with the term “lumen,” to refer to the space or cavity within a gastrointestinal tract, which can also be referred to as the gut of the animal. In some embodiments, the phospholipase inhibitor is not absorbed through a gastrointestinal mucosa. “Gastrointestinal mucosa” refers to the layer(s) of cells separating the gastrointestinal lumen from the rest of the body and includes gastric and intestinal mucosa, such as the mucosa of the small intestine. In some embodiments, lumen localization is achieved by efflux into the gastrointestinal lumen upon uptake of the inhibitor by a gastrointestinal mucosal cell. A “gastrointestinal mucosal cell” as used herein refers to any cell of the gastrointestinal mucosa, including, for example, an epithelial cell of the gut, such as an intestinal enterocyte, a colonic enterocyte, an apical enterocyte, and the like. Such efflux achieves a net effect of non-absorbedness, as the terms, related terms and grammatical variations, are used herein.

Generally, in all embodiments included within the various aspects of the invention, phospholipase inhibitors of the present invention can modulate or inhibit (e.g., blunt or reduce) the catalytic activity of phospholipases, preferably phospholipases secreted or contained in the gastrointestinal tract, including the gastric compartment, and more particularly the duodenum and/or the small intestine. For example, such enzymes include, but are not limited to, secreted Group IB phospholipase A₂ (PL A₂-IB), also referred to as pancreatic phospholipase A₂ (p-PL A₂) and herein referred to as “PL A₂ IB” or “phospholipase-A₂ IB;” secreted Group IIA phospholipase A₂ (PL A₂ IIA); phospholipase A1 (PLA₁); phospholipase B (PLB); phospholipase C (PLC); and phospholipase D (PLD). The inhibitors of the invention preferably inhibit the activity at least the phospholipase-A₂ IB enzyme.

In some embodiments, the inhibitors of the present invention are specific, or substantially specific for inhibiting phospholipase activity, such as phospholipase A₂ activity (including for example phospholipase-A₂ IB). For example, in some preferred embodiments inhibitors of the present invention do not inhibit or do not significantly inhibit or essentially do not inhibit lipases, such as pancreatic triglyceride lipase (PTL) and carboxyl ester lipase (CEL). In some preferred embodiments, inhibitors of the present invention inhibit PL A₂, and preferably phospholipase-A₂ IB, but in each case do not inhibit or do not significantly inhibit or essentially do not inhibit any other phospholipases; in some preferred embodiments, inhibitors of the present invention inhibit PL A₂, and preferably phospholipase-A₂ IB, but in each case do not inhibit or do not significantly inhibit or essentially do not inhibit PLA₁; in some preferred embodiments, inhibitors of the present invention inhibit PL A₂, and preferably phospholipase-A₂ IB, but do not inhibit or do not significantly inhibit or essentially do not inhibit PLB. In some embodiments, the phospholipase inhibitor does not act on the gastrointestinal mucosa, for example, it does not inhibit or does not significantly inhibit or essentially does not inhibit membrane-bound phospholipases.

The different activities of PL A₂, PL A₁, and PLB are generally well-characterized and understood in the art. PL A₂ hydrolyzes phospholipids at the sn-2 position liberating 1-acyl lysophospholipids and fatty acids; PL A₁ acts on phospholipids at the sn-1 position to release 2-acyl lysophospholipids and fatty acids; and phospholipase B cleaves phospholipids at both sn-1 and sn-2 positions to form a glycerol and two fatty acids. See, e.g., Devlin, Editor, Textbook of Biochemistry with Clinical Correlations, 5^{th} ed. Pp 1104-1110 (2002).

Phospholipids substrates acted upon by gastrointestinal PL A₁, PL A₂ (including phospholipase-A₂ IB) and PLB are mostly of the phosphatidylcholine and phosphatidylethanolamine types, and can be of dietary or biliary origin, or may be derived from being sloughed off of cell membranes. For example, in the case of phosphatidylcholine digestion, PL A₁ acts at the sn-1 position to produce 2-acyl lysophosphatidylcholine and free fatty acid; PL A₂ acts at the sn-2 position to produce 1-acyl lysophosphatidylcholine and free fatty acid; while PLB acts at both positions to produce glycerol 3-phosphorylcholine and two free fatty acids (Devlin, 2002).

Pancreatic PL A₂ (and phospholipase-A₂ IB) is secreted by acinar cells of the exocrine pancreas for release in the duodenum via pancreatic juice. PL A₂ (and phospholipase-A₂ IB) is secreted as a proenzyme, carrying a polypeptide chain that is subsequently cleaved by proteases to activate the enzyme's catalytic site. Documented structure-activity-relationships (SAR) for PL A₂ isozymes illustrate a number of common features (see for instance, Gelb M., Chemical Reviews, 2001, 101:2613-2653; Homan, R., Advances in Pharmacology, 1995, 12:31-66; and Jain, M. K., Intestinal Lipid Metabolism, Biology, pathology, and interfacial enzymology of pancreatic phospholipase A₂, 2001, 81-104, each incorporated herein by reference).

The inhibitors of the present invention can take advantage of certain of these common features to inhibit phospholipase activity and especially PL A₂ activity. Common features of PL A₂ enzymes include sizes of about 13 to about 15 kDa; stability to heat; and 6 to 8 disulfides bridges. Common features of PL A₂ enzymes also include conserved active site architecture and calcium-dependent activities, as well as a catalytic mechanism involving concerted binding of His and Asp residues to water molecules and a calcium cation, in a His-calcium-Asp triad. A phospholipid substrate can access the catalytic site by its polar head group through a slot enveloped by hydrophobic and cationic residues (including lysine and arginine residues) described in more detail below. Within the catalytic site, the multi-coordinated calcium ion activates the acyl carbonyl group of the sn-2 position of the phospholipid substrate to bring about hydrolysis (Devlin, 2002). In some preferred embodiments, inhibitors of the present invention inhibit this catalytic activity of PL A₂ by interacting with its catalytic site.

PL A₂ enzymes are active for catabolizing phospholipids substrates primarily at the lipid-water interface of lipid aggregates found in the gastrointestinal lumen, including, for example, fat globules, emulsion droplets, vesicles, mixed micelles, and/or disks, any one of which may contain triglycerides, fatty acids, bile acids, phospholipids, phosphatidylcholine, lysophospholipids, lysophosphatidylcholine, cholesterol, cholesterol esters, other amphiphiles and/or other diet metabolites. Such enzymes can be considered to act while “docked” to a lipid-water interface. In such lipid aggregates, the phospholipid substrates are typically arranged in a mono layer or in a bilayer, together with one or more other components listed above, which form part of the outer surface of the aggregate. The surface of a phospholipase bearing the catalytic site contacts this interface facilitating access to phospholipid substrates. This surface of the phospholipase is known as the i-face, i.e., the interfacial recognition face of the enzyme. The structural features of the i-face of PL A₂ have been well documented. See, e.g., Jain, M. K, et al, Methods in Enzymology, vol. 239, 1995, 568-614, incorporated herein by reference. The inhibitors of the present invention can take advantage of these structural features to inhibit PL A₂ activity. For instance, it is known that the aperture of the slot forming the catalytic site is normal to the i-face plane. The aperture is surrounded by a first crown of hydrophobic residues (mainly leucine and isoleucine residues), which itself is contained in a ring of cationic residues (including lysine and arginine residues). In some preferred embodiments, inhibitors of the present invention hinder access of PL A₂ to its phospholipid substrates by interacting with this i-face and/or with the lipid-water interface.

In view of the action of phospholipases (e.g. PL A₂) in digesting phospholipid substrates in proximity to the surface of such lipid-aggregates, some embodiments of the invention can involve an approach in which the phospholipase inhibitor associates with a water-lipid interface of a lipid aggregate, thereby allowing for interaction between the inhibitor and phospholipase-A₂ substantially proximal thereto.

Localization within the Gastrointestinal Lumen Via Non-Absorbtion

In preferred approaches, the phosphate inhibitor can be an inhibitor that is substantially not absorbed from the gastrointestinal lumen into gastrointestinal mucosal cells. As such, “not absorbed” as used herein can refer to inhibitors adapted such that a significant amount, preferably a statistically significant amount, more preferably essentially all of the phospholipase inhibitor, remains in the gastrointestinal lumen. For example, at least about 80% of phospholipase inhibitor remains in the gastrointestinal lumen, at least about 85% of phospholipase inhibitor remains in the gastrointestinal lumen, at least about 90% of phospholipase inhibitor remains in the gastrointestinal lumen, at least about 95%, at least about 98%, preferably at least about 99%, and more preferably at least about 99.5% remains in the gastrointestinal lumen (in each case based on a statistically relevant data set). Reciprocally, stated in terms of serum bioavailability, a physiologically insignificant amount of the phospholipase inhibitor is absorbed into the blood serum of the subject following administration to a subject. For example, upon administration of the phospholipase inhibitor to a subject, not more than about 20% of the administered amount of phospholipase inhibitor is in the serum of the subject (e.g., based on detectable serum bioavailability following administration), preferably not more than about 15% of phospholipase inhibitor, and most preferably not more than about 10% of phospholipase inhibitor is in the serum of the subject. In some embodiments, not more than about 5%, not more than about 2%, preferably not more than about 1%, and more preferably not more than about 0.5% is in the serum of the subject (in each case based on a statistically relevant data set). In some cases, localization to the gastrointestinal lumen can refer to reducing net movement across a gastrointestinal mucosa, for example, by way of both transcellular and paracellular transport, as well as by active and/or passive transport. The phospholipase inhibitor in such embodiments is hindered from net permeation of a gastrointestinal mucosal cell in transcellular transport, for example, through an apical cell of the small intestine; the phospholipase inhibitor in these embodiments is also hindered from net permeation through the “tight junctions” in paracellular transport between gastrointestinal mucosal cells lining the lumen. The term “not absorbed” is used interchangeably herein with the terms “non-absorbed,” “non-absorbedness,” “non-absorption” and its other grammatical variations.

In some embodiments, detailed further below, an inhibitor or inhibiting moiety can be adapted to be non-absorbed by modifying the charge and/or size, particularly, as well as additionally other physical or chemical parameters of the phospholipase inhibitor. For example, in some embodiments, the phospholipase inhibitor is constructed to have a molecular structure that minimizes or nullifies absorption through a gastrointestinal mucosa. The absorption character of a drug can be selected by applying principles of pharmacodynamics, for example, by applying Lipinsky's rule, also known as “the rule of five.” As a set of guidelines, Lipinsky shows that small molecule drugs with (i) molecular weight, (ii) number of hydrogen bond donors, (iii) number of hydrogen bond acceptors, and (iv) water/octanol partition coefficient (Moriguchi logP) each greater than a certain threshold value generally do not show significant systemic concentration. See Lipinsky et al, Advanced Drug Delivery Reviews, 46, 2001 3-26, incorporated herein by reference. Accordingly, non-absorbed phospholipase inhibitors can be constructed to have molecule structures exceeding one or more of Lipinsky's threshold values, and preferably two or more, or three or more, or four or more or each of Lipinsky's threshold values. See also Lipinski et al., Experimental and computational approaches to estimate solubility and permeability in drug discovery and development settings, Adv. Drug Delivery Reviews, 46:3-26 (2001); and Lipinski, Drug-like properties and the causes of poor solubility and poor permeability, J. Pharm. & Toxicol. Methods, 44:235-249 (2000), incorporated herein by reference. In some preferred embodiments, for example, a phospholipase inhibitor of the present invention can be constructed to feature one or more of the following characteristics: (i) having a MW greater than about 500 Da; (ii) having a total number of NH and/or OH and/or other potential hydrogen bond donors greater than about 5; (iii) having a total number of O atoms and/or N atoms and/or other potential hydrogen bond acceptors greater than about 10; and/or (iv) having a Moriguchi partition coefficient greater than about 10⁵, i.e., logP greater than about 5. Any art known phospholipase inhibitors and/or any phospholipase inhibiting moieties described below can be used in constructing a non-absorbed molecular structure.

Preferably, the permeability properties of the compounds are screened experimentally: permeability coefficient can be determined by methods known to those of skill in the art, including for example by Caco-2 cell permeability assay. The human colon adenocarcinoma cell line, Caco-2, can be used to model intestinal drug absorption and to rank compounds based on their permeability. It has been shown, for example, that the apparent permeability values measured in Caco-2 monolayers in the range of 1×10⁻⁷ cm/sec or less typically correlate with poor human absorption (Artursson P, K. J. (1991). Permeability can also be determined using an artificial membrane as a model of a gastrointestinal mucosa. For example, a synthetic membrane can be impregnated with e.g. lecithin and/or dodecane to mimic the net permeability characteristics of a gastrointestinal mucosa. The membrane can be used to separate a compartment containing the phospholipase inhibitor from a compartment where the rate of permeation will be monitored. “Correlation between oral drug absorption in humans and apparent drug.” Biochemical and Biophysical Research Communications 175(3): 880-885.) Also, parallel artificial membrane permeability assays (PAMPA) can be performed. Such in vitro measurements can reasonably indicate actual permeability in vivo. See, for example, Wohnsland et al. J. Med. Chem., 2001, 44:923-930; Schmidt et al., Millipore corp. Application note, 2002, n^{o} AN1725EN00, and n^{o} AN1728EN00, incorporated herein by reference. The permeability coefficient is reported as its decimal logarithm, Log Pe.

In some embodiments, the phospholipase inhibitor permeability coefficient Log Pe is preferably lower than about −4, or lower than about −4.5, or lower than about −5, more preferably lower than about −5.5, and even more preferably lower than about −6 when measured in the permeability experiment described in Wohnsland et al. J. Med. Chem. 2001, 44. 923-930.

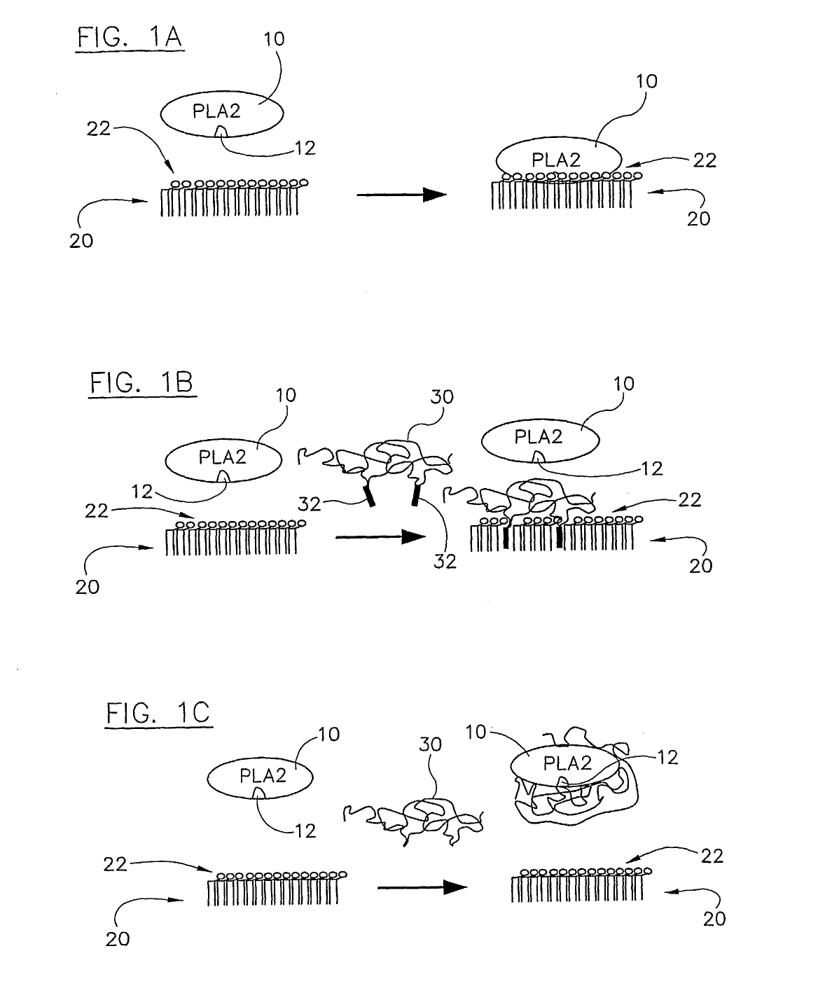

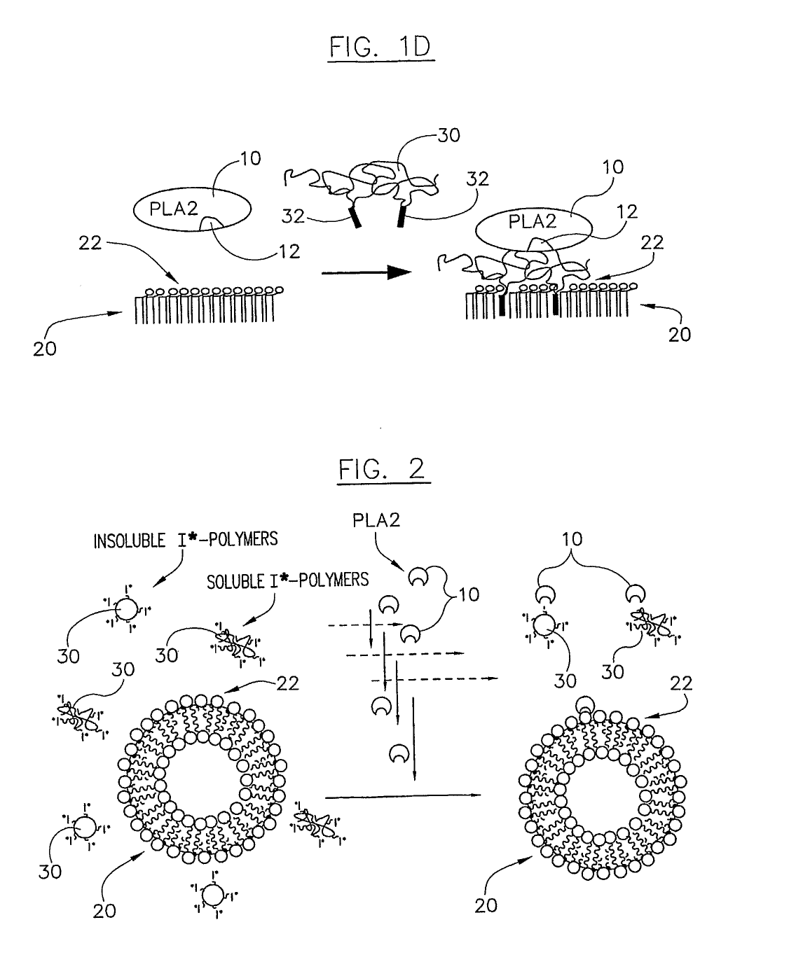

As noted, in one general embodiment, the phospholipase inhibitor can comprise or consist essentially of an oligomer or a polymer. Generally, such polymer inhibitor can be sized to be non-absorbed, and can be adapted to be enzyme-inhibiting, for example based on one or more or a combination of features, such as charge characteristics, relative balance and/or distribution of hydrophilic/hydrophobic character, and molecular structure. The oligomer or polymer in this general embodiment is preferably soluble, and can preferably be a copolymer (including polymers having two monomer-repeat-units, terpolymers and higher-order polymers), including for example random copolymer or block copolymer. The oligomer or polymer can generally include one or more ionic monomer moieties such as one or more anionic monomer moieties. The oligomer or polymer can generally include one or more hydrophobic monomer moieties. The oligomer or polymer inhibitor can interact with the phospholipase, for example with a specific site thereon, preferably with the catalytic site bearing face (e.g., the i-face) of a phospholipase such as phospholipid-A₂. As described below in connection with FIGS. 1A through 1D, the oligomer or polymer can hinder access of a phospholipase to a phospholipids, for example by interacting with the phospholipase, or by interacting with the phospholipid substrate, or by interacting with both the phospholipase and the phospholipid. As described below in connection with FIG. 1C, the inhibitor can be effective for scavenging phospholipase, for example, within a fluid such as an aqueous phase of the gastrointestinal tract.

Specific polymers and specific monomers for such oligomer or polymer inhibitor can be those included in the following discussion, in connection with the general embodiment in which an oligomer or polymer moiety is covalently linked to a phospholipase inhibiting moiety.

In a second general embodiment, a phospholipase inhibitor can comprises a phospholipase inhibiting moiety linked, coupled or otherwise attached to a non-absorbed oligomer or polymer moiety, where such oligomer or polymer moiety can be a hydrophobic moiety, hydrophilic moiety, and/or charged moiety. In some preferred embodiments, the phospholipase inhibiting moiety is coupled to a polymer moiety.

In one more specific approach within this general embodiment, the polymer moiety may be of relatively high molecular weight, for example ranging from about 1000 Da to about 500,000 Da, preferably in the range of about 5000 to about 200,000 Da, and more preferably sufficiently high to hinder or preclude (net) absorption through a gastrointestinal mucosa. Large polymer moieties may be advantageous, for example, in scavenging approaches involving relatively large, soluble or insoluble (e.g., cross-linked) polymers having multiple inhibiting moieties (e.g., as discussed below in connection with FIG. 2).

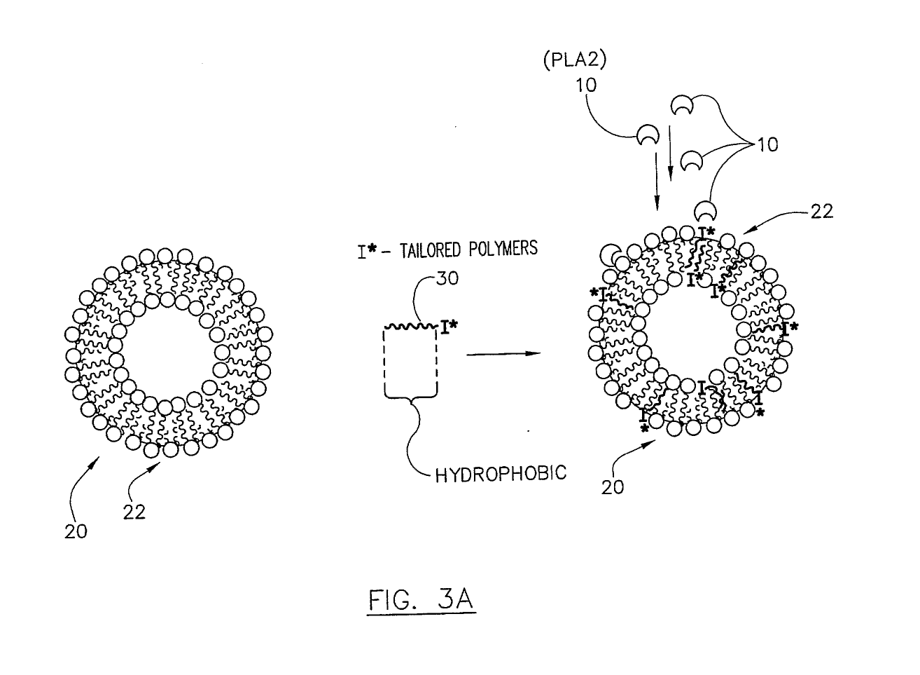

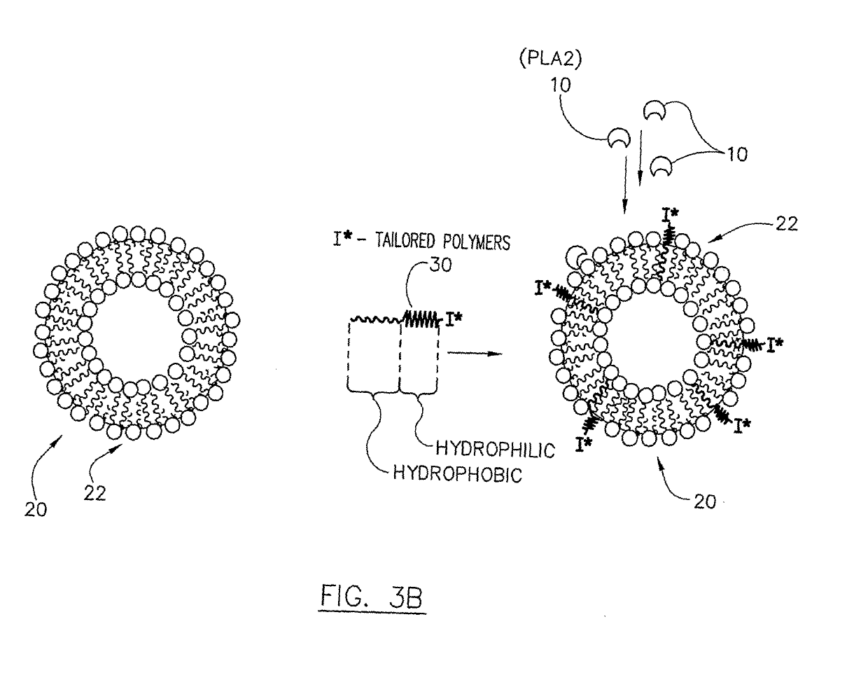

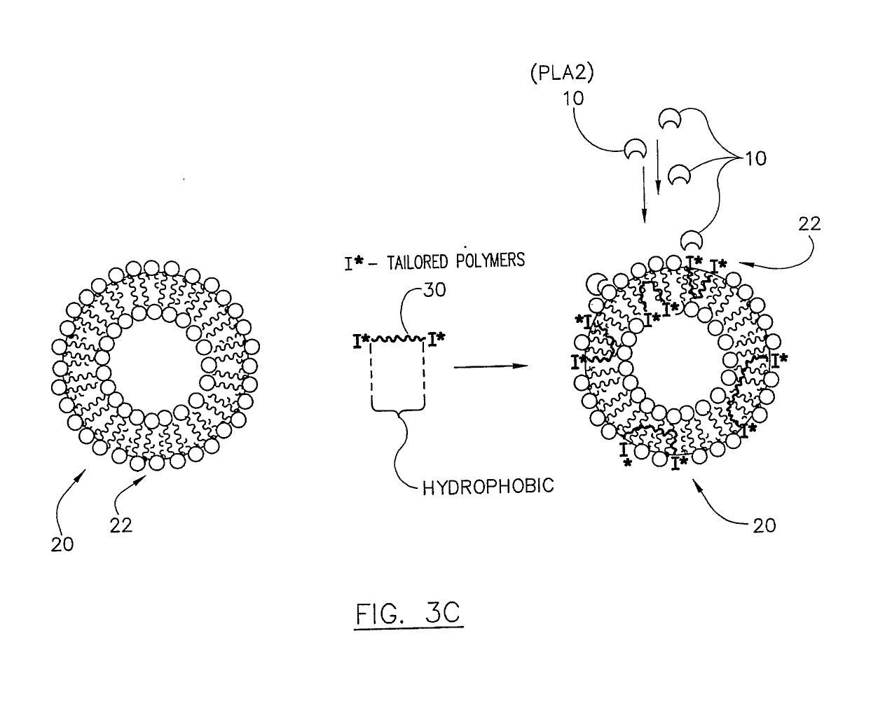

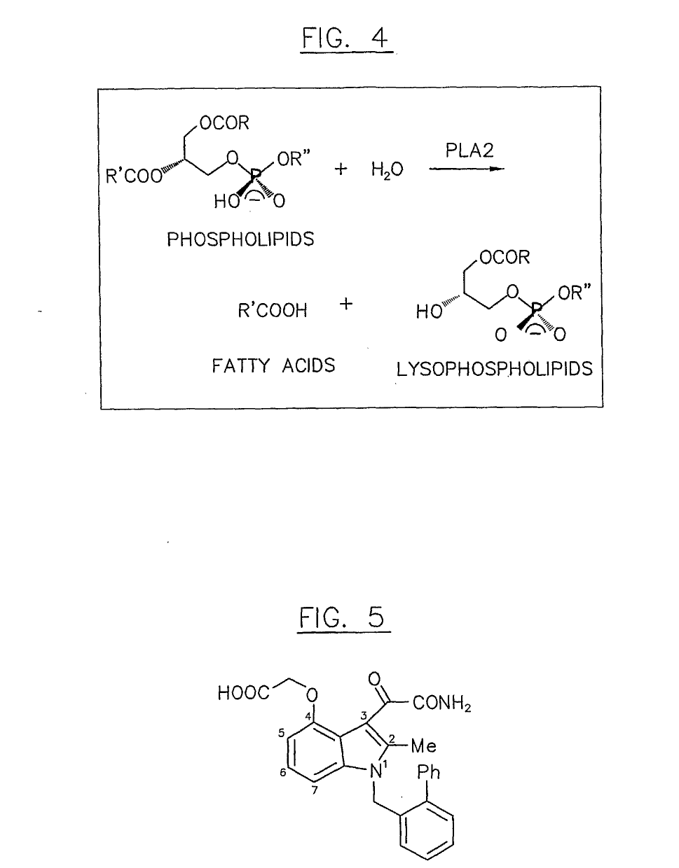

In an alternative more specific approach within this general embodiment, the oligomer or polymer moiety may be of low molecular weight, for example not more than about 5000 Da, and preferably not more than about 3000 Da and in some cases not more than about 1000 Da. Preferably within this approach, the oligomer or polymer moiety can consist essentially of or can comprise a block of hydrophobic polymer, allowing the inhibitor to associate with a water-lipid interface (e.g., of a lipid aggregate as described below in connection with FIGS. 3A through 3C).

In any case, and particularly for each of the immediately aforementioned more specific approaches for this general embodiment, a phospholipase inhibiting moiety may be linked to at least one repeat unit of a polymer moiety. Hence, the phospholipase inhibitor can comprise a repeat unit, an oligomer or a polymer according to the following formula (A):

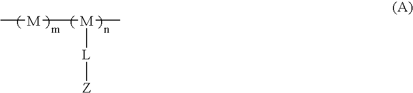

where n and m are each integers (at least one of which is a non-zero integer), M represents a monomer moiety, L is an optional linking moiety, (e.g., a chemical linker), and Z is a phospholipase inhibiting moiety, preferably a PL A₂ inhibiting moiety, and most preferably a PL A₂ 1B inhibiting moiety. Generally, n can be less than 1000; in some embodiments, n can be less than about 500. Preferably, n is at least 2 and less than about 500.

Generally, M represents one or more monomer moiety. Accordingly, each M can independently include one or more of a first monomer moiety, M₁, a second monomer moiety, M₂, a third monomer moiety, M₃, a fourth monomer moiety, M₄, a fifth monomer moiety, M₅, a sixth monomer moiety, M₆, etc., in each case with M₁ through M₆ being different from each other.

In one approach, each M can be one monomer moiety (the same type repeat unit), such that the phospholipase inhibitor can comprises a repeat unit, an oligomer or a polymer having the formula (A-1) 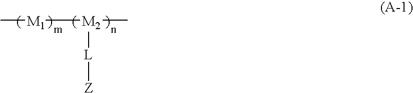
wherein m is a non-zero integer, n is a non-zero integer, M₁ is a first monomer moiety, M₂ is a second monomer moiety, the second monomer moiety being the same as or different than the first monomer moiety, L is an optional linking moiety and Z is a phospholipase inhibiting moiety. In this case, each of M₁ and M₂ can be the same, whereby the phospholipase inhibitor comprises a homopolymer repeat unit, oligomer or polymer moiety. Alternatively, M₁ and M₂ can be different, whereby the phospholipase inhibitor comprises a copolymer repeat unit, oligomer or polymer moiety. The copolymer repeat unit, oligomer or polymer moiety can be a random copolymer or a block copolymer repeat unit, oligomer or polymer moiety. Generally, in some embodiments, n can be less than about 500. Preferably, n is at least 2 and less than about 500.

In a preferred embodiment, the phospholipase inhibitor can comprises an oligomer or polymer moiety having a first repeat unit and a second repeat unit, the first repeat unit having a formula (A-1), above, wherein n is one and m is one or more, whereby the oligomer or polymer moiety of the phospholipase inhibitor is a random copolymer comprising the first and second repeat units. Preferably, m ranges from four to fifty and n is two. More preferably, m is at least four and n is one. The second repeat unit can be of any suitable monomer type.

In some preferred embodiments, for example, where the oligomer or polymer moiety is of a relatively low molecular weight, the oligomer or polymer moiety can be a tailored oligomer or polymer moiety adapted to associate with a water-lipid interface (e.g., of a lipid aggregate as described below in connection with FIGS. 3A through 3C). In such embodiments, the oligomer or polymer moiety can consist essentially of or can comprise a region or block having a relatively hydrophobic character, allowing for integral association with the lipid aggregate (e.g., micelle or vesicle).

For example, in this regard, the phospholipase inhibitor can comprises a compound of the formula (B) 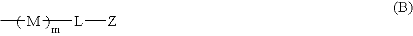
wherein m is a non-zero integer, M is a monomer moiety, L is an optional linking moiety and Z is a phospholipase inhibiting moiety. Such oligomer or polymer moieties having a single covalently-linked inhibiting moiety can be referred to herein as a “singlet” inhibitor and can be effective, for example, as illustrated and discussed below in connection with FIGS. 3A and 3B.

As another example, the phospholipase inhibitor can comprise an oligomer or polymer moiety covalently linked to a phospholipase inhibiting moiety, the phospholipase inhibitor comprising a compound having the formula (C) 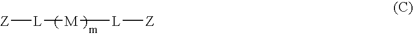
wherein m is a non-zero integer, M is a monomer moiety, L are each independently selected optional linking moieties and Z are each, independently selected phospholipase inhibiting moieties. As a further example, the phospholipase inhibitor can comprise an oligomer or polymer moiety covalently linked to a phospholipase inhibiting moiety, the phospholipase inhibitor comprising a compound having the formula (C-1) 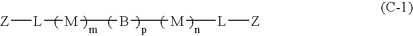
wherein m is a non-zero integer, n is a non-zero integer, p is a non-zero integer, M are each independently selected monomer moieties, B is a bridging moiety, L are each independently selected optional linking moieties, and Z are each independently selected phospholipase inhibiting moieties. In each of these two cases, such oligomer or polymer moieties having two covalently-linked inhibiting moieties can be referred to herein as a “dimer” inhibitor and can be effective, for example, as illustrated and discussed below in connection with Formula C.

In these immediately preceding singlet and dimer embodiments, M represents one or more monomer moiety, and each M can independently include one or more of a first monomer moiety, M₁, a second monomer moiety, M₂, a third monomer moiety, M₃, a fourth monomer moiety, M₄, a fifth monomer moiety, M₅, a sixth monomer moiety, M₆, etc., in each case with M₁ through M₆ being different from each other. In some cases, M can generally comprise at least a first monomer moiety, M₁, and optionally further comprises in combination therewith a second monomer moiety, M₂, different from the first monomer moiety. M can consist essentially of a first monomer, M₁, whereby the phospholipase inhibitor comprises a homopolymer oligomer or polymer moiety or moieties. Alternatively, M can comprise a first monomer, M₁, and a second monomer, M₂ different from the first monomer, whereby the phospholipase inhibitor comprises a copolymer oligomer or polymer moiety or moieties. The copolymer oligomer or polymer moiety can be random copolymer or a block copolymer moiety or moieties. M can generally comprise a hydrophobic monomer moiety, and can also include generally an anionic monomer moiety. In one specific example, M can comprise a first block consisting essentially of a hydrophobic first monomer, M₁, and a second block consisting essentially of a hydrophilic second monomer, M₂, with the second block being proximal to the phospholipase inhibiting moiety or moieties. In these embodiments, m can range from four to about fifty.

Hence, in one embodiment, the phospholipase inhibitor can comprise a compound of the formula (C-2) 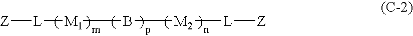
wherein m is a non-zero integer, n is a non-zero integer, p is a non-zero integer, M₁ is a first monomer moiety, M₂ is a second monomer moiety, the second monomer moiety being the same as or different than the first monomer moiety, B is a bridging moiety, L are each independently selected optional linking moieties, and Z are each independently selected phospholipase inhibiting moieties. In this embodiment, m and n can each be independently selected integers ranging from four to about 500, preferably ranging from four to about 100, and most preferably ranging from four to fifty.

The linking moiety L, in each of the described embodiments, can be a chemical linker, such as a bond or a other moiety, for example, comprising about 1 to about 10 atoms that can be hydrophilic and/or hydrophobic. The linking moiety links, couples, or otherwise attaches the phospholipase inhibiting moiety Z to the polymer moiety, for example to a backbone of the polymer moiety. In one embodiment, the linking moiety can be a polymer moiety grafted onto a polymer backbone, for example, using living free radical polymerization approaches known in the art.

Generally, with respect to the polymer moiety, a number of polymers can be used including, for example, synthetic and/or naturally occurring aliphatic, alicyclic, and/or aromatic polymers. In preferred embodiments, the polymer moiety is stable under physiological conditions of the gastrointestinal (GI) tract. By “stable” it is meant that the polymer moiety does not degrade or does not degrade significantly or essentially does not degrade under the physiological conditions of the GI tract. For instance, at least about 90%, preferably at least about 95%, and more preferably at least about 98%, and even more preferably at least about 99% of the polymer moiety remains un-degraded or intact after at least about 5 hours, at least about 10 hours, at least about 24 hours, or at least about 48 hours of residence in a gastrointestinal tract (in each case based on a statistically relevant data set). Stability in a gastrointestinal tract can be evaluated using gastrointestinal mimics, e.g., gastric mimics or intestinal mimics of the small intestine, which approximately model the physiological conditions at one or more locations within a GI tract.

The polymer moiety may be soluble or insoluble, existing for example as dispersed micelles or particles, such as colloidal particles or (insoluble) macroscopic beads. In some embodiments, the polymer moiety presents as insoluble porous particles. In preferred embodiments, the polymer moiety is soluble or exists as colloidal dispersions under the physiological conditions of the gastrointestinal tract, for example, at a location within the GI tract where the phospholipase inhibiting moiety acts, e.g., within the gastrointestinal lumen of the small intestine.

Polymer moieties can be hydrophobic, hydrophilic, amphiphilic, uncharged or non-ionic, negatively or positively charged, or a combination thereof, and can be organic or inorganic. Inorganic polymers, also referred to as inorganic carriers in some cases, include silica (e.g., multi-layered silica), diatomaceous earth, zeolite, calcium carbonate, talc, and the like.

The polymer architecture of the polymer moiety can be linear, grafted, comb, block, star and/or dendritic, preferably selected to produce desired solubility and/or stability characteristics as described above. The architecture may involve a macromolecular scaffold, and in some embodiments the scaffold may form particles that may be porous or non-porous. The particles may be of any shape, including spherical, elliptical, globular, or irregularly-shaped particles. Preferably the particles are composed of a crosslinked organic polymer derived from, e.g., styrenic, acrylic, methacrylic, allylic, or vinylic monomers, or produced by polycondensation such as polyester, polyamide, melamin and phenol formol condensates, or derived from semi-synthetic cellulose and cellulose-like materials, such as cross-linked dextran or agarose (e.g., Sepharose (Amersham)).

In preferred particle embodiments comprising a phospholipase inhibiting moiety linked, coupled or otherwise attached to a polymer moiety, the particles provide enough available surface area to allow binding of the phospholipase inhibiting moiety to phospholipase. For example, in order to help reduce the dose required to produce a therapeutic and/or a prophylactic benefit, the particles should exhibit specific surface area in the range of about 2 m²/gr to about 500 m²/gr, preferably about 20 m²/gr to about 200 m²/gr, more preferably about 40 m²/gr to about 100 m²/gr.

Phospholipase inhibiting moieties are preferably linked, coupled or otherwise attached to the polymer moiety on the surface of such particles and preferably at a density of about 0.05 mmol/g to about 4 mmol/g of the polymer moiety, more preferably about 0.1 mmol/g to about 2 mmol/g of the polymer moiety. The density of phospholipase inhibiting moieties can be determined, for example, taking into account the amount of overall PLA2 enzyme typically encountered in the human GI during or shortly after ingestion of a meal. PLA2 enzyme loading is reported to range from about 150-400 mg/L during the digestion phase with a total duodenal/jejunal volume ranging from about 1 to 2 liters. Based on a mole ratio of enzyme: inhibitor ranging from about 1:10 to about 1:100 (in a treatment protocol involving administering of PLA2 inhibitor during or shortly after meals), the mole content of inhibitor relative to moles polymer, expressed as immobilized inhibiting moieties within a polymer particle, can range from about 0.01 to about 100 mEq, and preferably from about 0.1 to about 50 mEq. The overall capacity of inhibiting-moiety-containing particles can be between about 0.05 to about 5 mEq/g, preferably from about 0.1 to about 2.5 mEq/g, and the oral administration of such inhibiting-moiety-containing particles can be between about 0.1 g and 10 g, and preferably between about 0.5 g to 5 g.

In the case where the polymer moiety forms porous particles, beads, or matrices, the pore dimension can be large enough to accommodate phospholipase, e.g., PL A₂, within the pores. In some embodiments, for example, porosity may be selected such that the minimum pore size is at least about 2 nm, preferably at least about 5 nm, and more preferably at least about 20 nm. Such materials can be produced by direct or inverse suspension polymerization using process additives such as diluent, porogen, and/or suspension aids, which can control size and porosity.

Polymer moieties useful in constructing non-absorbed inhibitors of the present invention can also be produced by free radical polymerization, condensation, addition polymerization, ring-opening polymerization, and/or can be derived from naturally occurring polymers, such as saccharide polymers. Further, in some embodiments, any of these polymer moieties may be functionalized.

Examples of polysaccharides useful in the present invention include materials from vegetal or animal origin, including cellulose materials, hemicellulose, alkyl cellulose, hydroxyalkyl cellulose, carboxymethylcellulose, sulfoethylcellulose, starch, xylan, amylopectine, chondroitin, hyarulonate, heparin, guar, xanthan, mannan, galactomannan, chitin, and/or chitosan. As noted above, more preferred are polymer moieties that do not degrade or that do not degrade significantly or essentially do not degrade under the physiological conditions of the GI tract, such as carboxymethylcellulose, chitosan, and sulfoethylcellulose.

When free radical polymerization is used, the polymer moiety can be prepared from various classes of monomers including, for example, acrylic, methacrylic, styrenic, vinylique dienic, whose typical examples are given thereafter: styrene, substituted styrene, alkyl acrylate, substituted alkyl acrylate, alkyl methacrylate, substituted alkyl methacrylate, acrylonitrile, methacrylonitrile, acrylamide, methacrylamide, N-alkylacrylamide, N-alkylmethacrylamide, N,N-dialkylacrylamide, N,N-dialkylmethacrylamide, isoprene, butadiene, ethylene, vinyl acetate, and combinations thereof. Functionalized versions of these monomers may also be used and any of these monomers may be used with other monomers as comonomers. For example, specific monomers or comonomers that may be used in this invention include methyl methacrylate, ethyl methacrylate, propyl methacrylate (all isomers), butyl methacrylate (all isomers), 2-ethylhexyl methacrylate, isobornyl methacrylate, methacrylic acid, benzyl methacrylate, phenyl methacrylate, methacrylonitrile, α-methylstyrene, methyl acrylate, ethyl acrylate, propyl acrylate (all isomers), butyl acrylate (all isomers), 2-ethylhexyl acrylate, isobornyl acrylate, acrylic acid, benzyl acrylate, phenyl acrylate, acrylonitrile, styrene, glycidyl methacrylate, 2-hydroxyethyl methacrylate, hydroxypropyl methacrylate (all isomers), hydroxybutyl methacrylate (all isomers), N,N-dimethylaminoethyl methacrylate, N,N-diethylaminoethyl methacrylate, triethyleneglycol methacrylate, itaconic anhydride, itaconic acid, glycidyl acrylate, 2-hydroxyethyl acrylate, hydroxypropyl acrylate (all isomers), hydroxybutyl acrylate (all isomers), N,N-dimethylaminoethyl acrylate, N,N-diethylaminoethyl acrylate, triethyleneglycol acrylate, methacrylamide, N-methylacrylamide, N,N-dimethylacrylamide, N-tert-butylmethacrylamide, N-n-butylmethacrylamide, N-methylolmethacrylamide, N-ethylolmethacrylamide, N-tert-butylacrylamide, N-n-butylacrylamide, N-methylolacrylamide, N-ethylolacrylamide, 4-acryloylmorpholine, vinyl benzoic acid (all isomers), diethylaminostyrene (all isomers), a-methylvinyl benzoic acid (all isomers), diethylamino α-methylstyrene (all isomers), p-vinylbenzene sulfonic acid, p-vinylbenzene sulfonic sodium salt, alkoxy and alkyl silane functional monomers, maleic anhydride, N-phenylmaleimide, N-butylmaleimide, butadiene, isoprene, chloroprene, ethylene, vinyl acetate, vinylformamide, allylamine, vinylpyridines (all isomers), fluorinated acrylate, methacrylates, and combinations thereof. Main chain heteroatom polymer moieties can also be used, including polyethyleneimine and polyethers such as polyethylene oxide and polypropylene oxide, as well as copolymers thereof.

Generally, the number of phospholipase inhibiting moieties Z appended to the polymer moiety can vary from about 1 to about 2000, most preferably from about 1 to about 500. These phospholipase inhibiting moieties can be arranged regularly or randomly along a backbone of the polymer moiety or can be localized in one particular region of the polymer moiety. For instance, (M) and (M-L-Z) repeat units can be arranged regularly, e.g., in sequences, or randomly along a backbone of the polymer moiety. If block copolymers are used, the phospholipase inhibiting moieties can be present on one block while not on another block.

The phospholipase inhibiting moiety Z may be any art-known phospholipase inhibitor, and/or any phospholipase inhibiting moiety described herein. Preferably, the phospholipase inhibitor comprises a phospholipase inhibiting moiety that is active under the physiological conditions of the GI tract, e.g. within the pH range prevailing within the gastrointestinal lumen, i.e., from about 5 to about 8, and preferably under physiological conditions prevailing at a location within the GI tract where the phospholipase inhibiting moiety acts, e.g., within the gastrointestinal lumen of the small intestine.

In some embodiments, non-absorbed PL A₂ inhibitors of the invention comprise an art-known PL A₂ inhibiting moiety. Art-know PL A₂ inhibiting moieties include, for example, small molecule inhibitors of phospholipase A2, such as FPL 67047XX and/or MJ99. Other phospholipase inhibitors useful in the practice of the methods of this invention include arachidonic acid analogues (e.g., arachidonyl trifluoromethyl ketone, methylarachidonyl fluorophosphonate, and palmitoyl trifluoromethyl ketone), benzensulfonamide derivatives, bromoenol lactone, p-bromophenyl bromide, bromophenacyl bromide, trifluoromethylketone, sialoglycolipids, proteoglycans, and the like, as well as phospholipase A2 inhibitors disclosed in WO 03/101487, incorporated herein by reference.

Art-know PL A₂ inhibiting moieties useful in this invention also include, for example, phospholipid analogs and structures developed to target secreted PL A₂, for example, for indications such as obstructive respiratory disease (including asthma), colitis, Crohn's disease, central nervous system insult, ischemic stroke, multiple sclerosis, contact dermatitis, psoriasis, cardiovascular disease (including arteriosclerosis), autoimmune disease, and other inflammatory states.

Phospholipid analogs useful as phospholipase inhibiting moieties of some phospholipase inhibitors of this invention include structural analogs of a phospholipid substrate and/or its transition state, which can comprise one or more classes of compounds known in the art to resemble phospholipid substrates and/or their transition states, preferably resembling their polar head groups rather than their long chain hydrophobic groups. Such analog inhibitors can include, for example, compounds disclosed in Gelb M., Jain M., Berg O., Progress in Surgery, Principles of inhibition of phospholipase A2 and other interfacial enzymes, 1997, 24:123-129, for example, see Table 1 therein, incorporated herein by reference. Examples of PL A₂ inhibiting moieties in some preferred embodiments are provided below:

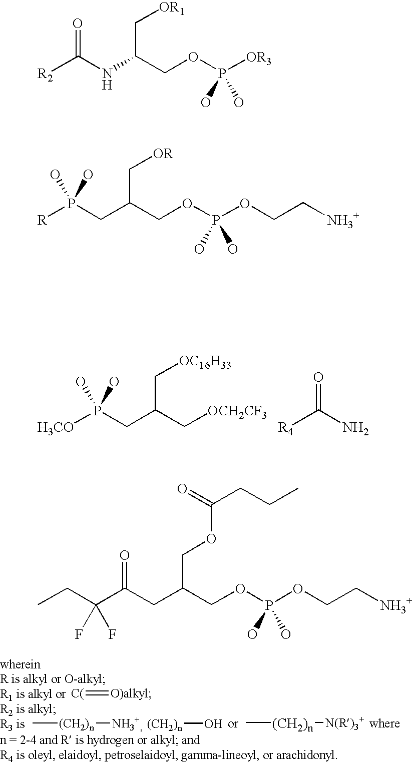

Phospholipid analogs useful as phospholipase inhibiting moieties of some phospholipase inhibitors of this invention also include phosphonate-containing compounds, such as those disclosed in Lin et al, J. Am. Chem. Soc., 115 (10) 1993, preferably the compounds represented by the structures provided below:

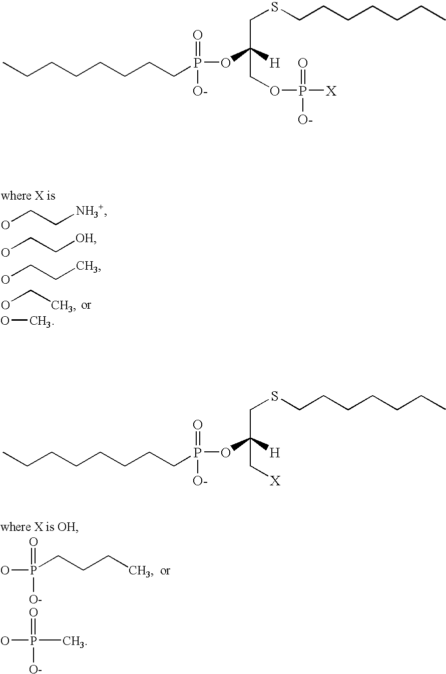

Transition state analogs useful as phospholipase inhibiting moieties of some phospholipid inhibitors of the present invention include one or more compounds taught in Jain, M et al., Biochemistry, 1991, 30:10256-10268, for example, see Tables IV, V and VI therein, incorporated herein by reference. In some preferred embodiments, inhibitors of the present invention comprise a moiety derived from modified glycerol backbone (see, for example, table VI of Jain, 1991), which have proven to be potent inhibitors of pancreatic PL A₂, including, for example, the structures illustrated below:

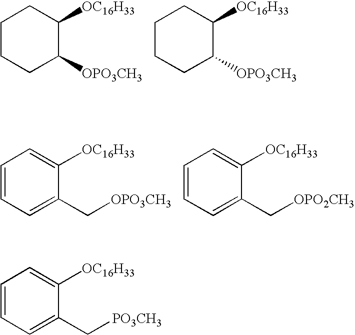

In some preferred embodiments, described below, the phospholipase-A2 inhibitor (or inhibiting moiety) can comprise indole compounds or indole-related compounds.

In general, therefore, preferred embodiments of the various aspects of the invention, the phospholipase inhibitor (or inhibiting moiety) can comprise a substituted organic compound (or moiety derived from a substituted organic compound) having a fused five-member ring and six-member ring (or as a pharmaceutically-acceptable salt thereof). Preferably, the inhibitor (or inhibiting moiety) also comprises substituent groups effective for imparting phospholipase-A2 inhibiting functionality to the inhibitor (or inhibiting moiety), and preferably phospholipase-A2 IB inhibiting functionality. Preferably the phospholipase inhibitor (inhibiting moiety) is a fused five-member ring and six-member ring having one or more heteroatoms (e.g., nitrogen, oxygen, sulfur) substituted within the ring structure of the five-member ring, within the ring structure of the six-member ring, or within the ring structure of each of the five-member and six-member rings (or as a pharmaceutically-acceptable salt thereof). Again preferably, the inhibitor (or inhibiting moiety) can comprise substituent groups effective for imparting phospholipase inhibiting functionality to the moiety.

As demonstrated in Example 10 (including related Examples 10A through 10C), substituted organic compounds (or moieties derived therefrom) having such fused five-member ring and six-member ring are effective phospholipase-2A IB inhibitors, with phenotypic effects approaching and/or comparable to the effect of genetically deficient PLA2 (−/−) mice. Moreover, such compound (or moieties derived therefrom) are effective in treating conditions such as weight-related conditions, insulin-related conditions, and cholesterol-related conditions, including in particular conditions such as obesity, diabetes mellitus, insulin resistance, glucose intolerance, hypercholesterolemia and hypertriglyceridemia.

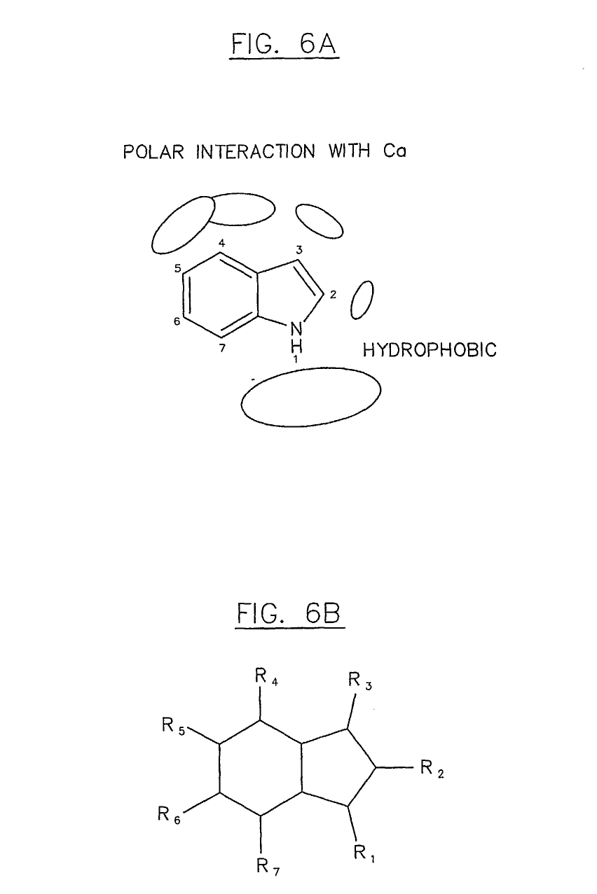

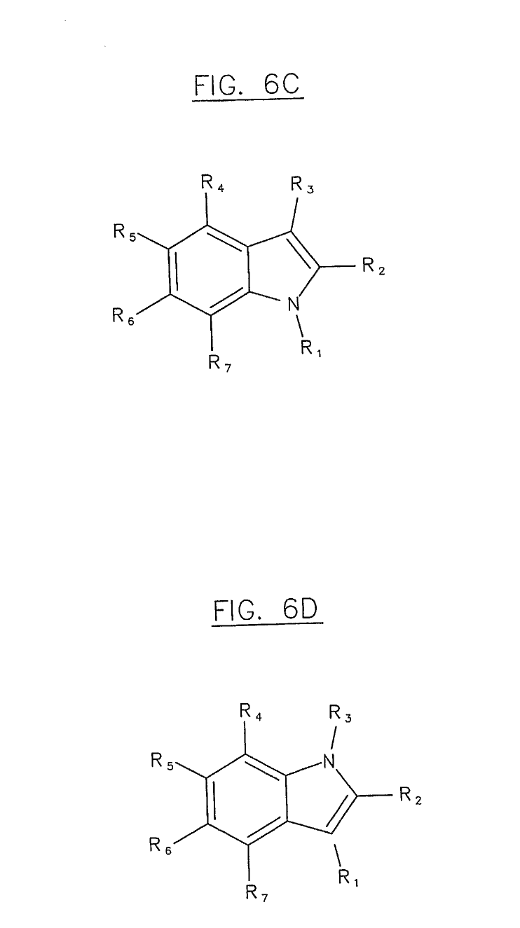

Although a particular compound was evaluated in-vivo in the study described in Example 10, namely the compound 2-(3-(2-amino-2-oxoacetyl)-1-(biphenyl-2-ylmethyl)-2-methyl-1H-indol-4-yloxy)acetic acid, shown in FIG. 5, the results of this study support a more broadly-defined invention, because the inhibitive effect can be realized and understood through structure-activity-relationships as described in detail hereinafter. Briefly, without being bound by theory not specifically recited in the claims, compounds comprising the fused five-membered and six-membered rings have a structure that advantageously provides an appropriate bond-length and bond-angles for positioning substituent groups—for example at positions 3 and 4 of an indole-compound as represented in FIG. 6A, and at the —R₃ and —R₄ positions of the indole-related compounds comprising fused five-membered and six-membered rings as represented in FIG. 6B. Mirror-image analogues of such indole compounds and of such indole-related compounds also can be used in connection with this invention, as described below.

In particularly preferred embodiments, the phospholipase-A2 inhibiting moiety can comprise a fused five-membered ring and six-membered ring as a compound (or as a pharmaceutically-acceptable salt thereof), represented by the following formula (I): 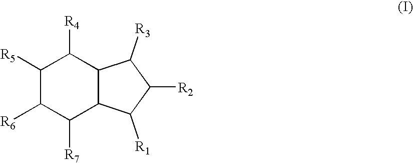
wherein the core structure can be saturated (as shown above) or unsaturated (not shown), and wherein R₁ through R₇ are independently selected from the group consisting of: hydrogen, oxygen, sulfur, phosphorus, amine, halide, hydroxyl (—OH), thiol (—SH), carbonyl, acidic, alkyl, alkenyl, carbocyclic, heterocyclic, acylamino, oximyl, hydrazyl, substituted substitution group, and combinations thereof; and additionally or alternatively, wherein R₁ through R₇ can optionally comprise, independently selected additional rings between two adjacent substitutents, with such additional rings being independently selected 5-, 6-, and/or 7-member rings which are carbocyclic rings, heterocyclic rings, and combinations thereof.

As used generally herein, including as used in connection with R₁ through R₇ in the indole-related compound shown above:

an amine group can include primary, secondary and tertiary amines;

a halide group can include fluoro, chloro, bromo, or iodo;

a carbonyl group can be a carbonyl moiety having a further substitution (defined below) as represented by the formula

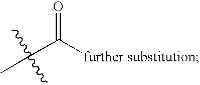

an acidic group can be an organic group as a proton donor and capable of hydrogen bonding, non-limiting examples of which include carboxylic acid, sulfate, sulfonate, phosphonates, substituted phosphonates, phosphates, substituted phosphates, 5-tetrazolyl,

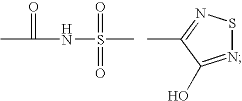

an alkyl group by itself or as part of another substituent can be a substituted or unsubstituted straight or branched chain hydrocarbon such as methyl, ethyl, n-propyl, isopropyl, n-butyl, tertiary butyl, sec-butyl, n-pentyl, n-hexyl, decyl, dodecyl, or octadecyl;

an alkenyl group by itself or in combination with other group can be a substituted or unsubstituted straight chain or branched hydrocarbon containing unsaturated bonds such as vinyl, propenyl, crotonyl, isopentenyl, and various butenyl isomers;

a carbocyclic group can be a substituted or unsubstituted, saturated or unsaturated, 5- to 14-membered organic nucleus whose ring forming atoms are solely carbon atoms, including cycloalkyl, cycloalkenyl, phenyl, spiro[5.5]undecanyl, napthyl, norbornanyl, bicycloheptadienyl, toluoyl, xylenyl, indenyl, stilbenzyl, terphenylyl, diphenylethylenyl, phenyl-cyclohexenyl, acenaphthylenyl, and anthracenyl, biphenyl, and bibenzylyl;

a heterocyclic group can be monocyclic or polycyclic, saturated or unsaturated, substituted or unsubstituted heterocyclic nuclei having 5 to 14 ring atoms and containing from 1 to 3 hetero atoms selected from the group consisting of nitrogen, oxygen or sulfur, including pyrrolyl, pyrrolodinyl, piperidinyl, furanyl, thiophenyl, pyrazolyl, imidazolyl, phenylimidazolyl, triazolyl, isoxazolyl, oxazolyl, thiazolyl, thiadiazolyl, indolyl, carbazolyl, norharmanyl, azaindolyl, benzofuranyl, dibenzofuranyl, dibenzothiophenyl, indazolyl, imidazo pyridinyl, benzotriazolyl, anthranilyl, 1,2-benzisoxazolyl, benzoxazolyl, benzothiazolyl, purinyl, pyridinyl, dipyridylyl, phenylpyridinyl, benzylpyridinyl, pyrimidinyl, phenylpyrimidinyl, pyrazinyl, 1,3,5-triazinyl, quinolinyl, phthalazinyl, quinazolinyl, morpholino, thiomorpholino, homopiperazinyl, tetrahydrofuranyl, tetrahydropyranyl, oxacanyl, 1,3-dioxolanyl, 1,3-dioxanyl, 1,4-dioxanyl, tetrahydrothiophenyl, pentamethylenesulfadyl, 1,3-dithianyl, 1,4-dithianyl, 1,4-thioxanyl, azetidinyl, hexamethyleneiminium, heptamethyleneiminium, piperazinyl and quinoxalinyl;

an acylamino group can be an acylamino moiety having two further substitutions (defined below) as represented by the formula:

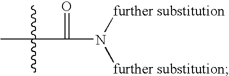

an oximyl group can be an oximyl moiety having two further substitutions (defined below) as represented by the formula:

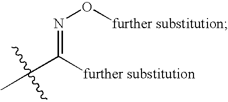

a hydrazyl group can be a hydrazyl moiety having three further substitutions (defined below) as represented by the formula:

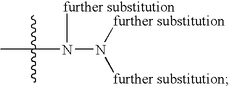

a substituted substitution group combines one or more of the listed substituent groups, preferably through moieties that include for example

an -oxygene-alkyl-acidic moiety such as

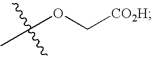

a -carbonyl-acyl amino-hydrogen moiety such as

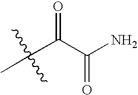

an -alkyl-carbocyclic-alkenyl moiety such as

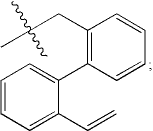

a -carbonyl-alkyl-thiol moiety such as

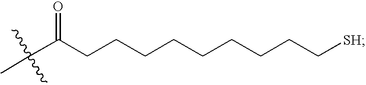

an -amine-carbonyl-amine moiety such as

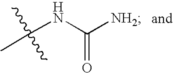

a further substitution group can mean a group selected from hydrogen, oxygen, sulfur, phosphorus, amine, halide, hydroxyl (—OH), thiol (—SH), carbonyl, acidic, alkyl, alkenyl, carbocyclic, heterocyclic, acylamino, oximyl, hydrazyl, substituted substitution group, and combinations thereof.

Particularly preferred substituent groups R₁ through R₇ for such indole-related compounds are described below in connection with preferred indole-compounds.

In preferred embodiments, the phospholipase-A2 inhibiting moiety can comprise an indole compound (e.g., an indole-containing compound or compound containing an indole moiety), such as a substituted indole moiety. For example, in such embodiment, the indole-containing compound can be a compound represented by the formulas II, III (considered left to right as shown): 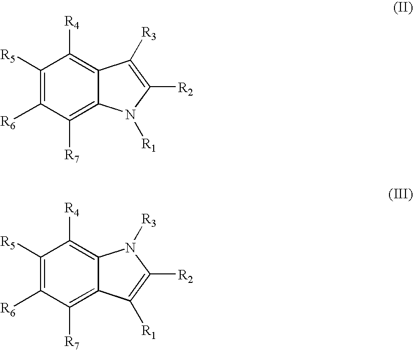
wherein R₁ through R₇ are independently selected from the groups consisting of: hydrogen, oxygen, sulfur, phosphorus, amine, halide, hydroxyl (—OH), thiol (—SH), carbonyl, acidic, alkyl, alkenyl, carbocyclic, heterocyclic, acylamino, oximyl, hydrazyl, substituted substitution group, and combinations thereof; and additionally or alternatively, wherein R₁ through R₇ can optionally, and independently form additional rings between two adjacent substitutents with such additional rings being 5-, 6-, and 7-member ring selected from the group consisting of carbocyclic rings, heterocyclic rings and combinations thereof.

Some indole compounds having additional rings include, for example, those compounds represented as formulas IVa through IVf (considered left to right in top row as IVa, IVb, IVc, and considered left to right bottom row as IVd, IVe and IVf, as shown):

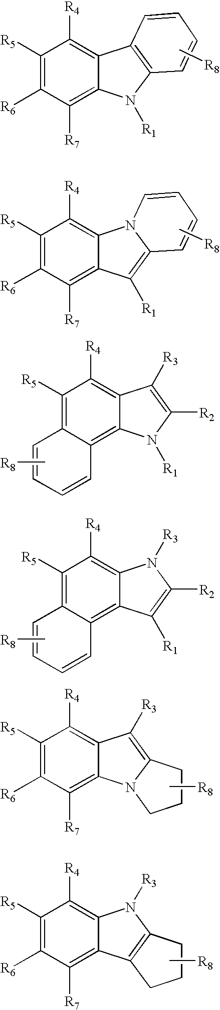

Generally, the various types of substituent groups, including carbonyl, acidic, alkyl, alkenyl, carbocyclic, heterocyclic, acylamino, oximyl, hydrazyl, substituted substitution group, can be as defined above in connection with the indole-related compounds having fused five-membered and six-membered rings.

In each of the embodiments of the invention, including for those compounds that are indole-related compounds having fused five-membered and six-membered rings, and for the indole compounds, preferred substitutent groups can be as described in the following paragraphs.

Preferred R₁ is selected from the following groups: hydrogen, oxygen, sulfur, amine, halide, hydroxyl (—OH), thiol (—SH), carbonyl, acidic, alkyl, alkenyl, carbocyclic, heterocyclic, substituted substitution group and combinations thereof. Particularly preferred R₁ is selected from the following groups: hydrogen, halide, thiol (—SH), carbonyl, acidic, alkyl, alkenyl, carbocyclic, substituted substitution group and combinations thereof. R₁ is especially preferably selected from the group consisting of alkyl, carbocyclic and substituted substitution group. The substituted substitution group for R₁ are especially preferred compounds or moieties such as:

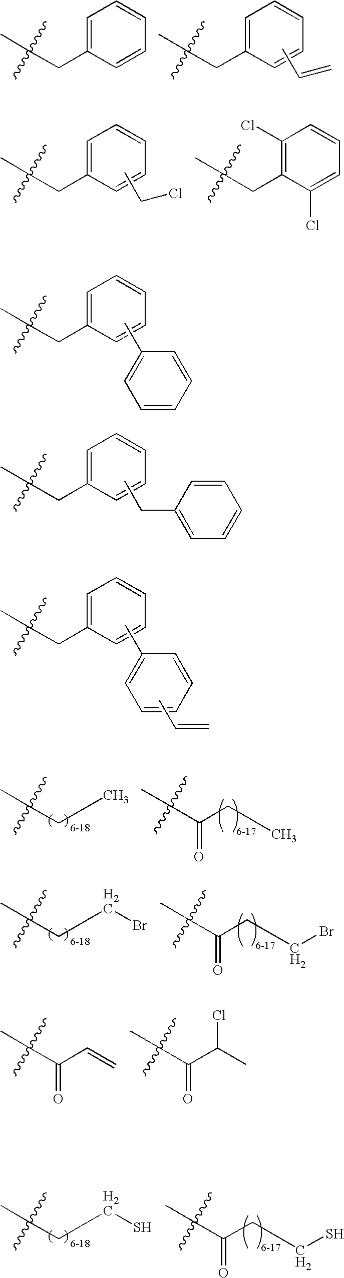

Preferred R₂ is selected from the following groups: hydrogen, oxygen, halide, carbonyl, alkyl, alkenyl, carbocyclic, substituted substitution group, and combinations thereof. Particularly preferred R₂ is selected from the following groups: hydrogen, halide, alkyl, alkenyl, carbocyclic, substituted substitution group, and combinations thereof. R₂ is preferably selected from the group consisting of halide, alkyl and substituted substitution group. The substituted substitution group for R₂ are especially preferred compounds or moieties such as:

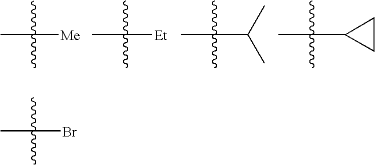

Preferred R₃ is selected from the following groups: hydrogen, oxygen, sulfur, amine, hydroxyl (—OH), thiol (—SH), carbonyl, acidic, alkyl, heterocyclic, acylamino, oximyl, hydrazyl, substituted substitution group and combinations thereof. Particularly preferred R₃ is selected from the following groups: hydrogen, oxygen, amine, hydroxyl (—OH), carbonyl, alkyl, acylamino, oximyl, hydrazyl, substituted substitution group and combinations thereof. R₃ is preferably selected from the group consisting of carbonyl, acylamino, oximyl, hydrazyl, and substituted substitution group. The substituted substitution group for R₃ are especially preferred compounds or moieties such as:

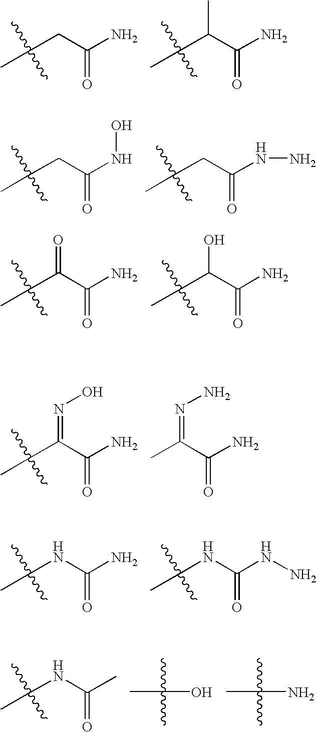

Preferred R₄ and R₅ are independently selected from the following groups: hydrogen, oxygen, sulfur, phosphorus, amine, hydroxyl (—OH), thiol (—SH), carbonyl, acidic, alkyl, alkenyl, heterocyclic, acylamino, oximyl, hydrazyl, substituted substitution group and combinations thereof. Particularly preferred R₄ and R₅ are independently selected from the following groups: hydrogen, oxygen, sulfur, amine, acidic, alkyl, substituted substitution group and combinations thereof. R₄ and R₅ are each preferably independently selected from the group consisting of oxygen, hydroxyl (—OH), acidic, alkyl, and substituted substitution group. The substituted substitution group for R₄ and for R₅ are especially preferred compounds or moieties such as:

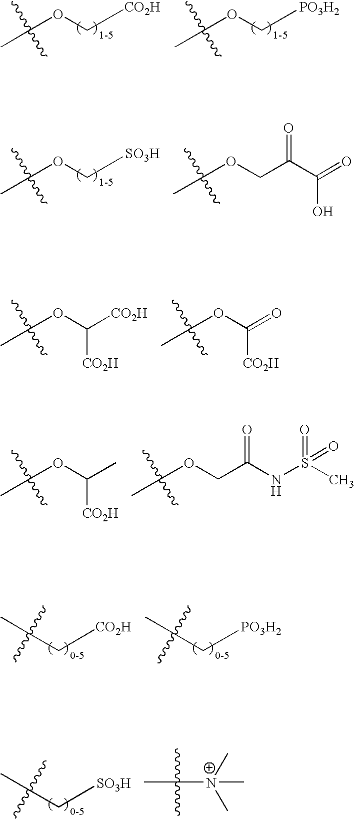

Preferred R₆ is selected from the following groups hydrogen, oxygen, amine, halide, hydroxyl (—OH), acidic, alkyl, carbocyclic, acylamino, substituted substitution group and combinations thereof. Particularly preferred R₆ is selected from the following groups: hydrogen, oxygen, amine, halide, hydroxyl (—OH), acidic, alkyl, acylamino, substituted substitution group and combinations thereof. R₆ is preferably selected from the group consisting of amine, acidic, alkyl, and substituted substitution group. The substituted substitution group for R₆ are especially preferred compounds or moieties such as:

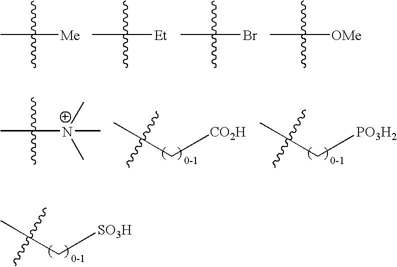

Preferred R₇ is selected from the following groups: hydrogen, oxygen, sulfur, amine, halide, hydroxyl (—OH), thiol (—SH), carbonyl, acidic, alkyl, alkenyl, carbocyclic, heterocyclic, substituted substitution group and combinations thereof. Particularly preferred R₇ is selected from the following groups: hydrogen, halide, thiol (—SH), carbonyl, acidic, alkyl, alkenyl, carbocyclic, substituted substitution group and combinations thereof. R₇ is preferably selected from the groups consisting of carbocyclic and substituted substitution group. The substituted substitution group for R₇ are especially preferred compounds or moieties such as:

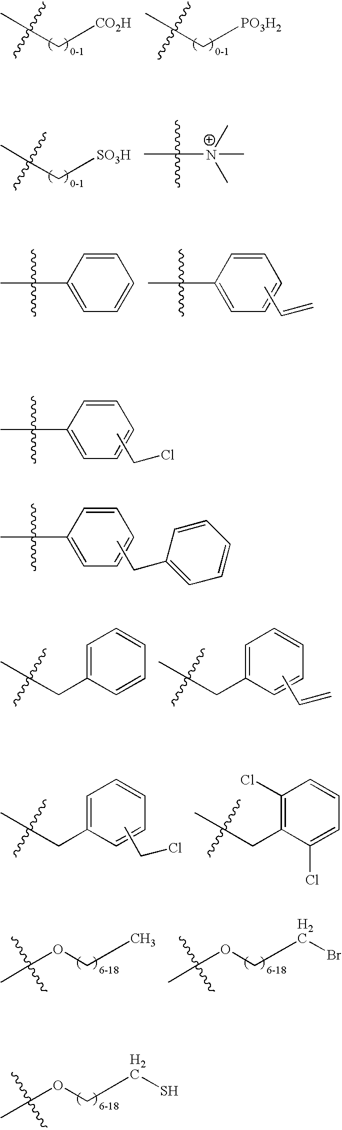

The aforementioned preferred selections for each substituent group R₁ through R₇ can be combined in each variation and permutation. In certain, preferred embodiments, for example, the inhibitor of the invention can comprise substituent groups wherein R₁ through R₇ are as follows: R₁ is preferably selected from the group consisting of alkyl, carbocyclic and substituted substitution group; R₂ is preferably selected from the group consisting of halide, alkyl and substituted substitution group; R₃ is preferably selected from the group consisting of carbonyl, acylamino, oximyl, hydrazyl, and substituted substitution group; R₄ and R₅ are each preferably independently selected from the group consisting of oxygen, hydroxyl (—OH), acidic, alkyl, and substituted substitution group; R₆ is preferably selected from the group consisting of amine, acidic, alkyl, and substituted substitution group; and R₇ is preferably selected from the groups consisting of carbocyclic and substituted substitution group.

Certain indole glyoxamides are particularly useful as PL A₂ inhibiting moieties in some embodiments. Specifically [2-(3-(2-amino-2-oxoacetyl)-1-(biphenyl-2-ylmethyl)-2-methyl-1H-indol-4-yloxy)acetic acid], shown in FIG. 2, alternatively referred to herein as ILY-4001 and/or as methyl indoxam has been found to be an effective phospholipase inhibitor or inhibiting moiety. This indole compound is represented by the structure below, as formula (V):

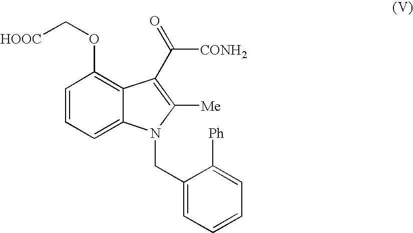

This compound has been shown, based on in-vitro assays, to have phospholipase activity for a number of PLA2 classes, and is a strong inhibitor of mouse and human PLA2IB enzymes in vitro (Singer, Ghomashchi et al. 2002; Smart, Pan et al. 2004). This indole compound was synthesized (See, Example 1A) and was evaluated in-vivo for phospholipase-A2 inhibition in a mice model. (See, Example 10, including Examples 10A through 10C). This indole compound was characterized with respect to inhibition activity, absorption and bioavailability. (See, Example 1B, including Examples 1B-1, 1B-2 and 1B-3).

Bioavailability of this compound can be reduced, and reciprocally, lumen-localization can be improved, according to this second general embodiment of the invention, for example, by covalently linking this indole moiety to a polymer. (See, for example, Example 1D).

Several schemes are described hereinafter to more fully describe the lumen-localization approach for the above compound based on linking the ILY-4001 indole compound as an inhibition moiety to a polymer moiety. Such schemes are included herein to amplify the discussion of the invention; these schemes are not limiting on the invention, and in particular, similar schemes can be employed for other inhibitor moieties.

In a first approach, a functionalized inhibitor can be coupled to a preformed functionalized polymer such as a commercial polymer beads or soluble polymers. For example the linker possesses a halide or an amine to react with amine functionalized or an activated carboxylic acid bead.

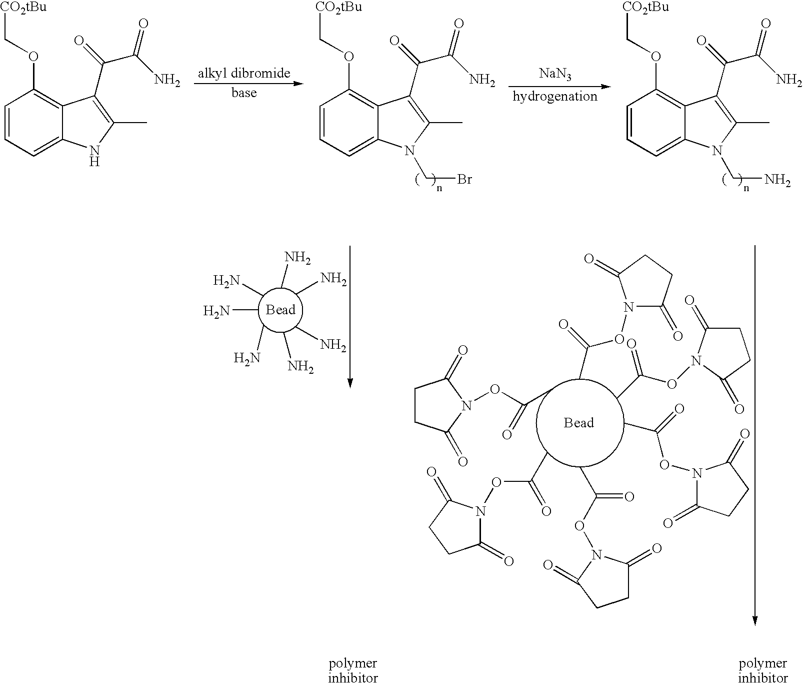

In a second approach, common monomers are copolymerized with an inhibitor bearing a polymerizable linker. This approach provides random copolymer, or it can provide a block copolymer when living polymerization technique is applied, and alternative, it can provide a crosslinked copolymer when crosslinker is used. With selection of common monomers the material could be hydrophobic, hydrophilic, or their combinations. The inhibitor can be synthesized thru alkylation of indole N1 position as shown in the following scheme:

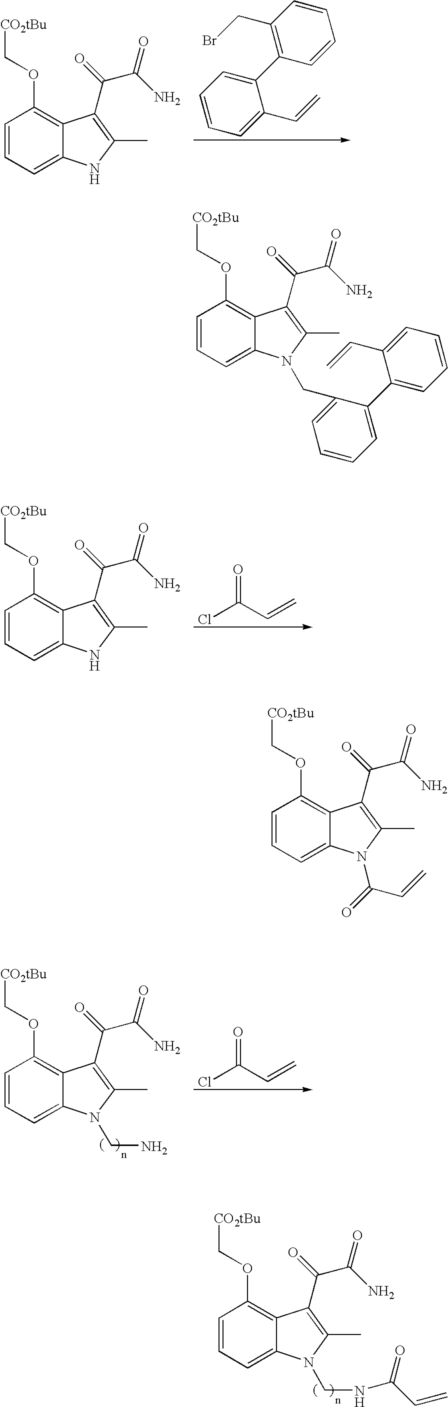

In a third approach, control free radical polymerization can be used to achieve a variety of polymer architectures.

In a first scheme within this third approach, polymer-tailored inhibitors can be prepared. The phospholipase inhibiting moiety bearing a free radical control agent can be synthesized by N1 alkylation with eg. 2-chloro-propionyl chloride or further derivatized to thiourathane. Atom transfer radical polymerization (ATRP) or Reversible addition-fragmentation chain transfer polymerization (RAFT) can be employed to control the chain length of polymer by the ratio of monomers and control agent. The chain end group can be removed by reduction or reserved for dimerization.

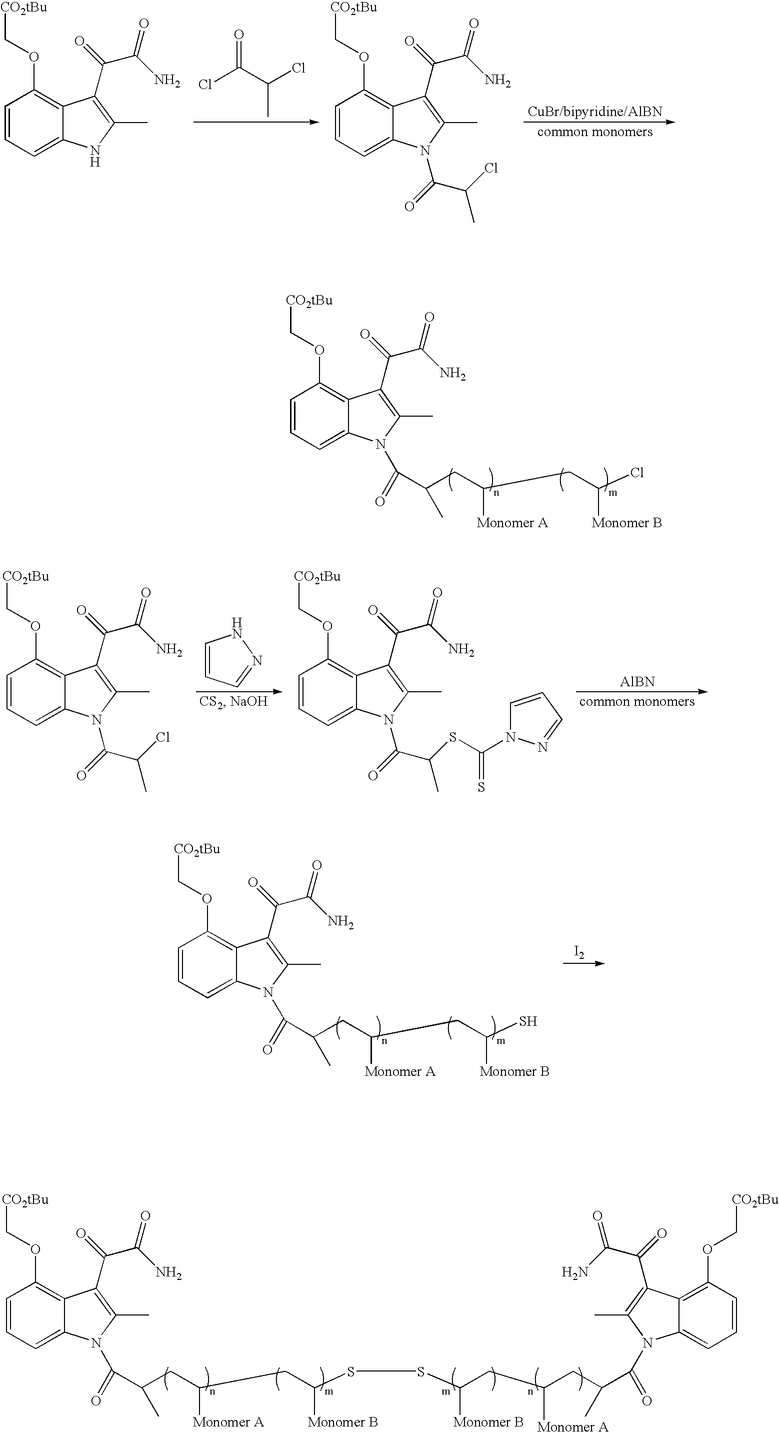

In a second scheme within this third approach, an alternative approach to a short chain inhibitor dimer can be achieved by the route outlined below. Commercial available alkyl dibromide is used as the linker with bromide or thiol end functional group. Then two inhibitor can be jointed by a amine, sulfide, or a disulfide bond. Other jointing functional group also can be applied after derivatization of bromide linker.

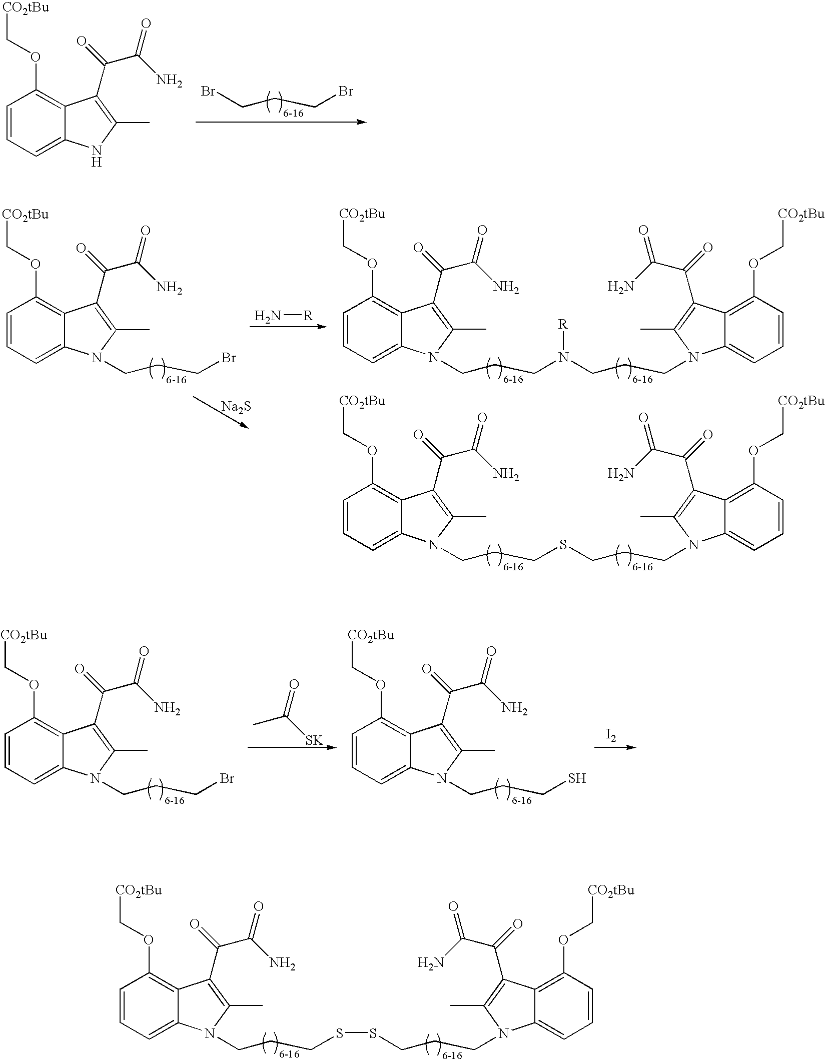

In a third scheme within this third approach involving free-radical polymerization a phospholipase inhibitor-tailored star copolymer can be prepared as follows. The polymer-tailored inhibitor from the first or second schemes within this third approach can be further polymerized with monomers and crosslinker to achieved star copolymer architecture with inhibitor at the chain ends, as shown below:

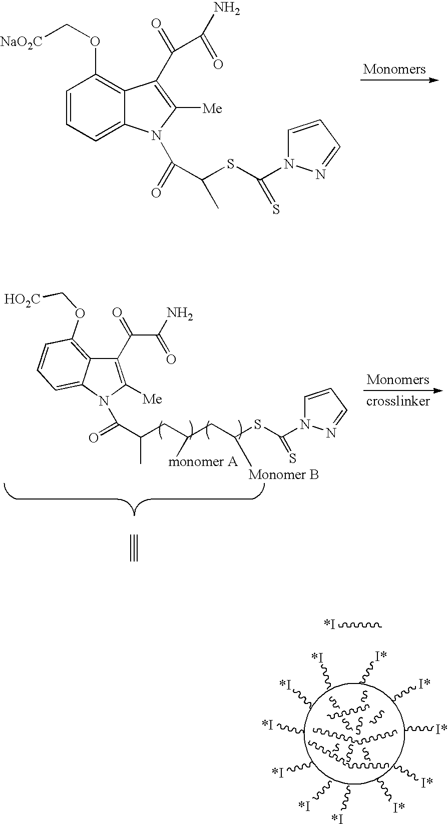

In a fourth scheme within this third approach involving free-radical polymerization, a hyperbranched copolymer can be formed as follows. Copolymerization of control-agent-linked phospholipase inhibitor, AB₂ type monomer, and common monomers provides a hyperbranched copolymer with inhibitor at the chain end as shown below.

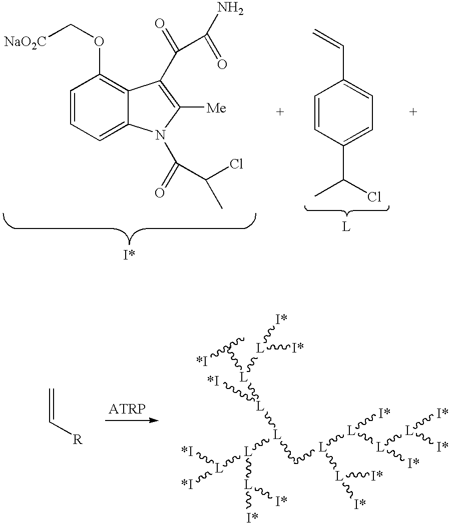

Other art-know phospholipase A2 inhibitors are based on indole compounds or indole-related compounds. (See, for example a summary as shown in co-owned PCT Application No. US/2005/______ entitled “Treatment of Diet-Related Conditions Using Phospholipase-A2 Inhibitors Comprising Indoles and Related Compounds” filed on May 3, 2005 by Buysse et al.), incorporated herein by reference.

Other art-know phospholipase A2 inhibitors (in addition to the indole and indole-related compounds) are also useful as phospholipase inhibiting moieties of the present invention, and can include the following classes: Alkynoylbenzoic, -Thiophenecarboxylic, -Furancarboxylic, and -Pyridinecarboxylic acids (e.g. see U.S. Pat. No. 5,086,067); Amide carboxylate derivatives (e.g. see WO9108737); Aminoacid esters and amide derivatives (e.g. see WO2002008189); Aminotetrazoles (e.g. see U.S. Pat. No. 5,968,963); Aryoxyacle thiazoles (e.g. see WO00034254); Azetidinones (e.g. see WO9702242); Benzenesulfonic acid derivatives (e.g. see U.S. Pat. No. 5,470,882); Benzoic acid derivatives (e.g. see JP08325154); Benzothiaphenes (e.g. see WO02000641); Benzyl alcohols (e.g. see U.S. Pat. No. 5,124,334); Benzyl phenyl pyrimidines (e.g. see WO00027824); Benzylamines (e.g. see U.S. Pat. No. 5,039,706); Cinnamic acid compounds (e.g. see JP07252187); Cinnamic acid derivatives (e.g. see U.S. Pat. No. 5,578,639); Cyclohepta-indoles (e.g. see WO03016277); Ethaneamine-benzenes; Imidazolidinones, Thiazoldinones and Pyrrolidinones (e.g. see WO03031414); Indole glyoxamides (e.g. see U.S. Pat. No. 5,654,326); Indole glyoxamides (e.g. see WO9956752); Indoles (e.g. see U.S. Pat. No. 6,630,496 and WO9943672; Indoly (e.g. see WO003048122); Indoly containing sulfonamides; N-cyl-N-cinnamoylethylenediamine derivatives (e.g. see WO9603371); Naphyl acateamides (e.g. see EP77927); N-substituted glycines (e.g. see U.S. Pat. No. 5,298,652); Phosopholipid analogs (e.g. see U.S. Pat. No. 5,144,045 and U.S. Pat. No. 6,495,596); piperazines (e.g. see WO03048139); Pyridones and Pyrimidones (e.g. see WO03086400); 6-carbamoylpicolinic acid derivatives (e.g. see JP07224038); Steroids and their cyclic hydrocarbon analogs with amino-containing sidechains (e.g. see WO8702367); Trifluorobutanones (e.g. see U.S. Pat. No. 6,350,892 and US2002068722); Abietic derivatives (e.g. see U.S. Pat. No. 4,948,813); Benzyl phosphinate esters (e.g. see U.S. Pat. No. 5,504,073); each of which is incorporated herein by reference.

Specific examples of phospholipase inhibiting moieties of some of these PL A₂ inhibitor classes are provided in Table 1 below, along with IC50 values corresponding thereto:

| Example of phospholipase inhibiting moiety from a PL A₂ inhibitor class | IC50 |
|---|---|
| Alkynoylbenzoic, -Thiophenecarboxylic, -Furancarboxylic, and | μM range |
| 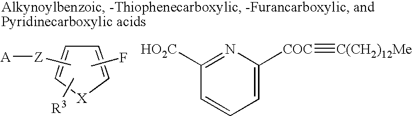 | |
| 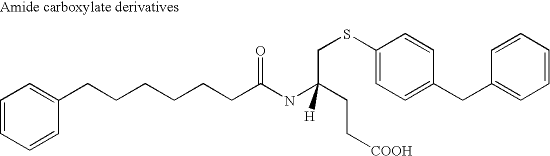 | sub μM range |
| 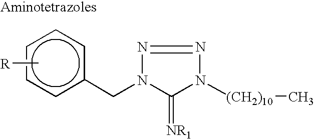 | about 2.5 μM μM range |
| 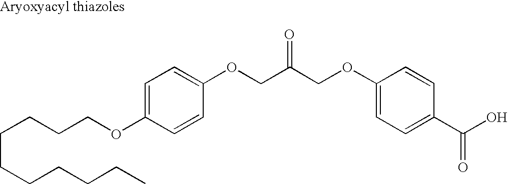 | |
| 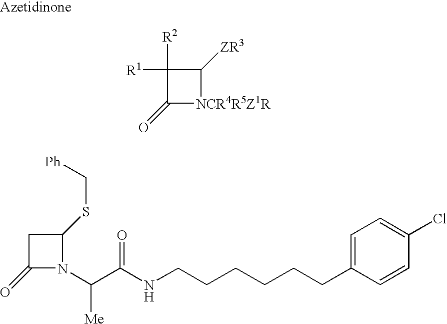 | |
| 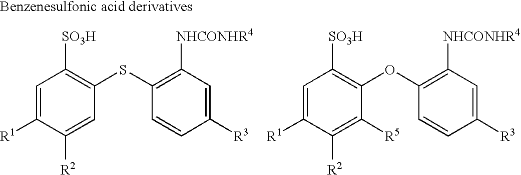 | |
| Benzoic acid derivatives | μM range |
| Benzothiaphenes | about 1.4 μM |
| 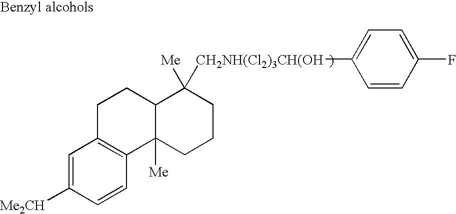 | about 10 μM |
| Benzyl phenyl pyrimidines | |
| 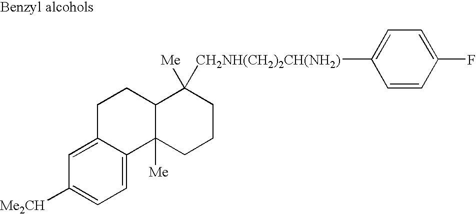 | μM range |
| Cinammic acid compounds | about 70 nM |
| 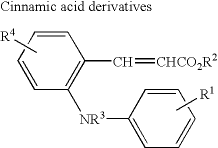 | μM range |
| Cyclohepta-indoles, e.g., preclinical candidate LY-311727 | |
| 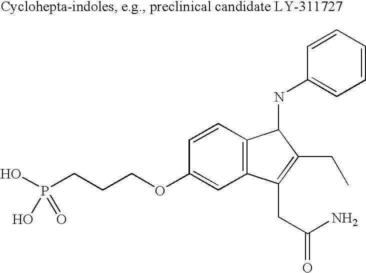 | sub μM range |
| 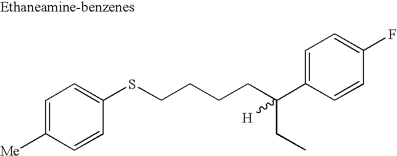 | μM range |
| Imidazolidinones, thiazolidinones and pyrrolidinones | |
| 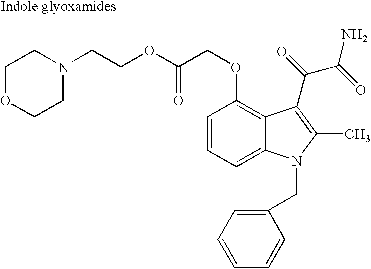 | |
| Indoles | about 0.08 μM to about |
| | 50 μM |
| 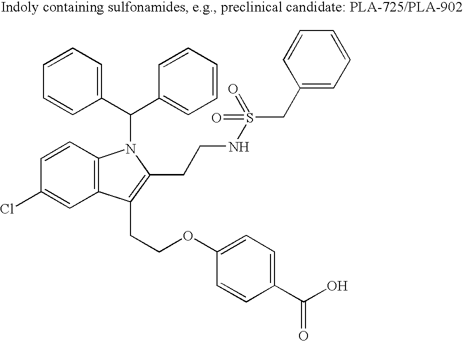 | |
| 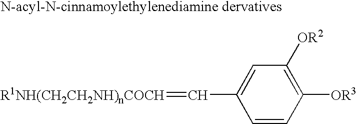 | about 7 μg/mL |
| 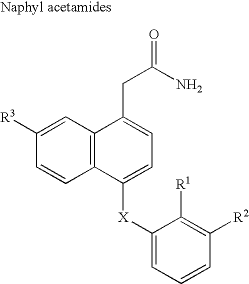 | about 0.87 nμM |
| 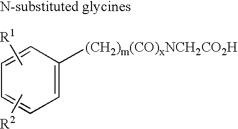 | μM range |
| 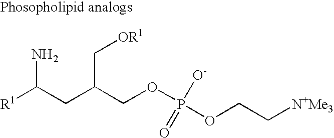 | μM range |
| Piperazines | μM range |
| 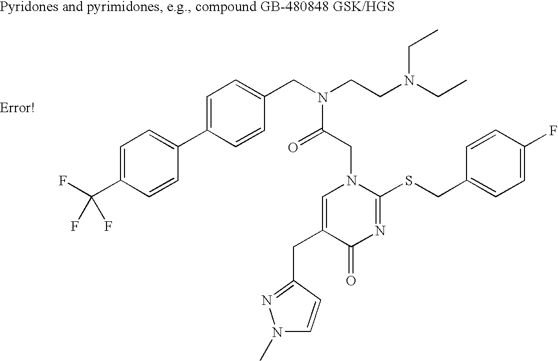 | nM or subnM range |
| 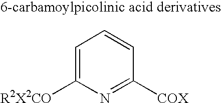 | μM range |
| 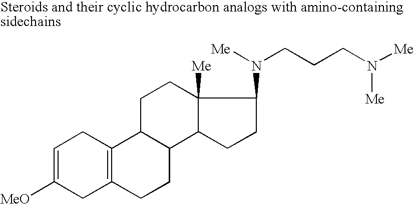 | sub μM range |
| 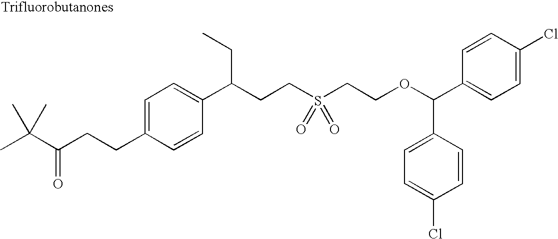 | about 1 μM to about 50 μM |
| 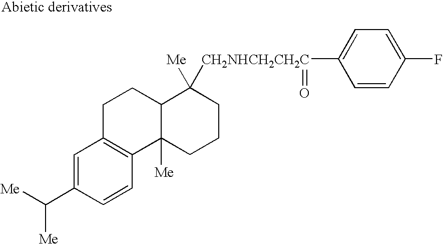 | μM range |
| 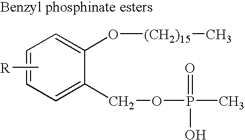 | μM range |
| 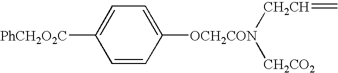 | μM range |

Phospholipase inhibiting moieties useful in some phospholipase inhibitors of the present invention also include natural products, such as Manoalide, a marine product extracted from the sponge Luffariella variabilis, as well as compounds related thereto, illustrated along with the structure of Manoalide below:

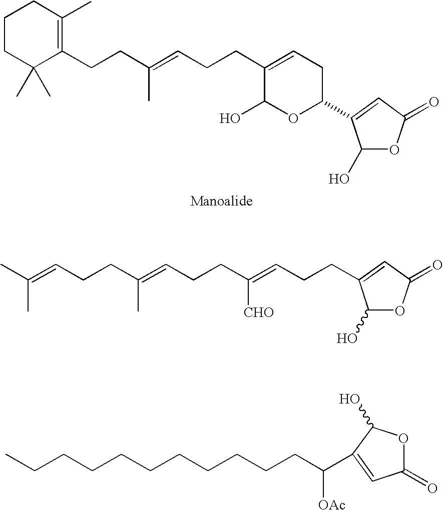

Any of these compounds can be used as a phospholipase inhibiting moiety of the non-absorbed inhibitors in some embodiments of the present invention. As described in more derail above, such moieties may have particular mass, charge and/or other physical parameters to hinder (net) absorption through a gastrointestinal tract, and/or can be linked to a non-absorbed moiety, e.g., a polymer moiety. Furthermore, the invention is not limited to the compositions disclosed herein. Other compositions useful in the present invention would be apparent to one of skill in the art, based on the teachings presented herein, and are also contemplated as within the scope of the invention.

The point of attachment of a phospholipase inhibiting moiety to a non-absorbed moiety, e.g., a polymer moiety, can be selected so as not to interfere with the inhibitory action of the phospholipase inhibiting moiety, e.g., its ability to blunt or reduce the catalytic activity of PL A₂. For instance when a phospholipid analog is used as Z, minimal loss of activity can be achieved by attaching the linking moiety to the hydrophobic group of the phospholipid analog (e.g., its long chain alkyl group) rather than, for example, to its polar head group. Without being limited to a particular hypothesis, phospholipid analogs can inhibit PL A₂ by competing with phospholipid substrates for the catalytic site, which recognizes the polar head group rather than the hydrophobic group of the phospholipid substrate or phospholipid analog. Thus, attachment to the weakly-recognized hydrophobic group can minimize interference with enzyme inhibitory activity of the phospholipid analog. Those of skill in the art will recognize other suitable attachment points for other art-known phospholipase inhibiting moieties.

For example, suitable points of attachment can be identified by available structural information. A co-crystal structure of a phospholipase inhibiting moiety bound to a phospholipase allows one to select one or more sites where attachment of a linking moiety would not preclude the interaction between the phospholipase inhibiting moiety and its target. For instance, preferred points of attachment of phospholipase inhibiting moieties selected from various classes of art-known phospholipase inhibitors are indicated with arrows below:

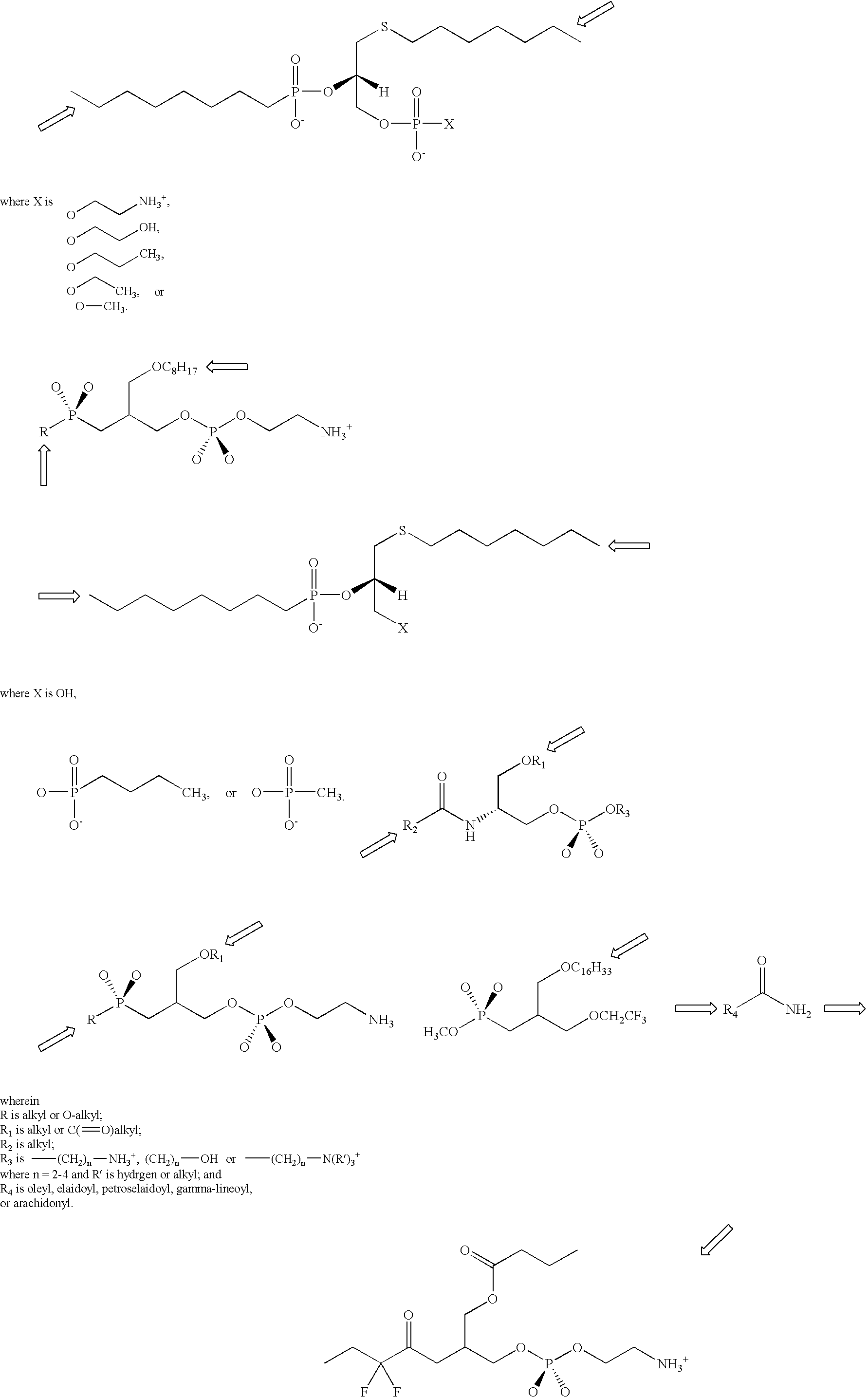

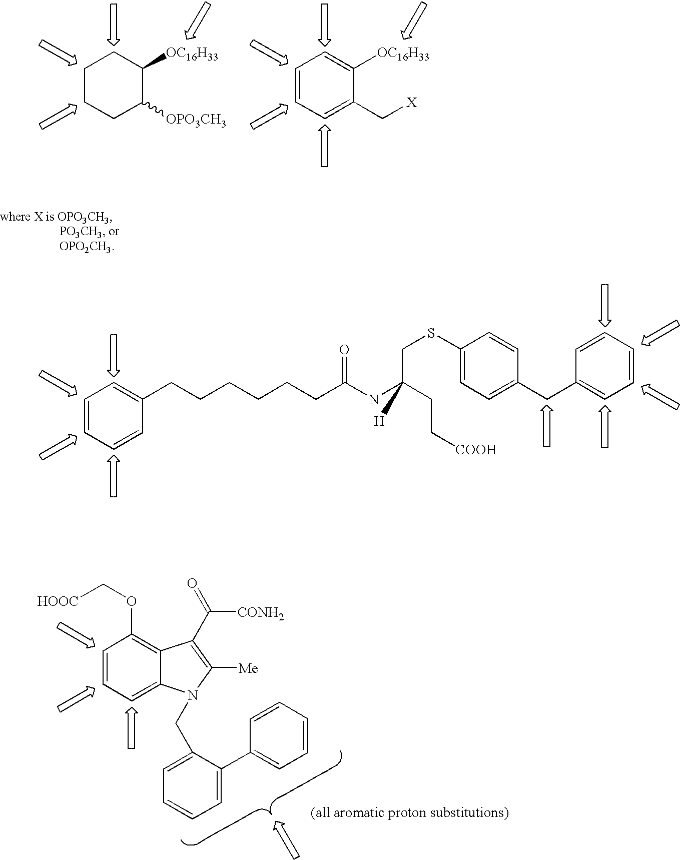

Further, evaluation of binding of a phospholipase inhibitor to a phospholipase by nuclear magnetic resonance permits identification of sites non-essential for such binding interaction. Additionally, one of skill in the art can use available structure-activity relationship (SAR) for phospholipase inhibitors that suggest positions where structural variations are allowed. A library of candidate phospholipase inhibitors can be designed to feature different points of attachment of the phospholipase inhibiting moiety, e.g., chosen based on information described above as well as randomly, so as to present the phospholipase inhibiting moiety in multiple distinct orientations. Candidates can be evaluated for phospholipase inhibiting activity, as discussed in more detail below, to obtain phospholipase inhibitors with suitable attachment points of the phospholipase inhibiting moiety to the polymer moiety or other non-absorbed moiety.

In a third general embodiment, a phospholipase inhibitor can comprises a small organic molecule. As noted above in connection with the inhibitor moiety of the second general embodiment, a small molecule inhibiting moiety that is lumen-localized can comprise a moiety derived from a substituted organic compound having a fused five-member ring and six-member ring, and preferably a fused five-member ring and six-member ring having one or more heteroatoms (e.g., nitrogen, oxygen) substituted within the ring structure of the five-member ring, within the ring structure of the six-member ring, or within the ring structure of each of the five-member and six-member rings. In each case the inhibiting moiety can comprise substituent groups effective for imparting phospholipase inhibiting functionality to the moiety. Reference is made to the previous discussion above with respect to preferred compounds having fused five-member and six-member rings.

In preferred embodiments, a small molecule phospholipase inhibitor can comprise an indole, such as a substituted indole. Reference is made to the previous discussion above with respect to preferred indole-based compounds.

One small molecule organic compound, ILY-4001, which is represented by the structure: 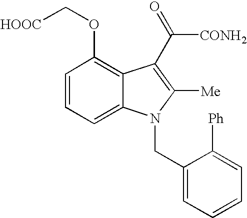
was synthesized (See for example, Example 1A) and evaluated for bioavailability (See, for example, Example 1B). Bioavailability can be reduced (reciprocally, lumen-localization can be improved) according to this third general embodiment of the invention, for example, by charge-modifying strategies applied to this indole moiety to a polymer. (See, for example, Example 1C).

With respect to chemistry for charge modification, general chemistry to indole derivatives is known in literature for example: J. Med. Chem. 1996, 39, 5119-5136.; J. Med. Chem. 1996, 39, 5137-5159.; J. Med. Chem. 1996, 39, 5159-5175. Chemistry approaches to increase charge moiety on indole derivatives for non-absorbability includes modification of indole C4′, C5, C6, C7, and N1 positions (FIG. 5) with polar groups such as carboxylic, sulfonate, sulfate, phosphonate, phosphate, amine, etc. as an example indole C5 modification uses the commercial available 4-hydroxy indole as a starting material. After selective mild base alkylation on 4-hydroxy position with allyl bromide the 2-phenyl benzyl group is installed at N position using sodium hydride as a base. The standard glyoxamidation is then followed. The subsequent Claisen rearrangement and alkylation of tert-butyl protected acetate give the intermediate with C5 alkyl substitution for further polar group installation.

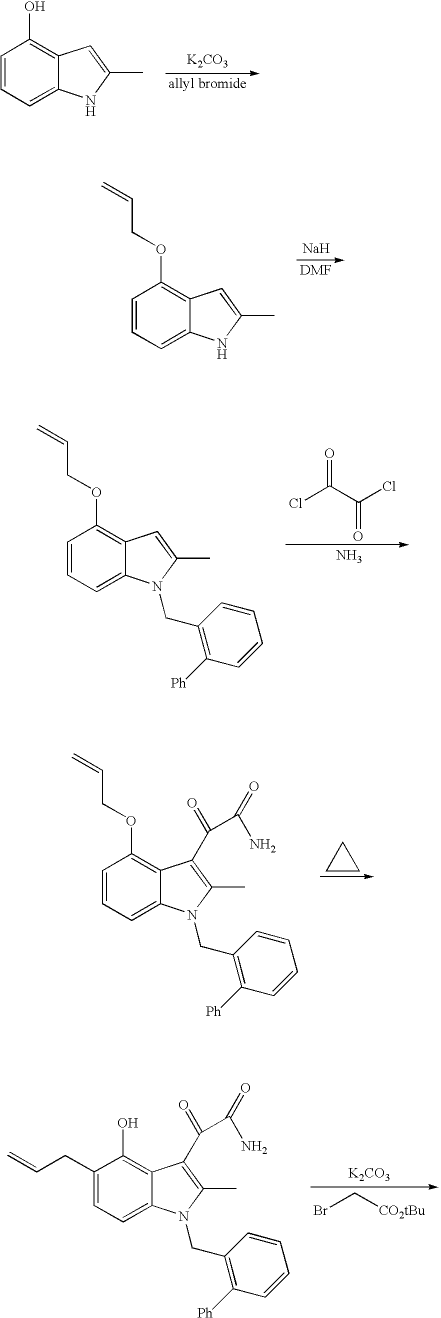

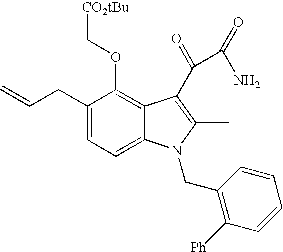

The C5 allyl intermediate is versatile in the sense that not only provides an access to a variety of polar groups but also can modulate length of the group for the SAR study. For example in Pathway A, the target molecule can be obtained via olefin isomerization, ozonolysis, and followed by oxidation to give C5 formic acid derivative. In Pathway B, the allyl intermediate is converted to the corresponding diol by dihydroxylation, then followed by periodate cleavage to afford the aldehyde. Further oxidation of the aldehyde to give acetic acid derivative, or reduction of aldehyde to the corresponding hydroxyl intermediate for further transformation to amine, sulfonate, and phosphonate. In pathway C, the propionic acid derivative can be obtained via hydroboration of olefin and following by oxidation of the corresponding alcohol. In pathway D, the allyl intermediate could simply undergo aminohydroxylation to afford the target. 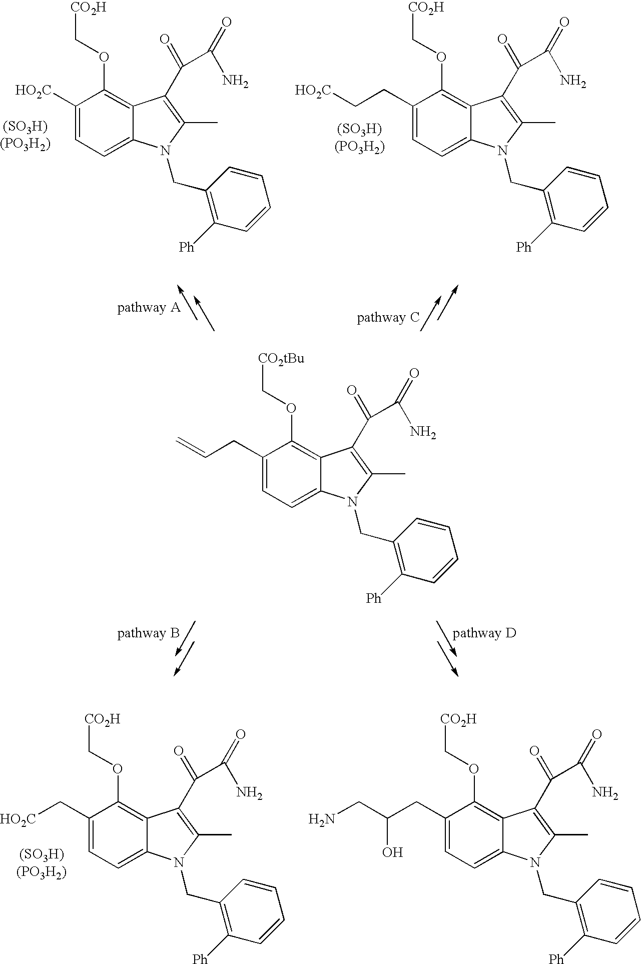
Localization in the Gastrointestinal Lumen Via Efflux

In some embodiments a phospholipase inhibitor is constructed to hinder its (net) absorption through a gastrointestinal mucosa and/or comprises a phospholipase inhibiting moiety linked, coupled or otherwise attached to a non-absorbed moiety as described above. In some embodiments, the phospholipase inhibitor is localized in a gastrointestinal lumen due to efflux. In some embodiments, the inhibitor is effluxed from a gastrointestinal mucosal cell, for example, an intestinal and/or a colonic enterocyte, upon entry into the cell, creating the net effect of non-absorption. Any art-known phospholipase inhibitor and/or any phospholipase inhibiting moiety described and/or contemplated herein can be used in these embodiments. For example, any art known PL A₂ inhibitors provided in Table 1 can be used. These and other art-known phospholipase inhibitors and/or any phospholipase inhibiting moiety disclosed and/or contemplated herein can be constructed to be effluxed back into a gastrointestinal lumen upon movement therefrom.

In some efflux embodiments, the phospholipase inhibitor remains localized in the gastrointestinal lumen even though it may be absorbed by a gastrointestinal mucosal cell by active and/or passive transport, or otherwise permeate through the gastrointestinal wall by active and/or passive transport. The phospholipase inhibitor in some embodiments may have one or more hydrophobic and/or lipophilic moieties, tending to allow diffusion across the plasma membrane of a gastrointestinal mucosal cell. However, subsequent passage across the basolateral membrane and into the portal blood circulation can be regulated by a number of physical and molecular considerations, discussed in detail below. For example, a phospholipase inhibitor that enters an intestinal and/or a colonic enterocyte, e.g., an apical enterocyte, can be subsequently effluxed back into the gastrointestinal lumen.

In some embodiments, efflux is achieved by protein and/or glycoprotein transporters located in a gastrointestinal mucosal cell, for example, in an apical enterocyte of the gastrointestinal tract. Protein and/or glycoprotein transporters include, but are not limited to, for example, ATP-binding cassette transport proteins, such as P-glycoproteins including MDR1 (product of ABCB1 locus) and MRP2, located in the epithelial cells of the gut, for example, in the apical enterocytes of the gastrointestinal tract. Such transports may also be referred to pumps.

In some embodiments, for example, a phospholipase inhibitor can be constructed so as to be recognized by a protein and/or glycoprotein transporter that effluxes the inhibitor from the cytoplasm of an enterocyte back into the gastrointestinal lumen. In some embodiments, the phospholipase inhibitor is constructed so as to allow intracellular modification, e.g., via metabolic processes, within the enterocyte to facilitate recognition by a protein and/or glycoprotein transporter, such that the modified inhibitor serves as a target for transport. Motifs that are recognized by protein and/or glycoprotein transporters of the gut epithelium can be determined by one of ordinary skill in the art. For example, recognition motifs for ATP-binding cassette transport proteins, such as P-glycoproteins including MDR1 (product of ABCB1 locus) and MRP2 can be determined. A phospholipase inhibitor of the present invention may comprise a phospholipase inhibiting moiety linked, coupled, or otherwise attached to a recognition motif moiety. “Recognition motif moiety” as used herein refers to a moiety comprising a motif that is recognized by a transporter, or than can be modified to become recognized by a transporter, where the transporter can effect efflux of a composition comprising the recognition motif moiety into the gastrointestinal lumen, including, for example motifs recognized by protein and/or glycoprotein transporters of the gut epithelium such as ATP-binding cassette transport proteins, P-glycoproteins, MDR1, MRP2, and the like. In some embodiments, the recognition motif moiety serves as a target for a transporter of a gut epithelial cell, causing the transporter to drive the phospholipase inhibitor from the inside of the cell back into the gastrointestinal lumen. Lumen localization achieved by efflux can thus hinder or prevent absorption of the phospholipase inhibitor into the blood circulation.

In preferred embodiments, efflux achieves lumen localization of a significant amount, preferably a statistically significant amount, and more preferably essentially all, of the phospholipase inhibitor introduced into the gastrointestinal lumen. That is, essentially all of the phospholipase inhibitor remains in the gastrointestinal lumen by efflux of some, most, and/or essentially all of any inhibitor that moves out of the gastrointestinal lumen. For example, the effect can be such that at least about 90% of phospholipase inhibitor remains in the gastrointestinal lumen, at least about 95%, at least about 98%, preferably at least about 99%, and more preferably at least about 99.5% remains in the gastrointestinal lumen.

In some embodiments, the phospholipase inhibitor comprises one or more additional efflux enhancing moieties. “Efflux enhancing moiety” as used herein refers to a moiety comprising an efflux enhancer that acts to enhance, aid, increase, activate, promote, or otherwise facilitate efflux of the moiety into the gastrointestinal lumen. For example, the phospholipase inhibitor in some embodiments may comprise a moiety that activates expression of a transporter, for example, a transcription factor and/or an enhancer of a gene encoding a transporter. For example, the nuclear receptor, pregnane X, also referred to as the pregnane X receptor (PXR), induces high levels of MDR1 and/or related transporters. (CITE). In some preferred embodiments, the phospholipase inhibitor is coupled, linked and/or otherwise attached to an efflux enhancing moiety that activates PXR, e.g., by contacting and binding to the nuclear receptor. The higher levels of MDR1 and/or related transporters produced can enhance efflux of phospholipase inhibitor that also comprises, for example, a recognition motif for MDR1. Based on the teachings herein, those of ordinary skill in the art will recognize other efflux enhancing moieties that may be used in these aspects of the invention, and which are also contemplated within its scope.

Some embodiments of the present invention involve a combination of non-absorbed and effluxed inhibitors. In such embodiments, lumen localization is achieved by a combination of non-absorption of the phospholipase inhibitor and efflux of some, most, and/or essentially all of any phospholipase inhibitor that moves out of the gastrointestinal lumen.

Lumen-localization can improve the potency of the phospholipase inhibitor, so that the amount of inhibitor administered can be less than the amount administered in the absence of non-absorption and/or efflux. In some embodiments, non-absorption and/or efflux improves the efficacy of the phospholipase inhibitor. In particular, the inhibitor reduces the activity of phospholipase to a greater extent when localized in the lumen by non-absorption and/or efflux. In such embodiments, the amount of phospholipase inhibitor used can be the same as the recommended dosage levels or higher than this dose or lower than the recommended dose. In some embodiments, non-absorption and/or efflux decreases the dose of phospholipase inhibitor used and thus can increase patient compliance and decrease side-effects.

Phospholipase Inhibition by Lumen-Localized Phospholipase Inhibitors

In addition to lumen-localization functionality, the phospholipase inhibitors of the invention should also have an enzyme-inhibiting functionality.

Generally, the term “inhibits” and its grammatical variations are not intended to require a complete inhibition of enzymatic activity. For example, it can refer to a reduction in enzymatic activity by at least about 50%, at least about 75%, preferably by at least about 90%, more preferably at least about 98%, and even more preferably at least about 99% of the activity of the enzyme in the absence of the inhibitor. Most preferably, it refers to a reduction in enzyme activity by an effective amount that is by an amount sufficient to produce a therapeutic and/or a prophylactic benefit in at least one condition being treated in a subject receiving phospholipase inhibiting treatment, e.g., as disclosed herein. Conversely, the phrase “does not inhibit” and its grammatical variations does not require a complete lack of effect on the enzymatic activity. For example, it refers to situations where there is less than about 20%, less than about 10%, less than about 5%, preferably less than about 2%, and more preferably less than about 1% of reduction in enzyme activity in the presence of the inhibitor. Most preferably, it refers to a minimal reduction in enzyme activity such that a noticeable effect is not observed. Further, the phrase “does not significantly inhibit” and its grammatical variations refers to situations where there is less than about 40%, less than about 30%, less than about 25%, preferably less than about 20%, and more preferably less than about 15% of reduction in enzyme activity in the presence of the inhibitor. Further, the phrase “essentially does not inhibit” and its grammatical variations refers to situations where there is less than about 30%, less than about 25%, less than about 20%, preferably less than about 15%, and more preferably less than about 10% of reduction in enzyme activity in the presence of the inhibitor.

In some embodiments, a phospholipase inhibitor of the present invention acts to inhibit phospholipase such as phospholipase A₂ by hindering access of the enzyme to its phospholipid substrate; in some embodiments it acts by reducing the enzyme's catalytic activity with respect to its substrate; in some embodiments the phospholipase inhibitor acts by a combination of these two approaches.

As discussed above, some gastrointestinal phospholipases, e.g., most PL A₂ enzymes, act on their substrates while physically proximate to (e.g., “docked”) to a lipid-water interface of a lipid aggregate. As such, catalytic activity can depend at least in part on the enzyme having physical access to the outer surface of lipid aggregates in the gastrointestinal lumen. With reference to the schematic, non-limiting representation illustrated in FIG. 1A, for example, a PL A₂ enzyme 10 can interact with a lipid-water interface 22 of a lipid aggregate 20. The catalytic site 12 of the i-face of the enzyme is depicted by a “notch” on the face that interacts with the lipid aggregate 20.

In some embodiments of the present invention, PL A₂ inhibition is achieved by keeping the enzyme off the outer surface of lipid aggregates, thereby hindering access to phospholipid substrates. FIGS. 1B and 1C illustrate two embodiments of non-absorbed polymeric phospholipase inhibitors that can inhibit enzyme activity by hindering access of the enzyme to a phospholipid substrate at a lipid-water interface. Specifically, referring to FIG. 1B, a non-absorbed phospholipase inhibitor 30 consisting essentially of a polymer moiety having hydrophobic end-regions 32 associates with a lipid-water interface 22, and hinders accessibility of the enzyme 10 to the lipid-water interface 22. FIG. 1C illustrates a non-absorbed phospholipase inhibitor 30 consisting essentially of a polymer interacting with the phospholipase enzyme 10, and hindering accessibility of the enzyme 10 to the lipid-water interface 22. The non-absorbed phospholipase inhibitor 30, consisting essentially of polymer having hydrophobic end-regions 32, can associate with both the phospholipase enzyme 10 and a lipid-water interface 22, as illustrated in FIG. 1D.

A non-absorbed inhibitor that acts by hindering access need not directly interfere with the catalytic site of the enzyme, for example, it need not recognize and/or bind to the enzyme's catalytic site or to any other specific site on the enzyme, such as an allosteric site. Rather, in some embodiments, a non-absorbed phospholipase inhibitor of the present invention may prevent or hinder physical adsorption of the enzyme at a lipid-water interface of one or more types of lipid aggregates found in the gastrointestinal lumen. Examples of a “lipid-water interface” include the outer surface of a lipid aggregate found in the gastrointestinal lumen, including, for example, a fat globule, an emulsion droplet, a vesicle, a mixed micelle, and/or a disk, any one of which may contain triglycerides, fatty acids, bile acids, phospholipids, phosphatidylcholine, lysophospholipids, lysophosphatidylcholine, cholesterol, cholesterol esters, other amphiphiles and/or other diet metabolites.

In preferred embodiments, the inhibitor comprises a polymer moiety capable of interacting with either a phospholipase and/or the lipid-water interface of a lipid aggregate. FIG. 1B illustrates an example where the inhibitor 30 interacts with a lipid-water interface 22 such that it becomes physically complexed, coupled, bound, attached, or otherwise adsorbed to the lipid-water interface 22. The inhibitor 30 can interact with the interface 22 through any bonding interaction, including, for example, covalent, ionic, metallic, hydrogen, hydrophobic, and/or van der Waals bonds, preferably hydrophobic an/or ionic bonds. In the example of FIG. 1B inhibitor interaction with a lipid-water interface 22 is facilitated by hydrophobic bonds. In this depicted embodiment, the inhibitor has two end-regions 32 each of which bears a hydrophopic moiety (depicted by solid rectangles), e.g., phospholipid analogs, that become embedded in the lipid layer via hydrophobic interactions between the moieties of the inhibitor 30 and the hydrophobic chains of the bilayer.

FIG. 1C illustrates an example where the inhibitor 30 interacts with a phospholipase enzyme 10, e.g. PL A₂. In some embodiments, the phospholipase inhibitor 30 comprises a moiety that becomes physically complexed, coupled, bound, attached, or otherwise adsorbed to the enzyme 10 so as to hinder its interaction with a lipid aggregate 20. The inhibitor 30 can be described as scavenging the enzyme in solution to create a complex with it. In some embodiments, the enzyme 10 interacting with the inhibitor 30 is sterically hindered from access to its phospholipid substrate at a lipid-water interface 22, for example, because its approach to the interface 22 is physically hindered.

In some embodiments, the inhibitor comprises a polymer moiety that can be soluble or insoluble under the physiological conditions of the gastrointestinal lumen, and may exist, for example, as dispersed micelles or particles, such as colloidal particles or (insoluble) macroscopic beads, as described in detail above. With reference to FIG. 2, for example, phospholipase inhibitors 30, including both soluble and insoluble inhibitors 30, can comprising polymer moieties covalently linked to phospholipase inhibiting moieties (represented schematically by “I*”). The phospholipase inhibitors 30 can interact with the phospholipase-A₂ 10 in a gastrointestinal fluid, for example, in the vicinity of gastrointestinal lipid vesicles.

Referring now to FIGS. 3A through 3B, for example, the inhibitor 30 comprises a polymer moiety covalently linked to a single inhibiting moiety (represented schematically by I*) as a singlet embodiment or to two inhibiting moieties as a dimer embodiment (in each case as described above). In FIG. 3A, the phospholipase inhibitor 30 comprises a hydrophobic polymer moiety, adapted such that the inhibitor 30 associates with a lipid-water interface 22 of a lipid vesicle 20 (shown with the hydrophobic polymer moiety being substantially integral with the lipid bilayer). In FIG. 3B, the phospholipase inhibitor 30 comprises a polymer moiety having a first hydrophobic block and a second hydrophilic block with the second hydrophilic block being proximal to the phospholipase inhibiting moiety, and adapted such that the inhibitor 30 associates with a lipid-water interface 22 of a lipid vesicle 20 (shown with the hydrophobic block being substantially integral with the lipid bilayer and with the hydrophilic block being substantially associated within the aqueous phase surrounding the lipid bilayer). Referring to FIG. 3C, the phospholipase inhibitor 30 comprises a hydrophobic polymer moiety covalently linked to two inhibiting moieties, and adapted such that the inhibitor 30 associates with a lipid-water interface of a lipid vesicle 20 (shown with the hydrophobic polymer moiety being substantially integral with and looped through the lipid bilayer. These embodiments allow for interaction between the inhibiting moiety and phospholipase-A₂ substantially proximate to the vesicle surface.

Generally, in any aspect or embodiment of the invention requiring a polymer moiety, the polymer moiety of the inhibitor can be shaped in various formats, preferably designed to favor the formation of a complex with a phospholipase, e.g., a complex with PL A₂. For instance, the polymer moiety may comprise a macromolecular scaffold designed to interact with the i-face of PL A₂. As discussed above, the structural features of the i-face are such that the aperture of the slot forming the catalytic site is normal to the i-face plane. The aperture is surrounded by a first crown of hydrophobic residues (mainly leucine and isoleucine residues), which itself is contained in a ring of cationic residues, (including lysine and arginine residues). The polymer moiety may be designed as a macromolecular scaffold comprising a plurality of anionic moieties (e.g., arranged so as to bind to the cationic ring) and/or a plurality of hydrophobic residues (e.g., arranged so as to bind to the hydrophobic crown). In such embodiments, the inhibitor becomes positioned over the catalytic site bearing face of a phospholipase and hinders access to the catalytic site as a “lid” or “cap” blocks access to an aperture.

As described above, the inhibitor can comprises a non-absorbed oligomer or polymer moiety and a phospholipase inhibiting moiety. The phospholipase inhibiting moiety may be coupled, linked or otherwise attached to the non-absorbed moiety. In one embodiment, the inhibiting moiety may be linked, for example, to a polymer moiety that interacts with a lipid-water interface and/or a polymer moiety that interacts with phospholipase. In the latter case, the phospholipase inhibiting moiety may further aid the interaction of the polymer moiety with the phospholipase, e.g., with the i-face of PL A₂.

In some embodiments, for example, a PL A₂ inhibiting moiety is linked, coupled or otherwise attached is coupled to a macromolecular scaffold of a polymer moiety where the PL A₂ inhibiting moiety interacts with the catalytic site of PL A₂ while the macromolecular scaffold interacts with the i-face surrounding the catalytic site. Where the phospholipase inhibiting moiety comprises a phospholipid analog or a transition state analog, the phospholipase inhibiting moiety is preferably coupled via its hydrophobic group, leaving the polar head group of the inhibiting moiety available for binding to the catalytic site, e.g., through the His-calcium-Asp triad discussed above.

Some embodiments comprising a phospholipase inhibiting moiety coupled to a polymer moiety that interacts with a phospholipase comprise a plurality of anionic moieties (e.g., arranged so as to bind to a cationic ring) linked to a spacer moiety (e.g., arranged so as to overlay a hydrophobic crown), which converge on a central or focal point bearing the phospholipase inhibiting moiety. Some such embodiments can be represented by the formula (D) 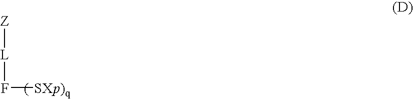
where Z is a phospholipase inhibiting moiety, preferably a PL A₂ inhibiting moiety; L is a linking moiety, e.g., a chemical linker; F is focal point where covalent linkages from a plurality of segments SXp converge; S is a spacer moiety; X is an anionic moiety, preferably an acidic group, for example, but not limited to, a carboxylate group, a sulfonate group, a sulfate group, a sulfamate group, a phosphoramidate group, a phosphate group, a phosphonate group, a phosphinate group, a gluconate group, and the like; and p and q are each integers, preferably where p equals 1, 2, 3, or 4, and preferably where q equals 2, 3, 4, 5, 6, 7, or 8.

The F-(SXp)q segment can adopt various configurations, preferably configurations that facilitate interaction with the catalytic site bearing face of a phospholipase. In some embodiments, for example, a plurality of spacer moieties radiate from the focal point F, which lies at a center of a macromolecular scaffold of the polymer moiety;

In some preferred embodiments, the spacer moiety S provides a plurality of hydrophobic residues, e.g., arranged so as to bind to the hydrophobic crown of the i-face of PL A₂; in some preferred embodiments, the anionic moieties X are arranged so as to bind to the cationic ring of the i-face of PL A₂. Some embodiments comprise a dendritic macromolecular scaffold with spacer moieties branching and diverging from the focal point F. Examples of some embodiments can be represented by the structures provided below:

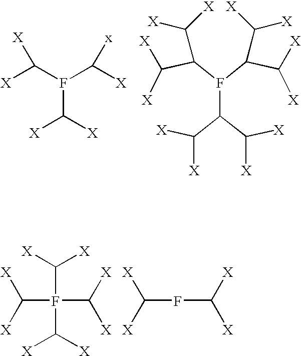

Other examples of dendritic structures useful in the practice of the present invention are known in the art, e.g., see Grayson S. M. et al. Chemical Reviews, 2001, 101: 3819-3867; and Bosman A. W. et al, Chemical Reviews, 1999, 99; 1665-1688, incorporated herein by reference. Additionally, other examples suitable for use in the present invention will be appreciated by those of ordinary skill in the art in light of the disclosures provided herein, and are contemplated as within the scope of this invention.

In some embodiments, the macromolecular scaffold of the polymer moiety can form particles. In such embodiments, a phospholipase inhibiting moiety is preferably coupled to the outer surfaces of such particles. Where the phospholipase inhibiting moiety is a phospholipid analog or transition state analog, the phospholipase inhibiting moiety is preferably linked through its hydrophobic group, as discussed above. The particles so formed may be porous or non-porous, and may be of any shape, such as spherical, elliptical, globular, or irregularly-shaped particles, as discussed in more detail above. The particles can be composed of one or more organic or inorganic polymers moieties, including any of the polymers disclosed herein. In preferred particle embodiments, the particle surface is hydrophobic in nature, carrying acidic groups, X as defined above.

In other embodiments where non-absorbed phospholipase inhibitors comprise a moiety interacting with a specific site on a phospholipase, e.g., the catalytic site of PL A₂, the inhibitor need not prevent access of the enzyme to its substrate, but may act by reducing the enzyme's ability to act on its substrate even if the enzyme approaches and/or becomes “docked” to a lipid-water interface containing the substrate. Such inhibitor embodiments preferably comprise a polymer moiety and one or more phospholipase inhibiting moieties, e.g., an art-known phospholipase inhibitor and/or any phospholipase inhibitor described and/or contemplated herein. Without being bound to a particular hypothesis, for example, such inhibitors can act to reduce phospholipase activity by reversible and/or irreversible inhibition.

Reversible inhibition by a phospholipase inhibitor of the present invention may be competitive (e.g. where the inhibitor binds to the catalytic site of a phospholipase), noncompetitive (e.g., where the inhibitor binds to an allosteric site of a phospholipase to effect an allosteric change), and/or uncompetitive (where the inhibitor binds to a complex between a phospholipase and its substrate). Inhibition may also be irreversible, where the phospholipase inhibitor remains bound, or significantly remains bound, or essentially remains bound to a site on a phospholipase without dissociating, without significantly dissociating, or essentially without dissociating from the enzyme.

As discussed above, PL A₂ enzymes share a conserved active site architecture and a catalytic mechanism involving concerted binding of His and Asp residues to water molecules and a calcium cation. Phospholipid substrate can access the catalytic site by its polar head group through a slot enveloped by hydrophobic and cationic residues. Within the catalytic site, the multi-coordinated calcium ion activates the acyl carbonyl group of the sn-2 position of the phospholipid substrate to bring about hydrolysis. In certain embodiments, PL A₂ inhibiting moieties comprise structures that resemble a phospholipid substrate and/or its transition state.

Without being limited to a particular hypothesis, such moieties can inhibit PL A₂ by competing reversibly with phospholipid substrates for the catalytic site. That is, a structural analog of a phospholipid substrate, preferably, a structural analog of its polar head group and/or a structural analog of a phospholipid substrate transition state can reversibly bind the catalytic site, inhibiting access of the phospholipid substrate. Further, as described in detail above, analog phospholipase inhibiting moieties can be attached to a non-absorbed moiety, e.g., a polymer moiety, at an attachment point that does not interfere with the ability of the analog to bind to the catalytic site, minimizing the inhibitory activity of the analog.

In view of the substantial structure-activity-relationship studies for phospholipase-A2 enzymes, considered together with the significant experimental data demonstrated in Example 5 (including Examples 5A through 5C), a skilled person can appreciate that the observed inhibitive effect of ILY-4001 can be realized in other indole compounds of the invention (having the identical core structure) as well as in indole-related compounds comprising a fused five-membered ring and six-membered ring. In particular, without being bound by theory not expressly recited in the claims, a skilled person can appreciate, with reference to FIG. 6A, for example, that substituents at positions 3 and 4 and 5 of the indole structure can be selected and evaluated to be effective for polar interaction with the enzyme and with calcium ion (associated with the calcium-dependent phospholipase activity). Similarly, a person of skill in the art can appreciate that the substituents at positions 1 and 2 of the indole structure can be selected and evaluated to be relatively hydrophobic. Considered in combination, the polar groups at positions 3, 4 and 5 and the relatively hydrophobic groups at positions 1 and 2 can effectively associate the inhibitor (or inhibiting moiety) with a hydrophilic lipid-water interface (via the hydrophobic regions), and also orient the inhibitor (or inhibiting moiety) such that its polar region can be effectively positioned into the enzyme pocket—with its polar head group directed through a slot enveloped by hydrophobic and cationic residues. Similarly, with reference to FIG. 6B, for example, one can appreciate that corresponding groups on the indole-related compound shown therein can have the same functionality. Specifically, a person of skill in the art can appreciate that substituents at positions R₃, R₄ and R₅ of the indole-related structure can be selected and evaluated to be effective for polar interaction with the enzyme and with calcium ion, and that the substituents at positions R₁ and R₂ of the indole-related structure can be selected and evaluated to be relatively hydrophobic.

Similarly, with reference to FIGS. 6C and 6D, the above-described inverse indole compounds that are mirror-image analogues of the core structure of the corresponding indole of interest, and the above-described reciprocal indole compounds and reciprocal indole-related compounds that are alternative mirror-image analogues of the core structure of the corresponding indole or related compound can be similarly configured with polar substituents and hydrophobic substituents to provide alternative indole structures and alternative indole-related structures within the scope of the invention.

Moreover, a person skilled in the art can evaluate particular inhibitors within the scope of this invention using known assaying and evaluation approaches. For example, the extent of inhibition of the inhibitors of the invention can be evaluated using in-vitro assays (See, for example, Example 1B-1) and/or in-vivo studies (See, for example, Example 10).

Further, in some of these embodiments, the phospholipase inhibitor reduces re-absorption of secreted phospholipase A2 through the gastrointestinal mucosa.

Screening Assays for Identifying Phospholipase Inhibitors

The differential activities of gastrointestinal phospholipases, in particular phospholipase A2, enables the screening for inhibitory compounds that inhibit a particular phospholipase and that can be used with the practice of this invention to selectively treat insulin-related conditions (e.g., diabetes), weight-related conditions (e.g., obesity), cholesterol-related conditions, or a combination thereof.

Certain approaches of the present invention provide a method of making or identifying a phospholipase inhibitor that is localized in a gastrointestinal lumen involving selecting a moiety that inhibits PL A₂ by contacting a candidate moiety with a PL A₂ enzyme or fragment thereof, preferably a fragment containing the catalytic and/or allosteric site of the enzyme, more preferably including the His and Asp residues of the catalytic site; determining whether the candidate moiety interacts with the PL A₂ or fragment thereof; and using the selected candidate moiety as a phospholipase A2 inhibiting moiety of a phospholipase inhibitor that is localized in a gastrointestinal lumen.

Certain other approaches of the present invention provide a method of making or identifying a phospholipase inhibitor that is localized in a gastrointestinal lumen involving selecting a moiety that inhibits PL A₂ by contacting a candidate moiety with a lipid-water interface of a lipid aggregate or fragment thereof; determining whether the candidate moiety interacts with the interface; and using the selected candidate moiety as a phospholipase A2 inhibiting moiety of a phospholipase inhibitor that is localized in a gastrointestinal lumen.

Certain approaches of the present invention provide a method of making or identifying a phospholipase inhibitor that is localized in a gastrointestinal lumen involving selecting a moiety that inhibits PLB by contacting a candidate moiety with a PLB enzyme or fragment thereof; determining whether the candidate moiety interacts with the PLB or fragment thereof; and using the selected candidate moiety as a phospholipase B inhibiting moiety of a phospholipase inhibitor that is localized in a gastrointestinal lumen.

Certain approaches of the present invention provide a method of making or identifying a phospholipase inhibitor that is localized in a gastrointestinal lumen involving selecting a moiety that preferentially inhibits PL A₂ by contacting a candidate moiety with a PL A₂ enzyme or fragment thereof, preferably a fragment containing the catalytic and/or allosteric site of the enzyme, more preferably including the His and Asp residues of the catalytic site and determining whether the candidate moiety interacts with the PL A₂ or fragment thereof; contacting the candidate with a PLB enzyme or fragment thereof and determining whether the candidate interacts with the PLB or fragment thereof; selecting any candidate that interacts with PL A₂ but does not interact with PLB, does not significantly interact with PLB, or essentially does not interact with PLB; and using the selected candidate moiety as a phospholipase A2 inhibiting moiety of a phospholipase inhibitor that is localized in a gastrointestinal lumen.

Certain other approaches of the present invention provide a method of making or identifying a phospholipase inhibitor that is localized in a gastrointestinal lumen involving selecting a moiety that preferentially inhibits PL A₂ by contacting a candidate with a lipid-water interface of a lipid aggregate or fragment thereof and determining whether the candidate moiety interacts with the interface; contacting the candidate moiety with a PLB enzyme or fragment thereof and determining whether the candidate moiety interacts with the PLB or fragment thereof; selecting any candidate moiety that interacts with the lipid-water interface does not interact with PLB, but does not significantly interact with PLB, or essentially does not interact with PLB, and using the selected candidate moiety as a phospholipase A2 inhibiting moiety of a phospholipase inhibitor that is localized in a gastrointestinal lumen.

A lumen-localized phospholipase inhibitor, for example, comprising a phospholipase inhibiting moiety disclosed herein and/or identified by the procedures taught herein, can be used in animal models to demonstrate, for example, suppression of insulin-related conditions (e.g. diabetes) and/or hypercholesterolemia and/or weight-related conditions. A lumen-localized phospholipase inhibitor showing inhibitory activity in a PL A₂ inhibition assay, in about the sub μM range is preferred. More preferably, such inhibitors show non-absorbedness, for example low permeability, in any assays disclosed herein or known in the art. Examples of suitable animal models are described in more detail below.

Non-absorbed and/or effluxed phospholipase inhibitors of the present invention can form the basis of pharmaceutical compositions and kits that find use in methods of treating a subject by administering the composition. Preferably, such compositions modulate the activity of a gastrointestinal phospholipase, for example, reducing the activity of phospholipase A₂ and/or one or more other phospholipases. In some embodiments, the phospholipase inhibitor inhibits phospholipase A₂. In some embodiments, the phospholipase inhibitor inhibits phospholipase A₂ and phospholipase B. In some embodiments, the phospholipase inhibitor inhibits phospholipase A₂ but does not inhibit or does not significantly inhibit or essentially does not inhibit phospholipase B. In some embodiments, the phospholipase inhibitor inhibits phospholipase A₂ but does not inhibit or does not significantly inhibit or essentially does not inhibit other gastrointestinal phospholipases.

Methods of Treating Phospholipase-Related Conditions

The present invention provides methods of treating phospholipase-related conditions where the inhibitor is localized in a gastrointestinal lumen. Preferably, such inhibitors are administered orally, and preferably in a treatment protocol involving administering of PLA2 inhibitor during or shortly after meals.

The term “phospholipase-related condition” as used herein refers to a condition in which modulating the activity and/or re-absorption of a phospholipase, and/or modulating the production and/or effects of one or more products of the phospholipase, is desirable. In preferred embodiments, an inhibitor of the present invention reduces the activity and/or re-absorption of a phospholipase, and/or reduces the production and/or effects of one or more products of the phospholipase. The term “phospholipase A2-related condition” as used herein refers to a condition in which modulating the activity and/or re-absorption of phospholipase A2 is desirable and/or modulating the production and/or effects of one or more products of phospholipase A2 activity is desirable. In preferred embodiments, an inhibitor of the present invention reduces the activity and/or re-absorption of phospholipase A2, and/or reduces the production and/or effects of one or more products of the phospholipase A2. Examples of phospholipase A2-related conditions include, but are not limited to, insulin-related conditions (e.g., diabetes), weight-related conditions (e.g., obesity) and/or cholesterol-related conditions, and any combination thereof.

The present invention provides methods, pharmaceutical compositions, and kits for the treatment of animal subjects. The term “animal subject” as used herein includes humans as well as other mammals. For example, the mammals can be selected from mice, rats, rabbits, guinea pigs, hamsters, cats, dogs, porcine, poultry, bovine and horses, as well as combinations thereof.

The term “treating” as used herein includes achieving a therapeutic benefit and/or a prophylactic benefit. By therapeutic benefit is meant eradication or amelioration of the underlying disorder being treated. For example, in a diabetic patient, therapeutic benefit includes eradication or amelioration of the underlying diabetes. Also, a therapeutic benefit is achieved with the eradication or amelioration of one or more of the physiological symptoms associated with the underlying disorder such that an improvement is observed in the patient, notwithstanding the fact that the patient may still be afflicted with the underlying disorder. For example, with respect to diabetes reducing PL A₂ activity can provide therapeutic benefit not only when insulin resistance is corrected, but also when an improvement is observed in the patient with respect to other disorders that accompany diabetes like fatigue, blurred vision, or tingling sensations in the hands or feet. For prophylactic benefit, a phospholipase inhibitor of the present invention may be administered to a patient at risk of developing a phospholipase-related condition, e.g., diabetes, obesity, or hypercholesterolemia, or to a patient reporting one or more of the physiological symptoms of such conditions, even though a diagnosis may not have been made.

The present invention provides compositions comprising a phospholipase inhibitor that is not absorbed through a gastrointestinal mucosa and/or that is localized in a gastrointestinal lumen as a result of efflux from a gastrointestinal mucosal cell. In preferred embodiments, the phospholipase inhibitors of the present invention produce a benefit, including either a prophylactic benefit, a therapeutic benefit, or both, in treating one or more conditions by inhibiting phospholipase activity.

The methods for effectively inhibiting phospholipase described herein can apply to any phospholipase-related condition, that is, to any condition in which modulating the activity and/or re-absorption of a phospholipase, and/or modulating the production and/or effects of one or more products of the phospholipase, is desirable. Preferably, such conditions include phospholipase-A₂-related conditions and/or phospholipase A2-related conditions induced by diet, that is, conditions which are brought on, accelerated, exacerbated, or otherwise influenced by diet. Phospholipase-A₂-related conditions include, but are not limited to, diabetes, weight gain, and cholesterol-related conditions, as well as hyperlipidemia, hypercholesterolemia, cardiovascular disease (such as heart disease and stroke), hypertension, cancer, sleep apnea, osteoarthritis, gallbladder disease, fatty liver disease, diabetes type 2 and other insulin-related conditions. In some embodiments, one or more of these conditions may be produced as a result of consumption of a high fat or Western diet; in some embodiments, one or more of these conditions may be produced as a result of genetic causes, metabolic disorders, environmental factors, behavioral factors, or any combination of these.

Western Diets and Western-Related Diets

Generally, some embodiments of the invention relate to one or more of a high-carbohydrate diet, a high-saccharide diet, a high-fat diet and/or a high-cholesterol diet, in various combinations. Such diets are generally referred to herein as a “high-risk diets” (and can include for example, Western diets). Such diets can heighten the risk profile of a subject patient for one or more conditions, including an obesity-related condition, an insulin-related condition and/or a cholesterol-related condition. In particular, such high-risk diets can, in some embodiments, include at least a high-carbohydrate diet together with one or more of a high-saccharide diet, a high-fat diet and/or a high-cholesterol diet. A high-risk diet can also include a high-saccharide diet in combination with one or both of a high-fat diet and/or a high-cholesterol diet. A high-risk diet can also comprise a high-fat diet in combination with a high-cholesterol diet. In some embodiments, a high-risk diet can include the combination of a high-carbohydrate diet, a high-saccharide diet and a high-fat diet. In other embodiments, a high-risk diet can include a high-carbohydrate diet, a high-saccharide diet, and a high-cholesterol diet. In other embodiments, a high-risk diet can include a high-carbohydrate diet, a high-fat diet and a high-cholesterol diet. In yet further embodiments, a high-risk diet can include a high-saccharide diet, a high-fat diet and a high-cholesterol diet. In some embodiments, a high-risk diet can include a high-carbohydrate diet, a high-saccharide diet, a high-fat diet and a high-cholesterol diet.

Generally, the diet of a subject can comprise a total caloric content, for example, a total daily caloric content. In some embodiments, the subject diet can be a high-fat diet. In such embodiments, at least about 50% of the total caloric content can come from fat. In other such embodiments, at least about 40%, or at least about 30% or at least about 25%, or at least about 20% of the total caloric content can come from fat. In some embodiments, in which a high-fat diet is combined with one or more of a high-carbohydrate diet, a high-saccharide diet or a high-cholesterol diet, at least about 15% or at least about 10% of the total caloric content can come from fat.

Similarly, in some embodiments, the diet can be a high-carbohydrate diet. In such embodiments, at least about 50% of the total caloric content can come from carbohydrates. In other such embodiments, at least about 40%, or at least about 30% or at least about 25%, or at least about 20% of the total caloric content can come from carbohydrates. In some embodiments, in which a high-carbohydrate diet is combined with one or more of a high-fat diet, a high-saccharide diet or a high-cholesterol diet, at least about 15% or at least about 10% of the total caloric content can come from carbohydrate.

Further, in some embodiments, the diet can be a high-saccharide diet. In embodiments, at least about 50% of the total caloric content can come from saccharides. In other such embodiments, at least about 40%, or at least about 30% or at least about 25%, or at least about 20% of the total caloric content can come from saccharides. In some embodiments, in which a high-saccharide diet is combined with one or more of a high-fat diet, a high-carbohydrate diet or a high-cholesterol diet, at least about 15% or at least about 10% of the total caloric content can come from saccharides.

Similarly, in some embodiments, the diet can be a high-cholesterol diet. In such embodiments, the diet can comprise at least about 1% cholesterol (wt/wt, relative to fat). In other such embodiments, the diet can comprise at least about 0.5% or at least about 0.3% or at least about 0.1%, or at least about 0.07% cholesterol (wt/wt relative to fat). In some embodiments, in which a high-cholesterol diet is combined with one or more of a high-fat diet, a high-carbohydrate diet or a high-saccharide diet, the diet can comprise at least about 0.05% or at least about 0.03% cholesterol (wt/wt, relative to fat).

As an example, a high fat diet can include, for example, diets high in meat, dairy products, and alcohol, as well as possibly including processed food stuffs, red meats, soda, sweets, refined grains, deserts, and high-fat dairy products, for example, where at least about 25% of calories come from fat and at least about 8% come from saturated fat; or at least about 30% of calories come from fat and at least about 10% come from saturated fat; or where at least about 34% of calories came from fat and at least about 12% come from saturated fat; or where at least about 42% of calories come from fat and at least about 15% come from saturated fat; or where at least about 50% of calories come from fat and at least about 20% come from saturated fat. One such high fat diet is a “Western diet” which refers to the diet of industrialized countries, including, for example, a typical American diet, Western European diet, Australian diet, and/or Japanese diet. One particular example of a Western diet comprises at least about 17% fat and at least about 0.1% cholesterol (wt/wt); at least about 21% fat and at least about 0.15% cholesterol (wt/wt); or at least about 25% and at least about 0.2% cholesterol (wt/wt).

Such high-risk diets may include one or more high-risk foodstuffs.

Considered in the context of a foodstuff, generally, some embodiments of the invention relate to one or more of a high-carbohydrate foodstuff, a high-saccharide foodstuff, a high-fat foodstuff and/or a high-cholesterol foodstuff, in various combinations. Such foodstuffs are generally referred to herein as a “high-risk foodstuffs” (including for example Western foodstuffs). Such foodstuffs can heighten the risk profile of a subject patient for one or more conditions, including an obesity-related condition, an insulin-related condition and/or a cholesterol-related condition. In particular, such high-risk foodstuffs can, in some embodiments, include at least a high-carbohydrate foodstuff together with one or more of a high-saccharide foodstuff, a high-fat foodstuff and/or a high-cholesterol foodstuff. A high-risk foodstuff can also include a high-saccharide foodstuff in combination with one or both of a high-fat foodstuff and/or a high-cholesterol foodstuff. A high-risk foodstuff can also comprise a high-fat foodstuff in combination with a high-cholesterol foodstuff. In some embodiments, a high-risk foodstuff can include the combination of a high-carbohydrate foodstuff, a high-saccharide foodstuff and a high-fat foodstuff. In other embodiments, a high-risk foodstuff can include a high-carbohydrate foodstuff, a high-saccharide foodstuff, and a high-cholesterol foodstuff. In other embodiments, a high-risk foodstuff can include a high-carbohydrate foodstuff, a high-fat foodstuff and a high-cholesterol foodstuff. In yet further embodiments, a high-risk foodstuff can include a high-saccharide foodstuff, a high-fat foodstuff and a high-cholesterol foodstuff. In some embodiments, a high-risk foodstuff can include a high-carbohydrate foodstuff, a high-saccharide foodstuff, a high-fat foodstuff and a high-cholesterol foodstuff.

Hence, the food product composition can comprise a foodstuff having a total caloric content. In some embodiments, the food-stuff can be a high-fat foodstuff. In such embodiments, at least about 50% of the total caloric content can come from fat. In other such embodiments, at least about 40%, or at least about 30% or at least about 25%, or at least about 20% of the total caloric content can come from fat. In some embodiments, in which a high-fat foodstuff is combined with one or more of a high-carbohydrate foodstuff, a high-saccharide foodstuff or a high-cholesterol foodstuff, at least about 15% or at least about 10% of the total caloric content can come from fat.

Similarly, in some embodiments, the food-stuff can be a high-carbohydrate foodstuff. In such embodiments, at least about 50% of the total caloric content can come from carbohydrates. In other such embodiments, at least about 40%, or at least about 30% or at least about 25%, or at least about 20% of the total caloric content can come from carbohydrates. In some embodiments, in which a high-carbohydrate foodstuff is combined with one or more of a high-fat foodstuff, a high-saccharide foodstuff or a high-cholesterol foodstuff, at least about 15% or at least about 10% of the total caloric content can come from carbohydrate.

Further, in some embodiments, the food-stuff can be a high-saccharide foodstuff. In such embodiments, at least about 50% of the total caloric content can come from saccharides. In other such embodiments, at least about 40%, or at least about 30% or at least about 25%, or at least about 20% of the total caloric content can come from saccharides. In some embodiments, in which a high-saccharide foodstuff is combined with one or more of a high-fat foodstuff, a high-carbohydrate foodstuff or a high-cholesterol foodstuff, at least about 15% or at least about 10% of the total caloric content can come from saccharides.

Similarly, in some embodiments, the food-stuff can be a high-cholesterol foodstuff. In such embodiments, the food-stuff can comprise at least about 1% cholesterol (wt/wt, relative to fat). In other such embodiments, the foodstuff can comprise at least about 0.5%, or at least about 0.3% or at least about 0.1%, or at least about 0.07% cholesterol (wt/wt relative to fat). In some embodiments, in which a high-cholesterol foodstuff is combined with one or more of a high-fat foodstuff, a high-carbohydrate foodstuff or a high-saccharide foodstuff, the foodstuff can comprise at least about 0.05% or at least about 0.03% cholesterol (wt/wt, relative to fat).

As noted above, the methods of the invention can be used advantageously together with other methods, including for example methods broadly directed to treating insulin-related conditions, weight-related conditions and/or cholesterol-related conditions (including dislipidemia generally) and any combination thereof. Aspects of such conditions are described below.

Treatment of Insulin-Related Conditions

The term “insulin-related disorders” as used herein refers to a condition such as diabetes where the body does not produce and/or does not properly use insulin. Typically, a patient is diagnosed with pre-diabetes or diabetes by using a Fasting Plasma Glucose Test (FPG) and/or an Oral Glucose Tolerance Test (OGTT). In the case of the FPG test, a fasting blood glucose level between about 100 and about 125 mg/dl can indicate pre-diabetes; while a person with a fasting blood glucose level of about 126 mg/dl or higher can indicate diabetes. In the case of the OGTT test, a patient's blood glucose level can be measured after a fast and two hours after drinking a glucose-rich beverage. A two-hour blood glucose level between about 140 and about 199 mg/dl can indicate pre-diabetes; while a two-hour blood glucose level at about 200 mg/dl or higher can indicate diabetes.

In certain embodiments, a lumen localized phospholipase inhibitor of the present invention produces a benefit in treating an insulin-related condition, for example, diabetes, preferably diabetes type 2. For example, such benefits may include, but are not limited to, increasing insulin sensitivity and improving glucose tolerance. Other benefits may include decreasing fasting blood insulin levels, increasing tissue glucose levels and/or increasing insulin-stimulated glucose metabolism.

Without being limited to any particular hypothesis, these benefits may result from a number of effects brought about by reduced PL A₂ activity, including, for example, reduced membrane transport of phospholipids across the gastrointestinal mucosa and/or reduced production of 1-acyl lysophospholipids, such as 1-acyl lysophosphatydylcholine and/or reduced transport of lysophospholipids, 1-acyl lysophosphatydylcholine, that may act as a signaling molecule in subsequent pathways involved in diabetes or other insulin-related conditions.

In some embodiments, a lumen-localized phospholipase inhibitor is used that inhibits phospholipase A2 but does not inhibit or does not significantly inhibit or essentially does not inhibit phospholipase B. In some embodiments, the phospholipase inhibitor inhibits phospholipase A2 but no other gastrointestinal phospholipase, including not inhibiting or not significantly inhibiting or essentially not inhibiting phospholipase A1, and not inhibiting or not significantly inhibiting or essentially not inhibiting phospholipase.

Treatment of Weight-Related Conditions

The term “weight-related conditions” as used herein refers to unwanted weight gain, including overweight, obese and/or hyperlipidemic conditions, and in particular weight gain caused by a high fat or Western diet. Typically, body mass index (BMI) is used as the criteria in determining whether an individual is overweight and/or obese. An adult is considered overweight if, for example, he or she has a body mass index of at least about 25, and is considered obese with a BMI of at least about 30. For children, charts of Body-Mass-Index for Age are used, where a BMI greater than about the 85th percentile is considered “at risk of overweight” and a BMI greater than about the 95th percentile is considered “obese.”

In certain embodiments, a lumen localized phospholipase A2 inhibitor of the present invention can be used to treat weight-related conditions, including unwanted weight gain and/or obesity. In certain embodiments, a lumen localized phospholipase A2 inhibitor decreases fat absorption after a meal typical of a Western diet. In certain embodiments, a lumen localized phospholipase A2 inhibitor increases lipid excretion from a subject on a Western diet. In certain preferred embodiments, the phospholipase inhibitor reduces weight gain in a subject on a (typical) Western diet. In certain embodiments, practice of the present invention can preferentially reduce weight gain in certain tissues and organs, e.g., in some embodiments, a phospholipase A2 inhibitor can decrease weight gain in white fat of a subject on a Western diet.

Without being limited to any particular hypothesis, these benefits may result from a number of effects brought about by reduced PL A₂ activity. For example, inhibition of PL A₂ activity may reduce transport of phospholipids through the gastrointestinal lumen, for example, through the small intestine apical membrane, causing a depletion of the pool of phospholipids (e.g. phosphatidylcholine) in enterocytes, particularly in mammals fed with a high fat diet. In such cases, the de novo synthesis of phospholipids may not be sufficient to sustain the high turnover of phospholipids, e.g. phosphatidylcholine, needed to carry triglycerides, for example by transport in chylomicrons (See Tso, in Fat Absorption, 1986, chapt. 6 177-195, Kuksis A., Ed.), incorporated herein by reference.

PL A₂ inhibition can also reduce production of 1-acyl lysophospholipids, such as 1-acyl lysophosphatydylcholine, that may act as a signaling molecule in subsequent up-regulation pathways of fat absorption, including, for example the release of additional digestive enzymes or hormones, e.g., secretin. See, Huggins, Protection against diet-induced obesity and obesity-related insulin resistance in Group 1B-PL A₂-deficient mice, Am. J. Physiol. Endocrinol. Metab. 283:E994-E1001 (2002), incorporated herein by reference.

Another aspect of the present invention provides composition, kits and methods for reducing or delaying the onset of diet-induced diabetes through weight gain. An unchecked high fat diet can produce not only weight gain, but also can contribute to diabetic insulin resistance. This resistance may be recognized by decreased insulin and leptin levels in a subject. The phospholipase inhibitors, compositions, kits and methods disclosed herein can be used in the prophylactic treatment of diet-induced diabetes, or other insulin-related conditions, e.g. in decreasing insulin and/or leptin levels in a subject on a Western diet.

In some embodiments, a lumen-localized phospholipase inhibitor is used that inhibits phospholipase A2 but does not inhibitor or does not significantly inhibit or essentially does not inhibit phospholipase B. In some embodiments, the phospholipase inhibitor inhibits phospholipase A2 but no other gastrointestinal phospholipase, including not inhibiting or not significantly inhibiting or essentially not inhibiting phospholipase A1, and not inhibiting or not significantly inhibiting or essentially not inhibiting phospholipase B.

Treatment of Cholesterol-Related Conditions

The term “cholesterol-related conditions” as used herein refers to a condition in which modulating the activity of HMG-CoA reductase is desirable and/or modulating the production and/or effects of one or more products of HMG-CoA reductase is desirable. In preferred embodiments, a phospholipase inhibitor of the present invention reduces the activity of HMG-CoA reductase and/or reduces the production and/or effects of one or more products of HMG-CoA reductase. For example, a cholesterol-related condition may involve elevated levels of cholesterol, in particular, non-HDL cholesterol in plasma (e.g., elevated levels of LDL cholesterol and/or VLDL/LDL levels). Typically, a patient is considered to have high or elevated cholesterol levels based on a number of criteria, for example, see Pearlman B L, The New Cholesterol Guidelines, Postgrad Med, 2002; 112(2):13-26, incorporated herein by reference. Guidelines include serum lipid profiles, such as LDL compared with HDL levels.

Examples of cholesterol-related conditions include hypercholesterolemia, lipid disorders such as hyperlipidemia, and atherogenesis and its sequelae of cardiovascular diseases, including atherosclerosis, other vascular inflammatory conditions, myocardial infarction, ischemic stroke, occlusive stroke, and peripheral vascular diseases, as well as other conditions in which decreasing cholesterol can produce a benefit. Other cholesterol-related conditions treatable with compositions, kits, and methods of the present invention include those currently treated with statins, as well as other conditions in which decreasing cholesterol absorption can produce a benefit.

In certain embodiments, a lumen-localized phospholipase inhibitor of the present invention can be used to reduce cholesterol levels, in particular non-HDL plasma cholesterol levels, e.g. by reducing cholesterol absorption. In some preferred embodiments, the composition inhibits phospholipase A2 and at least one other gastrointestinal phospholipase in addition to phospholipase A2, such as preferably phospholipase B, and also such as phospholipase A1, phospholipase C, and/or phospholipase D.

In other embodiments of the invention, the differential activities of phospholipases can be used to treat certain phospholipase-related conditions without undesired side effects resulting from inhibiting other phospholipases. For example, in certain embodiments, a phospholipase inhibitor that inhibits PL A₂, but not inhibiting or not significantly inhibiting or essentially not inhibiting, for example, PLA1, PLB, PLC, or PLD can be used to treat an insulin-related condition (e.g. diabetes) and/or a weight-related condition (e.g. obesity) without affecting, or without significantly affecting, or without essentially effecting, cholesterol absorption of a subject receiving phospholipase inhibiting treatment, e.g., when the subject is on a high fat diet.

Other cholesterol-related conditions of particular interest include dislipidemia conditions, such as hypertriglyceridemia. Hepatic triglyceride synthesis is regulated by available fatty acids, glycogen stores, and the insulin versus glucagon ratio. Patients with a high glucose diet (including, for example, patients on a high-carbohydrate or a high-saccharide diet, and/or patients in a population known to typically consume such diets) are likely to have a balance of hormones that maintains an excess of insulin and also build up glycogen stores, both of which enhance hepatic triglyceride synthesis. In addition, diabetic patients are particularly susceptible, since they are often overweight and are in a state of caloric excess. Hence, the present invention is particularly of interest, in each embodiment herein described, with respect to treatments directed to hypertriglyceridemia.

Without being bound by theory not specifically recited in the claims, the phospholipase A2 inhibitors of the present invention can modulate triglycerides and cholesterol through more than one mechanistic path. For example, the phospholipase A2 inhibitors of the invention can modulate cholesterol absorption and triglyceride absorption from the gastrointestinal tract, and can also modulate the metabolism of fat and glucose, for example, via signaling molecules such as lysophosphatidylcholine (the reaction product of PLA2 catalyzed hydrolysis of phosphatidylcholine), operating directly and/or in conjunction with other hormones such as insulin. Such metabolic modulation can directly impact serum cholesterol and triglyceride levels in patients on a high fat/high disaccharide diet or on a high fat/high carbohydrate diet. VLDL is a lipoprotein packaged by the liver for endogenous circulation from the liver to the peripheral tissues. VLDL contains triglycerides, cholesterol, and phospholipase at its core along with apolipoproteins B100, C1, CII, CIII, and E at its perimeter. Triglycerides make up more than half of VLDL by weight and the size of VLDL is determined by the amount of triglyceride. Very large VLDL is secreted by the liver in states of caloric excess, in diabetes mellitus, and after alcohol consumption, because excess triglycerides are present. As such, inhibition of phospholipase A2 activity can impact metabolism, including for example hepatic triglyceride synthesis. Modulated (e.g., reduced or at least relatively reduced increase) in triglyceride synthesis can provide a basis for modulating serum triglyceride levels and/or serum cholesterol levels, and further can provide a basis for treating hypertriglyceridemia and/or hypercholesterolemia. Such treatments would be beneficial to both diabetic patients (who typically replace their carbohydrate restrictions with higher fat meals), and to hypertriglyceridemic patients (who typically substitute fat with high carbohydrate meals). In this regard, increased protein meals alone are usually not sustainable in the long term for most diabetic and/or hypertriglyceridemic patients.

Moreover, the modulation of serum triglyceride levels can have a beneficial effect on cardiovascular diseases such as atherosclerosis. Triglycerides included in VLDL packaged and released from the liver into circulation are in turn, hydrolyzed by lipoprotein lipase, such that VLDL are converted to VLDL remnants (=IDL). VLDL remnants can either enter the liver (the large ones preferentially do this) or can give rise to LDL. Hence, elevated VLDL in the circulation lowers HDL, which is responsible for reverse cholesterol transport. Since hypertriglyceridemia contributes to elevated LDL levels and also contributes to lowered HDL levels, hypertriglyceridemia is a risk factor for cardiovascular diseases such as atherosclerosis and coronary artery disease (among others, as noted above). Accordingly, modulating hypertriglyceridemia using the phospholipase-A2 inhibitors of the present invention also provide a basis for treating such cardiovascular diseases.

The phospholipase inhibitors, methods, and kits disclosed herein can be used in the treatment of phospholipase-related conditions. In some preferred embodiments, these effects can be realized without a change in diet and/or activity on the part of the subject. For example, the activity of PL A₂ in the gastrointestinal lumen may be inhibited to result in a decrease in fat absorption and/or a reduction in weight gain in a subject on a Western diet compared to if the subject was not receiving PL A₂ inhibiting treatment. More preferably, this decrease and/or reduction occurs without a change, without a significant change, or essentially without a change, in energy expenditure and/or food intake on the part of the subject, and without a change, or without a significant change, or essentially without a change in the body temperature of the subject. Further, in preferred embodiments, a phospholipase inhibitor of the present invention can be used to offset certain negative consequences of high fat diets without affecting normal aspects of metabolism on non-high fat diets.

The present invention also includes kits that can be used to treat phospholipase-related conditions, preferably phospholipase A2-related conditions or phospholipase-related conditions induced by diet, including, but not limited to, insulin-related conditions (e.g., diabetes, particularly diabetes type 2), weight-related conditions (e.g., obesity) and/or cholesterol-related conditions. These kits comprise at least one composition of the present invention and instructions teaching the use of the kit according to the various methods described herein.

Treatments Using Inhibitors Comprising Fused Five-and-Six-Membered Rings

In some preferred embodiments, phospholipase-related conditions can be treated (especially diet-related conditions prevalent in populations consuming high-fat diets, and therefore being at risk of diet-induced conditions such as obesity, diabetes, insulin resistance, and glucose intolerance) using lumen-localized inhibitors comprising a small organic molecule phospholipase inhibitor or inhibiting moiety that comprises or is derived from a substituted organic compound having a fused five-member ring and six-member ring, and preferably a fused five-member ring and six-member ring having one or more heteroatoms (e.g., nitrogen, oxygen) substituted within the ring structure of the five-member ring, within the ring structure of the six-member ring, or within the ring structure of each of the five-member and six-member rings. In each case the inhibiting moiety can comprise substituent groups effective for imparting phospholipase inhibiting functionality to the moiety. The inhibiting moiety can also include a substituent having functionality for linking directly or indirectly to the polymer moiety. In especially preferred embodiments, phospholipase-related conditions can be treated using a phospholipase inhibitor or inhibiting moiety that comprises an indole moiety, such as a substituted indole moiety. Such small molecule inhibitors or inhibiting moieties have been found to be especially effective in treating phospholipase-related conditions. (See, for example, PCT Appl. No. US/2005/______ entitled “Treatment of Diet-Related Conditions Using Phospholipase-A2 Inhibitors Comprising Indoles and Related Compounds” filed on May 3, 2005 by Buysse et al.; See also PCT Appl. No. US/2005/______ entitled “Treatment Hypercholesterolemia, Hypertriglyceridemia and Cardiovascular-Related Conditions Using Phospholipase-A2 Inhibitors” filed on May 3, 2005 by Charmot et al., each of which is incorporated herein by reference).

Inhibitor Formulations, Routes of Administration, and Effective Doses

The phospholipase inhibitors useful in the present invention, or pharmaceutically acceptable salts thereof, can be delivered to a patient using a number of routes or modes of administration. The term “pharmaceutically acceptable salt” means those salts which retain the biological effectiveness and properties of the compounds used in the present invention, and which are not biologically or otherwise undesirable. Such salts include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, if the compounds used in the present invention contain a carboxyl group or other acidic group, it may be converted into a pharmaceutically acceptable addition salt with inorganic or organic bases. Examples of suitable bases include sodium hydroxide, potassium hydroxide, ammonia, cyclohexylamine, dicyclohexyl-amine, ethanolamine, diethanolamine and triethanolamine.

If necessary or desirable, the phospholipase inhibitor may be administered in combination with one or more other therapeutic agents. The choice of therapeutic agent that can be co-administered with a composition of the invention will depend, in part, on the condition being treated. For example, for treating obesity, or other weight-related conditions, a phospholipase inhibitor of some embodiments of the present invention can be used in combination with a statin, a fibrate, a bile acid binder, an ezitimibe (e.g., Zetia, etc), a saponin, a lipase inhibitor (e.g. Orlistat, etc), and/or an appetite suppressant, and the like. With respect to treating insulin-related conditions, e.g., diabetes, a phospholipase inhibitor of some embodiments the present invention can be used in combination with a biguanide (e.g., Metformin), thiazolidinedione, and/or α-glucosidase inhibitor, and the like.

The phospholipase inhibitors (or pharmaceutically acceptable salts thereof) may be administered per se or in the form of a pharmaceutical composition wherein the active compound(s) is in admixture or mixture with one or more pharmaceutically acceptable carriers, excipients or diluents. Pharmaceutical compositions for use in accordance with the present invention may be formulated in conventional manner using one or more physiologically acceptable carriers compromising excipients and auxiliaries which facilitate processing of the active compounds into preparations which can be used pharmaceutically. Proper formulation is dependent upon the route of administration chosen.

The phospholipase inhibitors can be administered by direct placement, orally, and/or rectally. Preferably, the phospholipase inhibitor or the pharmaceutical composition comprising the phospholipase inhibitor is administered orally. The oral form in which the phospholipase inhibitor is administered can include a powder, tablet, capsule, solution, or emulsion. The effective amount can be administered in a single dose or in a series of doses separated by appropriate time intervals, such as hours.

For oral administration, the compounds can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions, wafers, and the like, for oral ingestion by a patient to be treated. In some embodiments, the inhibitor may be formulated as a sustained release preparation. Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinyl pyrrolidone (PVP). If desired, disintegrating agents may be added, such as the cross-linked polyvinyl pyrrolidone, agar, or alginic acid or a salt thereof such as sodium alginate.

Dragee cores can be provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses. In some embodiments, the oral formulation does not have an enteric coating.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for administration.

Suitable carriers used in formulating liquid dosage forms for oral as well as parenteral administration include non-aqueous, pharmaceutically-acceptable polar solvents such as hydrocarbons, alcohols, amides, oils, esters, ethers, ketones, and/or mixtures thereof, as well as water, saline solutions, electrolyte solutions, dextrose solutions (e.g., DW5), and/or any other aqueous, pharmaceutically acceptable liquid.

Suitable nonaqueous, pharmaceutically-acceptable polar solvents include, but are not limited to, alcohols (e.g., aliphatic or aromatic alcohols having 2-30 carbon atoms such as methanol, ethanol, propanol, isopropanol, butanol, t-butanol, hexanol, octanol, benzyl alcohol, amylene hydrate, glycerin (glycerol), glycol, hexylene glycol, lauryl alcohol, cetyl alcohol, stearyl alcohol, tetrahydrofurfuryl alcohol, fatty acid esters of fatty alcohols such as polyalkylene glycols (e.g., polyethylene glycol and/or polypropylene glycol), sorbitan, cholesterol, sucrose and the like); amides (e.g., dimethylacetamide (DMA), benzyl benzoate DMA, N,N-dimethylacetamide amides, 2-pyrrolidinone, polyvinylpyrrolidone, 1-methyl-2-pyrrolidinone, and the like); esters (e.g., 2-pyrrolidinone, 1-methyl-2-pyrrolidinone, acetate esters (such as monoacetin, diacetin, and triacetin and the like), and the like, aliphatic or aromatic esters (such as dimethylsulfoxide (DMSO), alkyl oleate, ethyl caprylate, ethyl benzoate, ethyl acetate, octanoate, benzyl benzoate, benzyl acetate, esters of glycerin such as mono, di, or tri-glyceryl citrates or tartrates, ethyl carbonate, ethyl oleate, ethyl lactate, N-methyl pyrrolidinone, fatty acid esters such as isopropyl myristrate, fatty acid esters of sorbitan, glyceryl monostearate, glyceride esters such as mono, di, or tri-glycerides, fatty acid derived PEG esters such as PEG-hydroxystearate, PEG-hydroxyoleate, and the like, pluronic 60, polyoxyethylene sorbitol oleic polyesters, polyoxyethylene sorbitan esters such as polyoxyethylene-sorbitan monooleate, polyoxyethylene-sorbitan monostearate, polyoxyethylene-sorbitan monolaurate, polyoxyethylene-sorbitan monopalmitate, alkyleneoxy modified fatty acid esters such as polyoxyl 40 hydrogenated castor oil and polyoxyethylated castor oils, saccharide fatty acid esters (i.e., the condensation product of a monosaccharide, disaccharide, or oligosaccharide or mixture thereof with a fatty acid(s) (e.g., saturated fatty acids such as caprylic acid, myristic acid, palmitic acid, capric acid, lauric acid, and stearic acid, and unsaturated fatty acids such as palmitoleic acid, oleic acid, elaidic acid, erucic acid and linoleic acid)), or steroidal esters and the like); alkyl, aryl, or cyclic ethers (e.g., diethyl ether, tetrahydrofuran, diethylene glycol monoethyl ether, dimethyl isosorbide and the like); glycofurol (tetrahydrofurfuryl alcohol polyethylene glycol ether); ketones (e.g., acetone, methyl isobutyl ketone, methyl ethyl ketone and the like); aliphatic, cycloaliphatic or aromatic hydrocarbons (e.g., benzene, cyclohexane, dichloromethane, dioxolanes, hexane, n-hexane, n-decane, n-dodecane, sulfolane, tetramethylenesulfoxide, tetramethylenesulfon, toluene, tetramethylenesulfoxide dimethylsulfoxide (DMSO) and the like); oils of mineral, animal, vegetable, essential or synthetic origin (e.g., mineral oils such as refined paraffin oil, aliphatic or wax-based hydrocarbons, aromatic hydrocarbons, mixed aliphatic and aromatic based hydrocarbons, and the like, vegetable oils such as linseed, soybean, castor, rapeseed, coconut, tung, safflower, cottonseed, groundnut, palm, olive, corn, corn germ, sesame, persic, peanut oil, and the like, as well as glycerides such as mono-, di- or triglycerides, animal oils such as cod-liver, haliver, fish, marine, sperm, squalene, squalane, polyoxyethylated castor oil, shark liver oil, oleic oils, and the like); alkyl or aryl halides e.g., methylene chloride; monoethanolamine; trolamine; petroleum benzin; omega-3 polyunsaturated fatty acids (e.g., α-linolenic acid, docosapentaenoic acid, docosahexaenoic acid, eicosapentaenoic acid, and the like); polyglycol ester of 12-hydroxystearic acid; polyethylene glycol; polyoxyethylene glycerol, and the like.

Other pharmaceutically acceptable solvents that can be used in formulating pharmaceutical compositions of a phospholipase inhibitor of the present invention including, for example, for direct placement, are well known to those of ordinary skill in the art, e.g. see Modern Pharmaceutics, (G. Banker et al., eds., 3d ed.)(Marcel Dekker, Inc., New York, N.Y., 1995), The Handbook of Pharmaceutical Excipients, (American Pharmaceutical Association, Washington, D.C.; The Pharmacological Basis of Therapeutics, (Goodman & Gilman, McGraw Hill Publishing), Remington's Pharmaceutical Sciences (A. Gennaro, ed., 19th ed.) (Mack Publishing, Easton, Pa., 1995), Pharmaceutical Dosage Forms, (H. Lieberman et al., eds.,) (Marcel Dekker, Inc., New York, N.Y., 1980); and The United States Pharmacopeia 24, The National Formulary 19, (National Publishing, Philadelphia, Pa., 2000).

Formulations for rectal administration may be prepared in the form of a suppository, an ointment, an enema, a tablet, or a cream for release of the phospholipase inhibitor in the gastrointestinal tract, e.g., the small intestine. Rectal suppositories can be made by mixing one or more phospholipase inhibitors of the present invention, or pharmaceutically acceptable salts thereof, with acceptable vehicles, for example, cocoa butter, with or without the addition of waxes to alter melting point. Acceptable vehicles can also include glycerin, salicylate and/or polyethylene glycol, which is solid at normal storage temperature, and a liquid at those temperatures suitable to release the phospholipase inhibitor inside the body, such as in the rectum. Oils may also be used in rectal formulations of the soft gelatin type and in suppositories. Water soluble suppository bases, such as polyethylene glycols of various molecular weights, may also be used. Suspension formulations may be prepared that use water, saline, aqueous dextrose and related sugar solutions, and glycerols, as well as suspending agents such as pectins, carbomers, methyl cellulose, hydroxypropyl cellulose or carboxymethyl cellulose, as well as buffers and preservatives.

Pharmaceutical compositions suitable for use in the present invention include compositions wherein the active ingredients are present in an effective amount, i.e., in an amount sufficient to produce a therapeutic and/or a prophylactic benefit in at least one condition being treated. The actual amount effective for a particular application will depend on the condition being treated and the route of administration. Determination of an effective amount is well within the capabilities of those skilled in the art, especially in light of the disclosure herein. For example, the IC50 values and ranges provided in Table 1 above provide guidance to enable one of ordinary skill in the art to select effective dosages of the corresponding phospholipase inhibiting moieties.

The effective amount when referring to a phospholipase inhibitor will generally mean the dose ranges, modes of administration, formulations, etc., that have been recommended or approved by any of the various regulatory or advisory organizations in the medical or pharmaceutical arts (eg, FDA, AMA) or by the manufacturer or supplier. Effective amounts of phospholipase inhibitors can be found, for example, in the Physicians Desk Reference. The effective amount when referring to producing a benefit in treating a phospholipase-related condition, such as insulin-related conditions (e.g., diabetes), weight-related conditions (e.g., obesity), and/or cholesterol related-conditions will generally mean the levels that achieve clinical results recommended or approved by any of the various regulatory or advisory organizations in the medical or pharmaceutical arts (eg, FDA, AMA) or by the manufacturer or supplier.

A person of ordinary skill using techniques known in the art can determine the effective amount of the phospholipase inhibitor. In the present invention, the effective amount of a phospholipase inhibitor localized in the gastrointestinal lumen can be less than the amount administered in the absence of such localization. Even a small decrease in the amount of phospholipase inhibitor administered is considered useful for the present invention. A significant decrease or a statistically significant decrease in the effective amount of the phospholipase inhibitor is particularly preferred. In some embodiments of the invention, the phospholipase inhibitor reduces activity of phospholipase to a greater extent compared to non-lumen localized inhibitors. Lumen-localization of the phospholipase inhibitor can decrease the effective amount necessary for the treatment of phospholipase-related conditions, such as insulin-related conditions (e.g., diabetes), weight-related conditions (e.g., obesity) and/or cholesterol-related conditions by about 5% to about 95%. The amount of phospholipase inhibitor used could be the same as the recommended dosage or higher than this dose or lower than the recommended dose.

In some embodiments, the recommended dosage of a phospholipase inhibitor is between about 0.1 mg/kg/day and about 1,000 mg/kg/day. The effective amount for use in humans can be determined from animal models. For example, a dose for humans can be formulated to achieve circulating and/or gastrointestinal concentrations that have been found to be effective in animals, e.g. a mouse model as the ones described in the samples below.

A person of ordinary skill in the art can determine phospholipase inhibition by measuring the amount of a product of a phospholipase, e.g., lysophosphatidylcholine (LPC), a product of PL A₂. The amount of LPC can be determined, for example, by measuring small intestine, lymphatic, and/or serum levels post-prandially. Another technique for determining amount of phospholipase inhibition involves taking direct fluid samples from the gastrointestinal tract. A person of ordinary skill in the art would also be able to monitor in a patient the effect of a phospholipase inhibitor of the present invention, e.g., by monitoring cholesterol and/or triglyceride serum levels. Other techniques would be apparent to one of ordinary skill in the art. Other approaches for measuring phospholipase inhibition and/or for demonstrating the effects of phospholipase inhibitors of some embodiments are further illustrated in the examples below.

### EXAMPLES

### Example 1A

### Synthesis of ILY-4001 [2-(3-(2-amino-2-oxoacetyl)-1-(biphenyl-2-ylmethyl)-2-methyl-1H-indol-4-yloxy)acetic acid]

This example synthesized a compound for use as a phospholipase inhibitor or inhibiting moiety. Specifically, the compound 2-(3-(2-amino-2-oxoacetyl)-1-(biphenyl-2-ylmethyl)-2-methyl-1H-indol-4-yloxy)acetic acid, shown in FIG. 2 was synthesized. This compound is designated in these examples as ILY-4001, and is alternatively referred to herein as methyl indoxam.

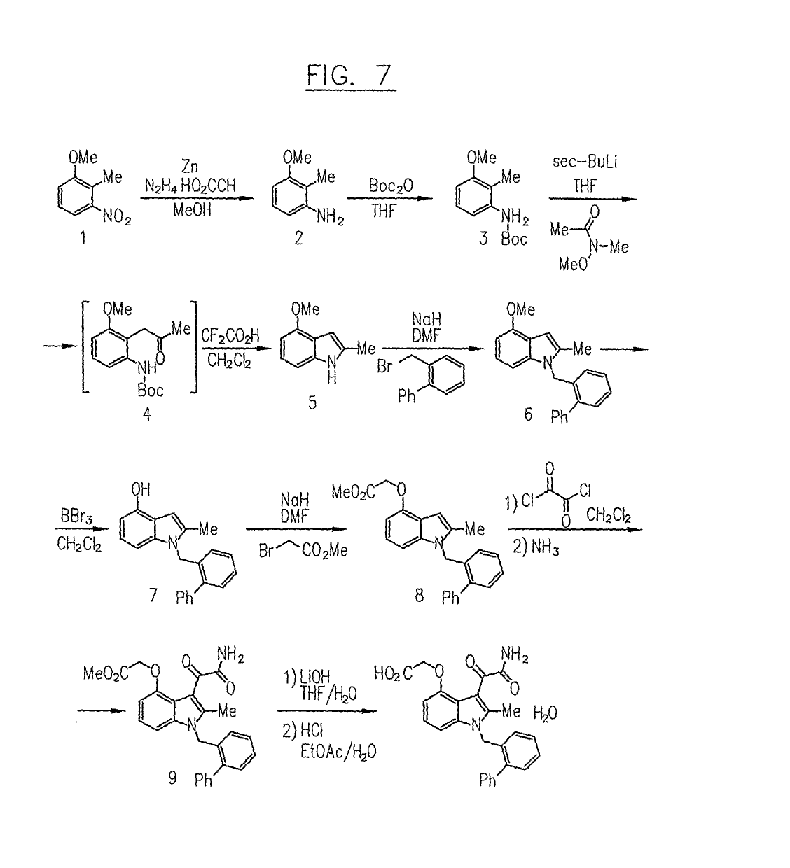

Reference is made to FIG. 7, which outlines the overall synthesis scheme for ILY-4001. The numbers under each compound shown in FIG. 7 correspond to the numbers in parenthesis associated with the chemical name for each compound in the following experimental description.

2-Methyl-3-methoxyaniline (2) [04-035-11]. To a stirred cooled (ca. 5° C.) hydrazine hydrate (159.7 g, 3.19 mol), 85% formic acid (172.8 g, 3.19 mol) was added drop wise at 10-20° C. The resultant mixture was added drop wise to a stirred suspension of zinc dust (104.3 g, 1.595 mol) in a solution of 2-methyl-3-nitroanisole (1) (53.34 g, 0.319 mol) in methanol (1000 mL). An exothermic reaction occurred. After the addition was complete, the reaction mixture was stirred for additional 2 h (until temperature dropped from 61° C. to RT) and the precipitate was filtered off and washed with methanol (3×150 mL). The filtrate was concentrated under reduced pressure to a volume of ca. 250 mL. The residue was treated with EtOAc (500 ml) and saturated aqueous NaHCO₃ (500 mL). The aqueous phase was separated off and discarded. The organic phase was washed with water (300 mL) and extracted with 1N HCl (800 mL). The acidic extract was washed with EtOAc (300 mL) and was basisified with K₂CO₃ (90 g). The free base 2 was extracted with EtOAc (3×200 mL) and the combined extracts were dried over MgSO₄. After filtration and removal of the solvent from the filtrate, product 2 was obtained as a red oil, which was used in the next step without further purification. Yield: 42.0 g (96%).

N-tert-Butyloxycarbonyl-2-methyl-3-methoxyaniline (3) [04-035-12]. A stirred solution of amine 2 (42.58 g, 0.31 mol) and di-tert-butyl dicarbonate (65.48 g, 0.30 mol) in THF (300 mL) was heated to maintain reflux for 4 h. After cooling to RT, the reaction mixture was concentrated under reduced pressure and the residue was dissolved in EtOAc (500 mL). The resultant solution was washed with 0.5 M citric acid (2×100 mL), water (100 mL), saturated aqueous NaHCO₃ (200 mL), brine (200 mL) and dried over MgSO₄. After filtration and removal of the solvent from the filtrate, the residue (red oil, 73.6 g) was dissolved in hexanes (500 mL) and filtered through a pad of Silica Gel (for TLC). The filtrate was evaporated under reduced pressure to provide N—Boc aniline 3 as a yellow solid. Yield: 68.1 g (96%).

4-Methoxy-2-methyl-1H-indole (5) [04-035-13]. To a stirred cooled (−50° C.) solution of N-Boc aniline 3 (58.14 g, 0.245 mol) in anhydrous THF (400 mL), a 1.4 M solution of sec-BuLi in cyclohexane (0.491 mol, 350.7 mL) was added drop wise at −48-−50° C. and the reaction mixture was allowed to warm up to −20° C. After cooling to −60° C., a solution of N-methoxy-N-methylacetamide (25.30 g, 0.245 mol) in THF (25 mL) was added drop wise at −57-−60° C. The reaction mixture was stirred for 1 h at −60° C. and was allowed to warm up to 15° C. during 1 h. After cooling to −15° C., the reaction was quenched with 2N HCl (245 mL) and the resultant mixture was adjusted to pH of ca. 7 with 2N HCl. The organic phase was separated off and saved. The aqueous phase was extracted with EtOAc (3×100 mL). The organic solution was concentrated under reduced pressure and the residual pale oil was dissolved in EtOAc (300 mL) and combined with the EtOAc extracts. The resultant solution was washed with water (2×200 mL), 0.5 M citric acid, (100 mL), saturated aqueous NaHCO₃ (100 mL), brine (200 mL) and dried over MgSO₄. After filtration and removal of the solvent from the filtrate, a mixture of starting N-Boc aniline 3 and intermediate ketone 4 (ca. 1:1 mol/mol) was obtained as a pale oil (67.05 g).

The obtained oil was dissolved in anhydrous CH₂Cl₂ (150 mL) and the solution was cooled to 0-−5° C. Trifluoroacetic acid (65 mL) was added drop wise and the reaction mixture was allowed to warm up to RT. After 16 h of stirring, an additional portion of trifluoroacetic acid (35 mL) was added and stirring was continued for 16 h. The reaction mixture was concentrated under reduced pressure and the red oily residue was dissolved in CH₂Cl₂ (500 mL). The resultant solution was washed with water (3×200 mL) and dried over MgSO₄. Filtration through a pad of Silica Gel 60 and evaporation of the filtrate under reduced pressure provided crude product 5 as a yellow solid (27.2 g). Purification by dry chromatography (Silica Gel for TLC, 20% EtOAc in hexanes) afforded indole 5 as a white solid. Yield: 21.1 g (53%)

1-[(1,1′-Biphenyl)-2-ylmethyl]-4-methoxy-2-methyl-1H-indole (6) [04-035-14]. A solution of indole 5 (16.12 g, 0.10 mol) in anhydrous DMF (100 mL) was added drop wise to a stirred cooled (ca. 15° C.) suspension of sodium hydride (0.15 mol, 6.0 g, 60% in mineral oil, washed with 100 mL of hexanes before the reaction) in DMF (50 mL) and the reaction mixture was stirred for 0.5 h at RT. After cooling the reaction mixture to ca. 5° C., 2-phenylbenzyl bromide (25.0 g, 0.101 mol) was added drop wise and the reaction mixture was stirred for 18 h at RT. The reaction was quenched with water (10 mL) and EtOAc (500 mL) was added. The resultant mixture was washed with water (2×200 mL+3×100 mL), brine (200 mL) and dried over MgSO₄. After filtration and removal of the solvent from the filtrate under reduced pressure, the residue (35.5 g, thick red oil) was purified by dry chromatography (Silica Gel for TLC, 5%→25% CH₂Cl₂ in hexanes) to afford product 6 as a pale oil. Yield: 23.71 g (72%).

1-[(1,1′-Biphenyl)-2-ylmethyl]-4-hydroxy-2-methyl-1H-indole (7) [04-035-15]. To a stirred cooled (ca. 10° C.) solution of the methoxy derivative 6 (23.61 g, 72.1 mmol) in anhydrous CH₂Cl₂ (250 mL), a 1M solution of BBr₃ in CH₂Cl₂ (300 mmol, 300 mL) was added drop wise at 15-20° C. and the dark reaction mixture was stirred for 5 h at RT. After concentrating of the reaction mixture under reduced pressure, the dark oily residue was cooled to ca. 5° C. and was dissolved in precooled (15° C.) EtOAc (450 mL). The resultant cool solution was washed with water (3×200 mL), brine (200 mL) and dried over MgSO₄. After filtration and removal of the solvent from the filtrate under reduced pressure, the residue (26.1 g, dark semi-solid) was purified by dry chromatography (Silica Gel for TLC, 5%→25% EtOAc in hexanes) to afford product 7 as a brown solid. Yield: 4.30 g (19%)

2-{1-[(1,1′-Biphenyl)-2-ylmethyl)-2-methyl-1H-indol-4-yl]oxy}-acetic acid methyl ester (8) [04-035-16]. To a stirred suspension of sodium hydride (0.549 g, 13.7 mmol, 60% in mineral oil) in anhydrous DMF (15 mL), a solution of compound 7 (4.30 g, 13.7 mmol) in DMF (30 mL) was added drop wise and the resultant mixture was stirred for 40 min at RT. Methyl bromoacetate (2.10 g, 13.7 mmol) was added drop wise and stirring was continued for 21 h at RT. The reaction mixture was diluted with EtOAc (200 mL) and washed with water (4×200 mL), brine (200 mL) and dried over MgSO₄. After filtration and removal of the solvent from the filtrate under reduced pressure, the residue (5.37 g, dark semi-solid) was purified by dry chromatography (Silica Gel for TLC, 5%→30% EtOAc in hexanes) to afford product 8 as a yellow solid. Yield: 4.71 g (89%).

2-{[3-(2-Amino-1,2-dioxoethyl)-1-[(1,1′-biphenyl)-2-ylmethyl)-2-methyl-1H-indol-4-yl]oxy}-acetic acid methyl ester (9) [04-035-17]. To a stirred solution of oxalyl chloride (1.55 g, 12.2 mmol) in anhydrous CH₂Cl₂ (20 mL), a solution of compound 8 in CH₂Cl₂ (40 mL) was added drop wise and the reaction mixture was stirred for 80 min at RT. After cooling the reaction mixture to −10° C., a saturated solution of NH₃ in CH₂Cl₂ (10 mL) was added drop wise and then the reaction mixture was saturated with NH₃ (gas) at ca. 0° C. Formation of a precipitate was observed. The reaction mixture was allowed to warm up to RT and was concentrated under reduced pressure to dryness. The dark solid residue (6.50 g) was subjected to dry chromatography (Silica Gel for TLC, 30% EtOAc in hexanes→100% EtOAc) to afford product 9 as a yellow solid. Yield: 4.64 g (83%).

2-{[3-(2-Amino-1,2-dioxoethyl)-1-[(1,1′-biphenyl)-2-ylmethyl)-2-methyl-1H-indol-4-yl]oxy}-acetic acid (ILY-4001) [04-035-18]. To a stirred solution of compound 9 (4.61 g, 10.1 mmol) in a mixture of THF (50 mL) and water (10 mL), a solution of lithium hydroxide monohydrate (0.848 g, 20.2 mmol) in water (20 mL) was added portion wise and the reaction mixture was stirred for 2 h at RT. After addition of water (70 mL), the reaction mixture was concentrated under reduced pressure to a volume of ca. 100 mL. Formation of a yellow precipitate was observed. To the residual yellow slurry, 2N HCl (20 mL) and EtOAc (200 mL) were added and the resultant mixture was stirred for 16 h at RT. The yellowish-greenish precipitate was filtered off and washed with EtOAc (3×20 mL), Et₂O (20 mL) and hexanes (20 mL). After drying in vacuum, the product (2.75 g) was obtained as a pale solid. MS: 443.27 (M⁺+1). Elemental Analysis Calcd for C₂₆H₂₂N₂O₅+H₂O: C, 67.82; H, 5.25; N, 6.08. Found: C, 68.50; H, 4.96; N, 6.01. HPLC: 96.5% purity. ¹H NMR (DMSO-d₆) δ 7.80 (br s, 1H), 7.72-7.25 (m, 9H), 7.07 (t, 1H), 6.93 (d, 1H), 6.57 (d, 1H), 6.43 (d, 1H), 5.39 (s, 2H), 4.68 (s, 2H), 2.38 (s, 3H).

The aqueous phase of the filtrate was separated off and the organic one was washed with brine (100 mL) and dried over MgSO₄. After filtration and removal of the solvent from the filtrate under reduced pressure, the greenish solid residue was washed with EtOAc (3×10 mL), Et₂O (10 mL) and hexanes (10 mL). After drying in vacuum, an additional portion (1.13 g) of product was obtained as a greenish solid. Total yield: 2.75 g+1.13 g=3.88 g (87%).

### Example 1B

### Characterization Studies

### ILY-4001 [2-(3-(2-amino-2-oxoacetyl)-4-(biphenyl-2-ylmethyl)-2-methyl-1H-indol-4-yloxy)acetic acid.]

This example characterized ILY-4001 [2-(3-(2-amino-2-oxoacetyl)-1-(biphenyl-2-ylmethyl)-2-methyl-1H-indol-4-yloxy)acetic acid], alternatively referred to herein as methyl indoxam, with respect to activity, as determined by IC50 assay (Example 1B-1), with respect to cell absorbtion, as determined by in-vitro Caco-2 assay (Example 1B-2) and with respect to bioavailability, as determined using in-vivo mice studies (Example 1B-3).

### Example 1B-1

### IC-50 Study

### ILY-4001 [2-(3-(2-amino-2-oxoacetyl)-1-(biphenyl-2-ylmethyl)-2-methyl-1H-indol-4-yloxy)acetic acid]

This example evaluated the IC50 activity value of ILY-4001 [2-(3-(2-amino-2-oxoacetyl)-1-(biphenyl-2-ylmethyl)-2-methyl-1H-indol-4-yloxy)acetic acid], alternatively referred to herein as methyl indoxam.

A continuous fluorimetric assay for PLA2 activity described in the literature was used to determine IC (Leslie, C C and Gelb, M H (2004) Methods in Molecular Biology “Assaying phospholipase A2 activity”, 284: 229-242, Singer, A G, et al. (2002) Journal of Biological Chemistry “Interfacial kinetic and binding properties of the complete set of human and mouse groups I, II, V, X, and XII secreted phospholipases A2”, 277: 48535-48549, Bezzine, S, et al. (2000) Journal of Biological Chemistry “Exogenously added human group X secreted phospholipase A(2) but not the group IB, IIA, and V enzymes efficiently release arachidonic acid from adherent mammalian cells”, 275: 3179-3191) and references therein.

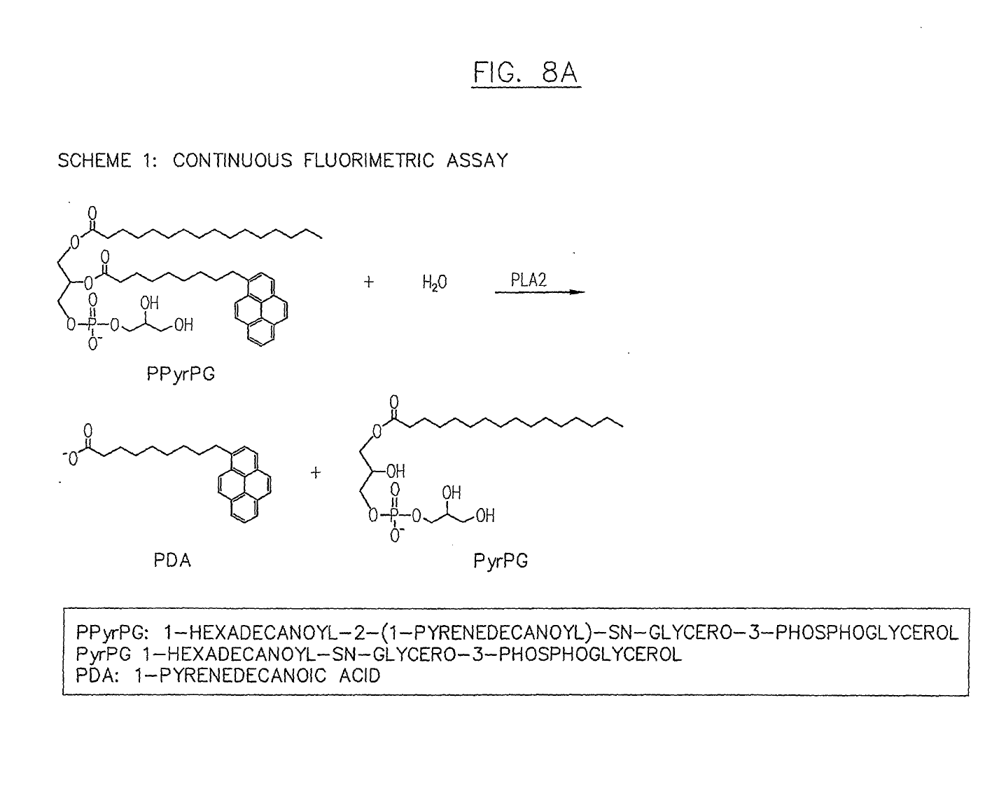

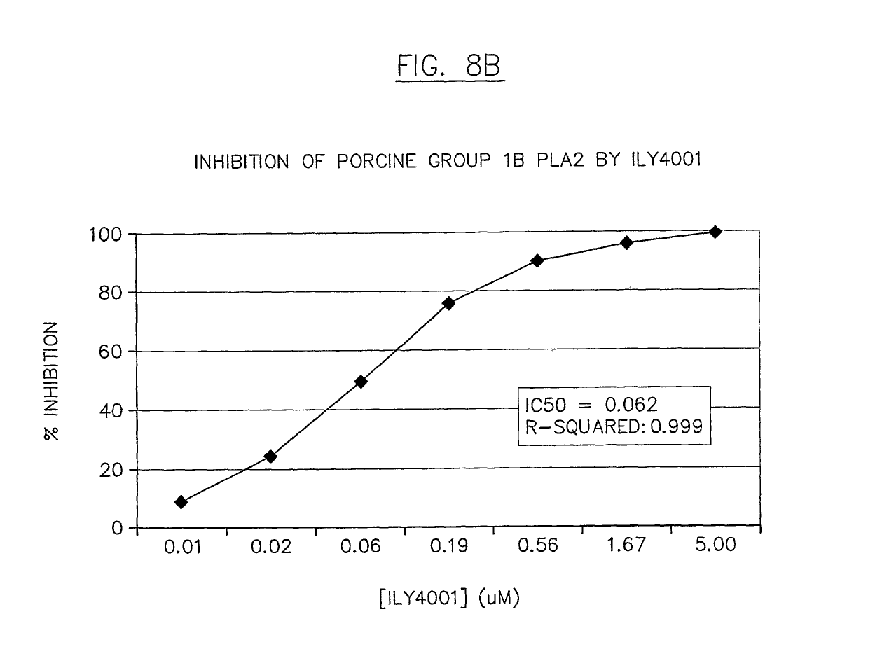

Generally, this assay used a phosphatidylglycerol (or phosphatidylmethanol) substrate with a pyrene fluorophore on the terminal end of the sn-2 fatty acyl chain. Without being bound by theory, close proximity of the pyrenes from neighboring phospholipids in a phospholipid vesicle caused the spectral properties to change relative to that of monomeric pyrene. Bovine serum albumin was present in the aqueous phase and captured the pyrene fatty acid when it is liberated from the glycerol backbone owing to the PLA2-catalyzed reaction. In this assay, however, a potent inhibitor can inhibit the liberation of pyrene fatty acid from the glycerol backbone. Hence, such features allow for a sensitive PLA2 inhibition assay by monitoring the fluorescence of albumin-bound pyrene fatty acid, as represented in Scheme 1 shown in FIG. 8A. The effect of a given inhibitor and inhibitor concentration on any given phospholipase can be determined.

In this example, the following reagents and equipment were obtained from commercial vendors:
1. Porcine PLA2 IB
2. 1-hexadecanoyl-2-(1-pyrenedecanoyl)-sn-glycero-3-phosphoglycerol (PPyrPG)
3. 1-hexadecanoyl-2-(1-pyrenedecanoyl)-sn-glycero-3-phosphomethanol (PPyrPM)
4. Bovine serum albumin (BSA, fatty acid free)
5. 2-Amino-2-(hydroxymethyl)-1,3-propanediol, hydrochloride (Tris-HCl)
6. Calcium chloride
7. Potassium chloride
8. Solvents: DMSO, toluene, isopropanol, ethanol
9. Molecular Devices SPECTRAmax microplate spectrofluorometer
10. Costar 96 well black wall/clear bottom plate

In this example, the following reagents were prepared:
1. PPyrPG (or PPyrPM) stock solution (1 mg/ml) in toluene:isopropanol (1:1)
2. Inhibitor stock solution (10 mM) in DMSO
3. 3% (w/v) bovine serum albumin (BSA)
4. Stock buffer: 50 mM Tris-HCl, pH 8.0, 50 mM KCl and 1 mM CaCl₂

In this example, the procedure was performed as follows:
1. An assay buffer was prepared by adding 3 ml 3% BSA to 47 ml stock buffer.
2. Solution A was prepared by adding serially diluted inhibitors to the assay buffer. Inhibitor were three-fold diluted in a series of 8 from 15 uM.
3. Solution B was prepared by adding PLA2 to the assay buffer. This solution was prepared immediately before use to minimize enzyme activity loss.
4. Solution C was prepared by adding 30 ul PPyrPG stock solution to 90 ul ethanol, and then all 120 ul of PPyrPG solution was transferred drop-wise over approximately 1 min to the continuously stirring 8.82 ml assay buffer to form a final concentration of 4.2 uM PPyrPG vesicle solution.
5. The SPECTRAmax microplate spectrofluorometer was set at 37° C.
6. 100 ul of solution A was added to each inhibition assay well of a costar 96 well black wall/clear bottom plate
7. 100 ul of solution B was added to each inhibition assay well of a costar 96 well black wall/clear bottom plate.
8. 100 ul of solution C was added to each inhibition assay well of a costar 96 well black wall/clear bottom plate.
9. The plate was incubated inside the spectrofluorometer chamber for 3 min.
10. The fluorescence was read using an excitation of 342 nm and an emission of 395 nm.

In this example, the IC50 was calculated using the BioDataFit 1.02 (Four Parameter Model) software package. The equation used to generate the curve fit is:

$${y}_{j} = β + \frac{α - β}{1 + exp ( - κ ( log ( {x}_{j} ) - γ ) )}$$

wherein: α is the value of the upper asymptote; β is the value of the lower asymptote; κ is a scaling factor; γ is a factor that locates the x-ordinate of the point of inflection at

$$exp [ \frac{κ γ - log ( \frac{1 + κ}{κ - 1} )}{κ} ]$$

with constraints α, β, κ, γ>0, β<α, and β<γ<α.

The results, shown in FIG. 8B, indicate that the concentration of ILY4001 resulting in 50% maximal PLA2 activity was calculated to be 0.062 uM.

### Example 1B-2

### Caco-2 Absorbtion Study

### ILY-4001 [2-(3-(2-amino-2-oxoacetyl)-1-(biphenyl-2-ylmethyl)-2-methyl-1H-indol-4-yloxy)acetic acid]

This example evaluated the intestinal absorption of ILY-4001 [2-(3-(2-amino-2-oxoacetyl)-1-(biphenyl-2-ylmethyl)-2-methyl-1H-indol-4-yloxy)acetic acid], alternatively referred to herein as methyl indoxam using in-vitro assays with Caco-2 cells.

Briefly, the human colon adenocarcinoma cell line, Caco-2, was used to model intestinal drug absorption. It has been shown that the apparent permeability values measured in Caco-2 monolayers in the range of 1×10⁻⁷ cm/sec or less typically correlate with relatively poor human absorption. (Artursson, P., K. Palm, et al. (2001). “Caco-2 monolayers in experimental and theoretical predictions of drug transport.” Adv Drug Deliv Rev 46(1-3): 27-43.).

In order to determine the compound permeability, Caco-2 cells (ATCC) were seeded into 24-well transwells (Costar) at a density of 6×10⁴ cells/cm². Monolayers were grown and differentiated in MEM (Mediatech) supplemented with 20% FBS, 100 U/ml penicillin, and 100 ug/ml streptomycin at 37° C., 95% humidity, 95% air, and 5% CO₂. The culture medium was refreshed every 48 hours. After 21 days, the cells were washed in transport buffer made up of HBSS with HEPES and the monolayer integrity was evaluated by measuring the trans-epithelial electrical resistance (TEER) of each well. Wells with TEER values of 350 ohm-cm² or better were assayed.

ILY-4001 and Propranolol (a transcellular transport control) were diluted to 50 ug/ml in transport buffer and added to the apical wells separately. 150 ul samples were collected for LC/MS analysis from the basolateral well at 15 min, 30 min, 45 min, 1 hr, 3 hr, and 6 hr time points; replacing the volume with pre-warmed transport buffer after each sampling. The apparent permeabilities in cm/s were calculated based on the equation:
Pₐₚₚ=(dQ/dt)×(1/C₀)×(1/A)
Where dQ/dt is the permeability rate corrected for the sampling volumes over time, C₀ is the initial concentration, and A is the surface area of the monolayer (0.32 cm²). At the end of the experiment, TEER measurements were retaken and wells with readings below 350 ohm-cm² indicated diminished monolayer integrity such that the data from these wells were not valid for analysis. Finally, wells were washed with transport buffer and 100 uM of Lucifer Yellow was added to the apical wells. 15 min, 30 min, and 45 min time points were sampled and analyzed by LC/MS to determine paracellular transport.

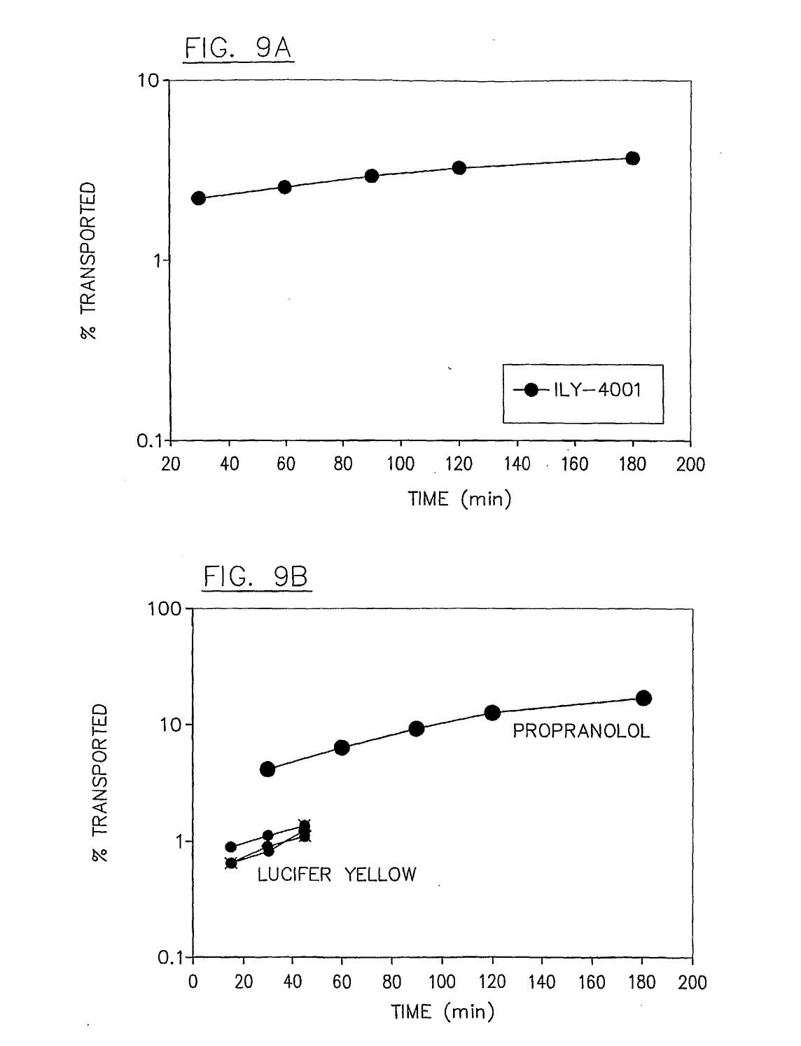

Results from the Caco-2 permeability study for ILY-4001 are shown in FIG. 9A, in which the apparent permeability (cm/s) for ILY-4001 was determined to be around 1.66×10⁻⁷. The results for Lucifer Yellow and Propranolol permeability as paracellular and transcellular transport controls were also determined, and are shown in FIG. 9B, with determined apparent permeability (cm/s) of around 1.32×10⁻⁵ for

Propranolol and around 2.82×10⁻⁷+/−0.37×10⁻⁷ for Lucifer Yellow.

### Example 1B-3

### Pharmokinetic Study

### ILY-4001 [2-(3-(2-amino-2-oxoacetyl)-1-(biphenyl-2-ylmethyl)-2-methyl-1H-indol-4-yloxy)acetic acid]

This example evaluated the bioavailability of ILY-4001 [2-(3-(2-amino-2-oxoacetyl)-1-(biphenyl-2-ylmethyl)-2-methyl-1H-indol-4-yloxy)acetic acid], alternatively referred to herein as methyl indoxam. Specifically, a pharmokinetic study was conducted to determine the fraction of unchanged ILY-4001 in systemic circulation following administration.

Bioavailability was calculated as a ratio of AUC-oral/AUC-intravenous (IV). To determine this ratio, a first set of subject animals were given a measured intravenous (IV) dose of ILY-4001, followed by a determination of ILY-4001 levels in the blood at various time points after administration (e.g., 5 minutes through 24 hours). Another second set of animals was similarly dosed using oral administration, with blood levels of ILY-4001 determined at various time points after administration (e.g., 30 minutes through 24 hours). The level of ILY-4001 in systemic circulation were determined by generally accepted methods (for example as described in Evans, G., A Handbook of Bioanalysis and Drug Metabolism. Boca Raton, CRC Press (2004)). Specifically, liquid scintillation/mass spectrometry/mass spectrometry (LC/MS/MS) analytical methods were used to quantitate plasma concentrations of ILY-4001 after oral and intravenous administration. Pharmacokinetic parameters that were measured include Cₘₐₓ, AUC, tₘₐₓ, t_{1/2}, and F (bioavailability).

In this procedure, ILY-4001 was dosed at 3 mg/kg IV and 30 mg/kg oral. The results of this study, summarized in Table 4, showed a measured bioavailability of 28% of the original oral dose. This indicated about a 72% level of non-absorption of ILY-4001 from the GI tract into systemic circulation.

TABLE 4
Results of Pharmokinetic Study for ILY-4001

| | IV | ORAL |
|---|---|---|
| t½ (h) | 1.03 | 1.25 |
| Cmax (ng/mL) | 3168 | 2287 |
| Tmax (h) | 0.083 | 1 |
| AUC 0-24) (h * ng/mL) | 2793 | 5947 |
| AUC(0-inf) (h * ng/mL) | 2757 | 5726 |
| % F | | 28.0 |

### Example 1C

### Charge Modification of ILY-4001 to Improve Lumen-Localization

### Synthesis of 3-(3-aminooxalyl-1-biphenyl-2-yl methyl-4-carboxymethoxy-2-methyl-1H-indol-5-yl)-propionic acid

This example describes an approach for charge modification of ILY-4001 [2-(3-(2-amino-2-oxoacetyl)-1-(biphenyl-2-ylmethyl)-2-methyl-1H-indol-4-yloxy)acetic acid], alternatively referred to herein as methyl indoxam, to improve lumen-localization thereof. Specifically, ILY-4001 can be modified at certain substituent groups, including for example to change the ionic charge, and to impart improved lumen-localization. In this example, a scheme is presented by which ILY-4001 can be modified to add a propanoic acid moiety at position 5 (as shown in FIG. 5) to form 3-(3-aminooxalyl-1-biphenyl-2-ylmethyl-4-carboxymethoxy-2-methyl-1H-indol-5-yl)-propionic acid.

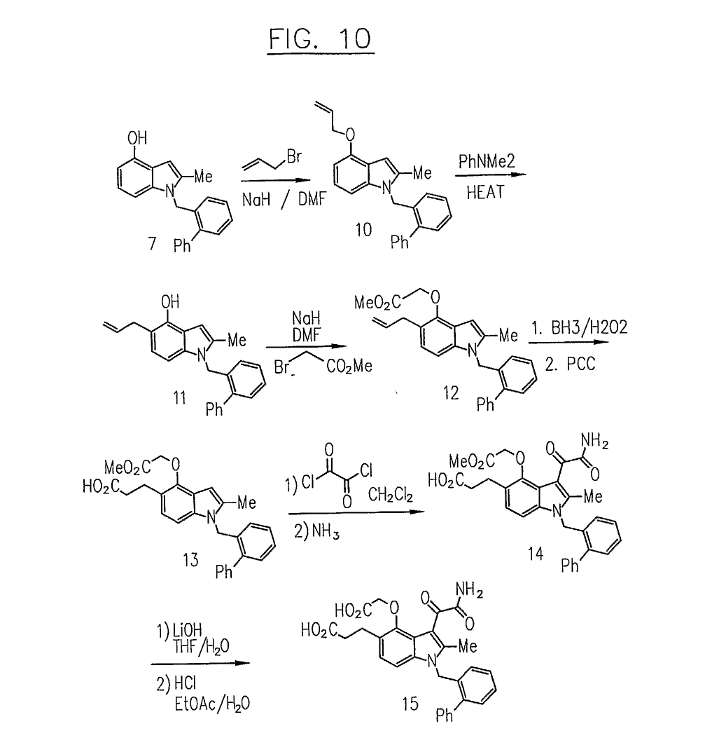

Reference is made to FIG. 10, which outlines the overall synthesis scheme to prepare 3-(3-aminooxalyl-1-biphenyl-2-ylmethyl-4-carboxymethoxy-2-methyl-1H-indol-5-yl)-propionic acid. The numbers under each compound shown in FIG. 10 correspond to the numbers in parenthesis associated with the chemical name for each compound in the following experimental description. The starting compound as shown in FIG. 10 (indicated with parenthetical (7)) can be prepared as shown in FIG. 7 and described in connection with Example 1A.

A solution of 1.0 g (4 mmol) of 7 in 10 mL of THF and 75 mL of DMF is stirred with 200 mg of NaH (60% in mineral oil; 5 mmol) for 10 min, and then with 0.4 mL (4.6 mmol) of allyl bromide for 2 h. The solution is diluted with water and extracted with EtOAc. The organic phase is washed with brine, dried over Na₂SO₄, evaporated at reduced pressure, and purified by column chromatography to obtain compound 10. This material is heated at reflux in 20 mL of N,N-dimethylaniline for 19 h, cooled, diluted with EtOAc, washed with 1 N HCl, H₂O, and brine, dried (Na₂SO₄), concentrated, and purified by column chromatography to obtain compound 11. This material (3.4 mmol) is dissolved in 60 mL of DMF and 10 mL of THF, 150 mg of NaH (60% in mineral oil; 3.7 mmol) is added, the mixture is stirred for 15 min, 0.4 mL (3.6 mmol) of ethyl bromoacetate is added, and stirring is continued for an additional 2.5 h. The solution is diluted with water and extracted with EtOAc. The organic phase is washed with brine, dried (Na₂SO₄), evaporated at reduced pressure, and purified by column chromatography to obtain compound 12. To a solution of 12 (0.022 mmol) in anhyd THF (1 mL) at r.t. is added BH3.THF (0.44 mL) complex (2.0 equiv, 1 M solution in THF, 0.044 mmol). The reaction mixture is stirred for 2 h at r.t., and is quenched carefully with drop wise addition of excess of 30% aq hydrogen peroxide and 15% aq NaOH. The mixture is then stirred vigorously for 30 min at r.t. The resultant mixture is was extracted, evaporated, and purified by column chromatography. The obtained alcohol in THF is added dropwisely to PCC solution and stirred for 3 hours. The reaction mixture is then purified to obtain compound 13. To a stirred solution of oxalyl chloride (1.2 mmol) in anhydrous CH₂Cl₂ (4 mL), a solution of compound 13 in CH₂Cl₂ (4 mL) is added drop wise and the reaction mixture is stirred for 80 min at RT. After cooling the reaction mixture to −10° C., a saturated solution of NH₃ in CH₂Cl₂ (10 mL) is added drop wise and then the reaction mixture is saturated with NH₃ (gas) at ca. 0° C. The reaction mixture is allowed to warm up to RT and is concentrated under reduced pressure to dryness and purified by column chromatography to obtain compound 14. To a stirred solution of compound 14 (1 mmol) in a mixture of THF (5 mL) and water (1 mL), a solution of lithium hydroxide monohydrate (2 mmol) in water (2 mL) is added portion wise and the reaction mixture is stirred for 2 h at RT. After addition of water (7 mL), the reaction mixture is concentrated under reduced pressure to a volume of ca. 100 mL. Then, to the residual yellow slurry, 2N HCl (2 mL) and EtOAc (20 mL) is added, the resultant mixture is stirred for 24 h at RT, and followed by column chromatography to obtain compound 15.

### Example 1D

### Synthesis of Polymer-Linked ILY-4001 to Improve Lumen-Localization

### Synthesis of random copolymer of [3-Aminooxalyl-2-methyl-1-(2′-vinyl-biphenyl-2-ylmethyl)-1H-indol-4-yloxy]-acetic acid, styrene, and styrene sulfonic acid sodium salt

This example describes approaches for synthesizing a phospholipase inhibitor comprising an oligomer or polymer moiety covalently linked to ILY-4001 [2-(3-(2-amino-2-oxoacetyl)-1-(biphenyl-2-ylmethyl)-2-methyl-1H-indol-4-yloxy)acetic acid], alternatively referred to herein as methyl indoxam, to improve lumen-localization thereof. Specifically, ILY-4001 was polymer linked to impart improved lumen-localization. In this example, a scheme is presented by which ILY-4001 can be linked to a random co-polymer to form to form a random copolymer of [3-Aminooxalyl-2-methyl-1-(2′-vinyl-biphenyl-2-ylmethyl)-1H-indol-4-yloxy]-acetic acid, styrene, and styrene sulfonic acid sodium salt.

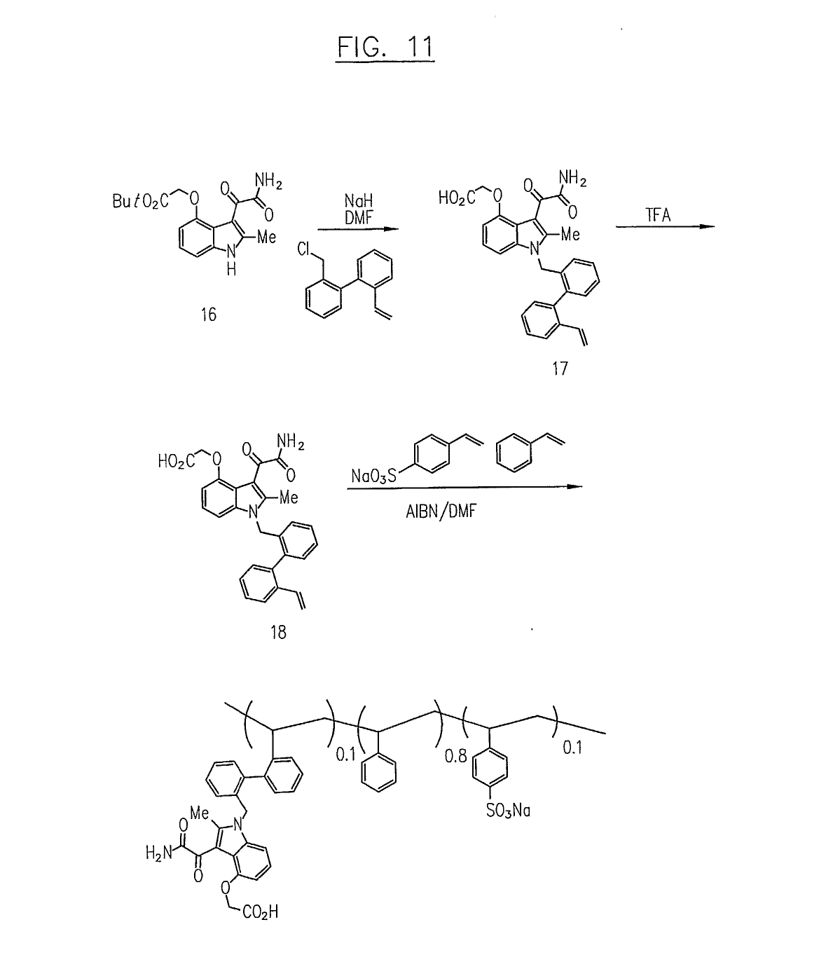

Referring to FIG. 11, the overall synthesis scheme for is outlined for polymer-linked ILY-4001. The numbers under each compound shown in FIG. 11 correspond to the numbers in parenthesis associated with the chemical name for each compound in the following experimental description. The starting compound as shown in FIG. 11 (indicated with parenthetical (16)) can be obtained from literature.

Compound 16 obtained from literature procedure (Bioorg. Med. Chem., 2004, 12, 1737-1749.) (0.10 mol) in anhydrous DMF (100 mL) is added drop wise to a stirred cooled (ca. 15° C.) suspension of sodium hydride (0.15 mol, 6.0 g, 60% in mineral oil, washed with 100 mL of hexanes before the reaction) in DMF (50 mL) and the reaction mixture is stirred for 0.5 h at RT. After cooling the reaction mixture to ca. 5° C., 2-(2-vinyl phenyl)benzyl chloride (0.101 mol) is added drop wise and the reaction mixture is stirred for 18 h at RT. The reaction is quenched with water (10 mL) and EtOAc (500 mL) is added. The resulted mixture is washed with water, brine, and dried over MgSO₄. After filtration and removal of the solvent from the filtrate under reduced pressure, the residue is purified by dry chromatography to afford product 17. To the solution of (1 mmol) of 17 in 15 mL of CH₂Cl₂ is added 2 mL of trifluoroacetic acid. This mixture is stirred for 1.5 hour, the solvent is evaporated at reduced pressure, and the residue is diluted with EtOAc and water. The organic phase is washed with brine, dried over MgSO₄, evaporated at reduced pressure, and purified by column chromatography to obtain compound 18. A mixture of 18, styrene sulfonic acid sodium salt, and styrene in mole ratio of 1:1:8 (in total one mmol) is dissolved in 2 mL of a mixed solvent (water/DMF=2/8 v/v). To the mixture AIBN (2,2′-azobisisobutyronitrile, 0.01 mmol) is added. The resulted solution is heated to 75° C. for 16 hours. After the reaction is cooled to rt, it is precipitated into iso-propyl alcohol twice, and dried under reduced pressure to obtain the co-polymer.

### Example 2

### Linking to Inhibitor Moieties

### Synthesis of [3-Aminiooxalyl-2-methyl-1-(4-vinyl-benzyl)-1H-indol-4-yloxy]-acetic acid (21)

### Synthesis of (1-Acryloyl-3-aminooxalyl-2-methyl-1H-indol-4-yloxy)-acetic acid (23)

### Synthesis of (3-Aminooxalyl-2-methyl-1-[2-(pyrazole-1-carbothioylsulfanyl)propionyl]-1H-indol-4-yloxy-3-acetic acid (26)

This example describes approaches for covalently linking a phospholipase inhibiting moiety to linking moieties.

ILY-4001 [2-(3-(2-amino-2-oxoacetyl)-1-(biphenyl-2-ylmethyl)-2-methyl-1H-indol-4-yloxy)acetic acid], alternatively referred to herein as methyl indoxam, can be linked to various linking moieties (as a first step in a process to form compounds having improved lumen-localization thereof). In this example, a scheme is presented by which ILY-4001 can be provided with linking groups to form [3-Aminooxalyl-2-methyl-1-(4-vinyl-benzyl)-1H-indol-4-yloxy]-acetic acid (21); Synthesis of (1-Acryloyl-3-aminooxalyl-2-methyl-1H-indol-4-yloxy)-acetic acid (23); Synthesis of {3-Aminooxalyl-2-methyl-1-[2-(pyrazole-1-carbothioylsulfanyl)propionyl]-1H-indol-4-yloxy}-acetic acid (26).

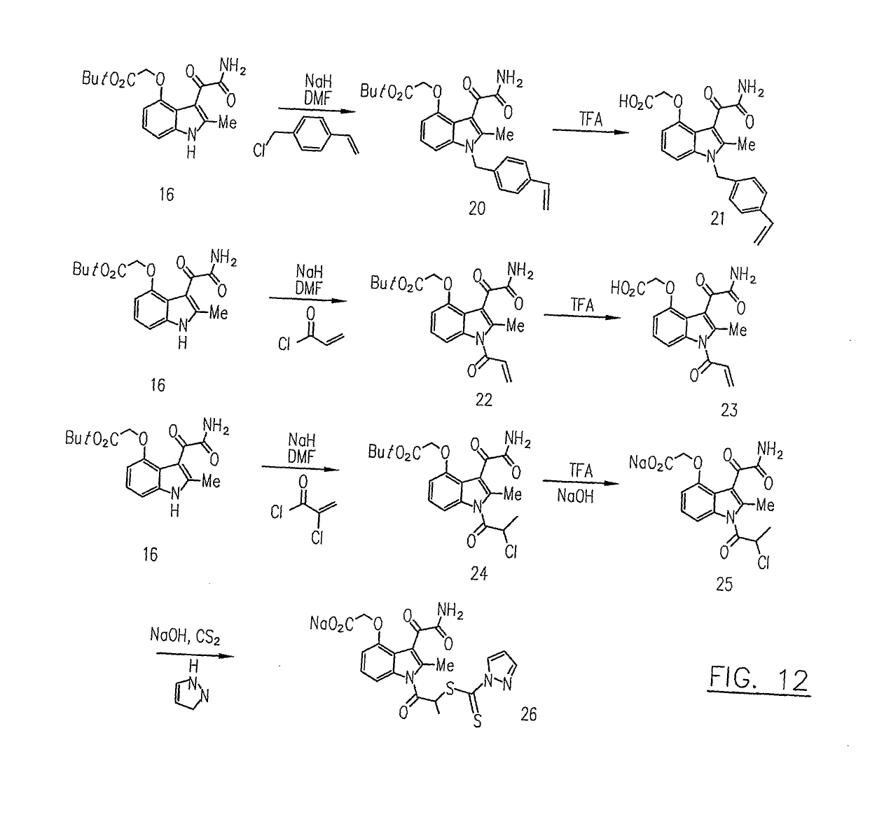

Referring to FIG. 12, the overall synthesis scheme for is outlined for preparing ILY-4001 with various linking groups. The numbers under each compound shown in FIG. 12 correspond to the numbers in parenthesis associated with the chemical name for each compound in the following experimental description. The starting compound as shown in FIG. 12 (indicated with parenthetical (16)) can be obtained from literature.

Compound 16 (0.10 mol) in anhydrous DMF (100 mL) is added drop wise to a stirred cooled (ca. 15° C.) suspension of sodium hydride (0.15 mol, 6.0 g, 60% in mineral oil, washed with 100 mL of hexanes before the reaction) in DMF (50 mL) and the reaction mixture is stirred for 0.5 h at RT. After cooling the reaction mixture to ca. 5° C., 4-vinyl benzyl chloride (0.101 mol) is added drop wise and the reaction mixture is stirred for 18 h at RT. The reaction is quenched with water (10 mL) and EtOAc (500 mL) is added. The resulted mixture is washed with water, brine, and dried over MgSO₄. After filtration and removal of the solvent from the filtrate under reduced pressure, the residue is purified by dry chromatography to afford product 20. To the solution of (1 mmol) of 20 in 15 mL of CH₂Cl₂ is added 2 mL of trifluoroacetic acid. This mixture is stirred for 1.5 hour, the solvent is evaporated at reduced pressure, and the residue is diluted with EtOAc and water. The organic phase is washed with brine, dried over MgSO₄, evaporated at reduced pressure, and purified by column chromatography to obtain compound 21.

A similar procedure is used to prepare compound 23.

A 100 mL round-bottomed flask equipped with a magnetic stirring bar and a PE stopper is charged with pyrazole (3 mmol), sodium hydroxide (0.12 g) and DMSO (5 mL) at ambient temperature (25° C.). Carbon disulfide (0.180 mL) is added to the flask dropwise. The mixture is further stirred for one hour. Compound 25 in DMSO obtained from the similar preceding procedure after treated with NaOH solution is then added to the reaction mixture slowly. The reaction is stirred for 2 hours. The solution is poured into 100 mL water is extracted with ethyl acetate. The organic layer is further washed with water (2×100 mL) and dried over MgSO₄. The solvent is removed under reduced pressure and the product is further purified by flash column chromatography.

### Example 3

### Synthesis of Polymer-Linked Inhibitors

This example describes approaches for preparing polymer-linked inhibitors comprising an oligomer or polymer moiety covalently linked to an inhibiting moiety, where the polymer moiety is a soluble random co-polymer (Example 3A), or an insoluble cross-linked random copolymer (Example 3B).

### Example 3A

### Synthesis of Polymer-Linked Inhibitors with Soluble Random Copolymer

### Synthesis of copolymer of (-Acryloyl-3-aminooxalyl-2-methyl-1H-indol-4-yloxy)-acetic acid (23) and dimethyl acrylamide

In this example, approaches are outlined for synthesizing a phospholipase inhibitor comprising an oligomer or polymer moiety covalently linked to an inhibiting moiety, where the polymer moiety is a soluble random co-polymer. Specifically, a scheme is provided for synthesizing a copolymer of (1-Acryloyl-3-aminooxalyl-2-methyl-1H-indol-4-yloxy)-acetic acid (23) and dimethyl acrylamide.

A starting compound for this example can be from compound 23 having a linking group prepared as described in connection with Example 2. The polymer formed can be represented by the schematic chemical formula: 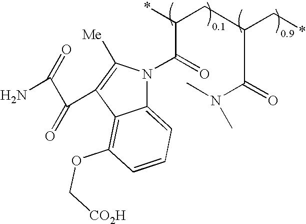
Briefly, a mixture of 23 and dimethyl acrylamide in mole ratio of 1:9 (in total one mmol) is dissolved in 2 mL of isopropanol. To the mixture AIBN (2,2′-azobisisobutyronitrile 0.01 mmol) is added. The resulted solution is heated to 75° C. for 8 hours. After the reaction is cooled to rt, it is diluted with 100 mL of water and dialyzed against water for 48 hours. The solution then is freeze-dried to obtain the co-polymer.

### Example 3B

### Synthesis of Polymer-Linked Inhibitors with Insoluble (Cross-Linked) Random Copolymer

### Synthesis of random copolymer of [3-Aminooxalyl-2-methyl-1-(4-vinyl-benzyl)-1H-indol-4-yloxy]-acetic acid (21), styrene, and styrene sulfonic acid sodium salt, crosslinked with divinyl benzene

This example describes approaches for synthesizing a phospholipase inhibitor comprising an oligomer or polymer moiety covalently linked to an inhibiting moiety, where the polymer moiety is an insoluble, cross-linked random co-polymer. Specifically, a scheme is provided for synthesizing a copolymer of [3-Aminooxalyl-2-methyl-1-(4-vinyl-benzyl)-1H-indol-4-yloxy]-acetic acid (21), styrene, and styrene sulfonic acid sodium salt, crosslinked with divinyl benzene.

A starting compound for this example can be from compound 21 having a linking group prepared as described in connection with Example 2. The polymer formed can be represented by the schematic chemical formula:

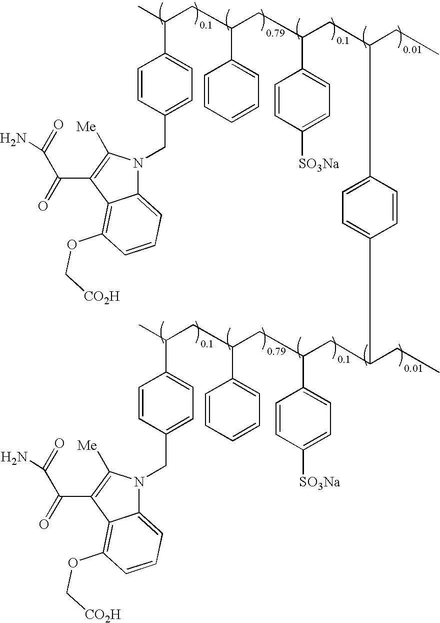

A mixture of 21, styrene sulfonic acid sodium salt, styrene, divinyl benzene in mole ratio of 1:1:7.9:0.1 (in total 10 mmol) is dissolved in 20 mL of a mixed solvent (water/DMF=2/8 v/v). To the mixture AIBN (2,2′-azobisisobutyronitrile 0.1 mmol) is added. The resulted solution is heated to 75° C. for 24 hours. After the reaction is cooled to rt, the resulted crosslinked solid material is mechanically milled into find gel, washed with excess amount of water, dried under reduced pressure to obtain the co-polymer.

### Example 4

### Synthesis of Polymer-Linked Inhibitors by Polymer-Particle Modification

### Synthesis of (3-Aminooxalyl-1-dodecyl-2-methyl-1H-indol-4-yloxy)-acetic acid modified Cavilink™ bead

This example describes approaches for synthesizing a phospholipase inhibitor comprising an oligomer or polymer moiety covalently linked to an inhibiting moiety, where the polymer moiety is an insoluble particle, and the inhibiting moiety is linked to the particle. Specifically, a scheme is provided for synthesis of (3-Aminooxalyl-1-dodecyl-2-methyl-1H-indol-4-yloxy)-acetic acid modified Cavilink™ bead.

The polymer formed can be represented by the schematic representation: 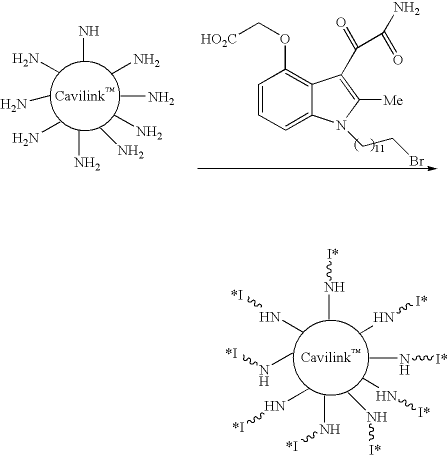
Commercial available polystyrene Cavilink™ Bead (1 g) is suspended in ethanol at rt. To the solution, the inhibitor compound (100 mg) (shown above the arrow as a reactant; represented as “I” in the product compound) is added and stirred for 24 hours. The bead is filtered and washed with excess of ethanol until no detection of inhibitor by UV. The bead then is dried under reduced pressure.

### Example 5

### Synthesis of Polymer-Linked Inhibitors with Graft Copolymers

### Synthesis of star copolymer of (1-Acryloyl-3-aminooxalyl-2-methyl-1H-indol-4-yloxy)-acetic acid, n-butyl acrylate, dimethyl acrylamide, and N-(2-Acryloylamino-ethyl)-acrylamide

This example describes approaches for synthesizing a phospholipase inhibitor comprising an oligomer or polymer moiety covalently linked to an inhibiting moiety, where the polymer moiety is linked using graft copolymers. In particular, a scheme is provided for synthesis of a star copolymer of (1-Acryloyl-3-aminooxalyl-2-methyl-1H-indol-4-yloxy)-acetic acid, n-butyl acrylate, dimethyl acrylamide, and N-(2-Acryloylamino-ethyl)-acrylamide.

The synthesis scheme and the polymer formed thereby can be represented by the schematic representation: 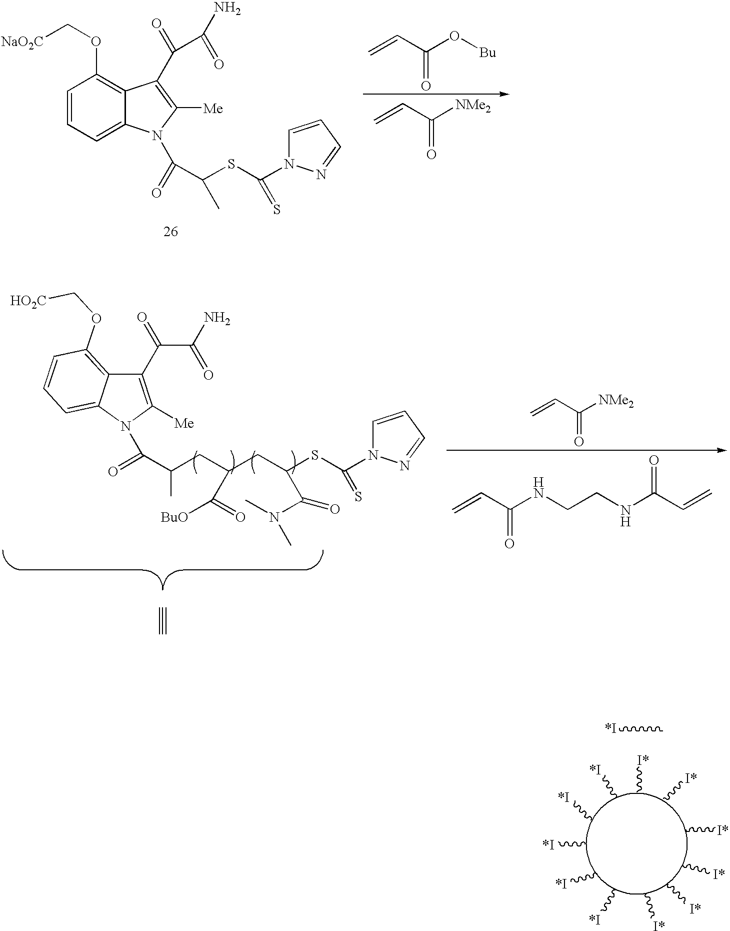
A mixture of 26, dimethyl acrylamide, and n-butyl acrylate in a mole ratio of 0.04:0.48:0.48 (in total 10 mmol) is dissolved in 20 mL of DMF. To the mixture AIBN (2,2′-azobisisobutyronitrile, 10 mmol % to compound 26) is added and is heated to 75° C. for 8 hours. To the resulted yellow solution 1 mmol of dimethyl acrylamide and ethylene bis-diacrylamide (1:1) is added and stirred for an additional 8 hours. After the reaction is cooled to rt, the reaction mixture is precipitated twice, dried under reduced pressure to obtain the co-polymer.

### Example 6A

### Synthesis of Tailored-Polymer-Singlet

### Synthesis of poly-n-butyl acrylate tailored (1-Acryloyl-3-aminooxalyl-2-methyl-1H-indol-4-yloxy)-acetic acid

This example describes approaches for synthesizing a phospholipase inhibitor comprising an oligomer or polymer moiety covalently linked to a single inhibiting moiety to form a phospholipase inhibitor “singlet”. Specifically, a scheme is provided for synthesis of poly-n-butyl acrylate tailored (1-Acryloyl-3-aminooxalyl-2-methyl-1H-indol-4-yloxy)-acetic acid.

The synthesis scheme and the polymer formed thereby can be represented by the schematic representation: 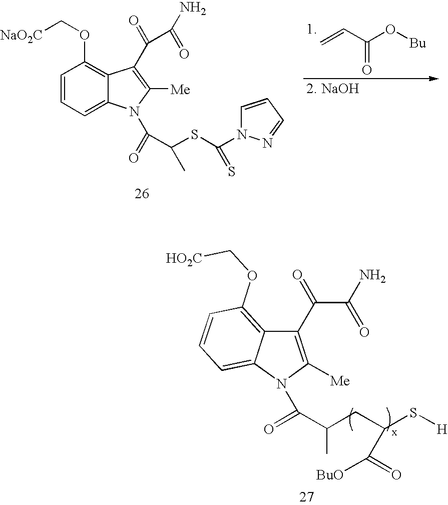
A mixture of 26 and n-butyl acrylate in a mole ratio of 0.04:0.96 (in total 10 mmol) is dissolved in 20 mL of DMF. To the mixture AIBN (2,2′-azobisisobutyronitrile, 10 mmol % to compound 26) is added and is heated to 75 for 16 hours. After the reaction is cooled to 45° C., to the resulted yellow solution 2 mL of 10% NaOH solution is added and stirred for an additional 8 hours. After the reaction is cooled to rt, the reaction mixture is precipitated twice, dried under reduced pressure to obtain the co-polymer.

### Example 6B

### Synthesis of Tailored-Polymer-Dimers

This example describes various approaches for synthesizing a phospholipase inhibitor comprising an oligomer or polymer moiety covalently linked to two inhibiting moieties to form a phospholipase inhibitor “dimer”. Specifically, in a first approach, a scheme for the synthesis of disulfide dimer of poly-n-butyl acrylate tailored (1-Acryloyl-3-aminooxalyl-2-methyl-1H-indol-4-yloxy)-acetic acid is disclosed (Example 6B-1). In a second approach, a scheme for the synthesis of (3-Aminooxalyl-1-{12-[12-(3-aminooxalyl-4-carboxymethoxy-2-methyl-indol-1-yl)-dodecyldisulfanyl]-dodecyl}-2-methyl-1H-indol-4-yloxy)-acetic acid (31).

### Example 6B-1

### Synthesis of Tailored-Polymer-Dimer

### Synthesis of disulfide dimer of poly-n-butyl acrylate tailored (1-Acryloyl-3-aminooxalyl-2-methyl-1H-indol-4-yloxy)-acetic acid

The synthesis scheme and the polymer formed thereby can be represented by the schematic representation: 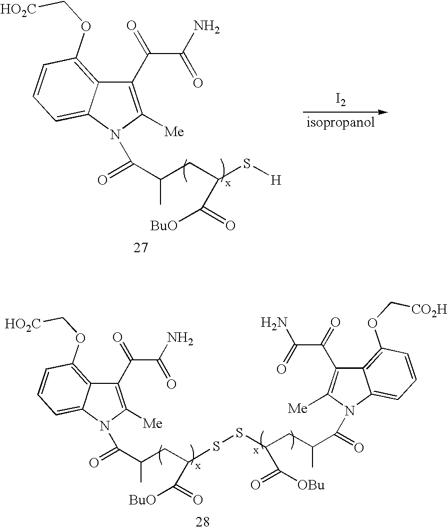
To a solution of 27 (1 mmol) in isopropanol (10 mL) is added iodine (127 mg, 0.5 mmol). After 2 hours, the reaction mixture is concentrated and redissolved in EtOAc (25 mL). the solution is washed with Na₂S₂O₄ (2×10 mL) and brine (10 mL), dried over sodium sulfate, filtered, and concentrated in vacuo. The product was purified by precipitation to provide disulfide 28

### Example 6B-1

### Synthesis of Tailored-Polymer-Dimer

### Synthesis of (3-Aminooxalyl-1-{12-[12-(3-aminooxalyl-4-carboxymethoxy-2-methyl-indol-1-yl)-dodecyldisulfanyl]-dodecyl}-2-methyl-1H-indol-4-yloxy)-acetic acid (31)

The synthesis scheme and the polymer formed thereby can be represented by the schematic representation:

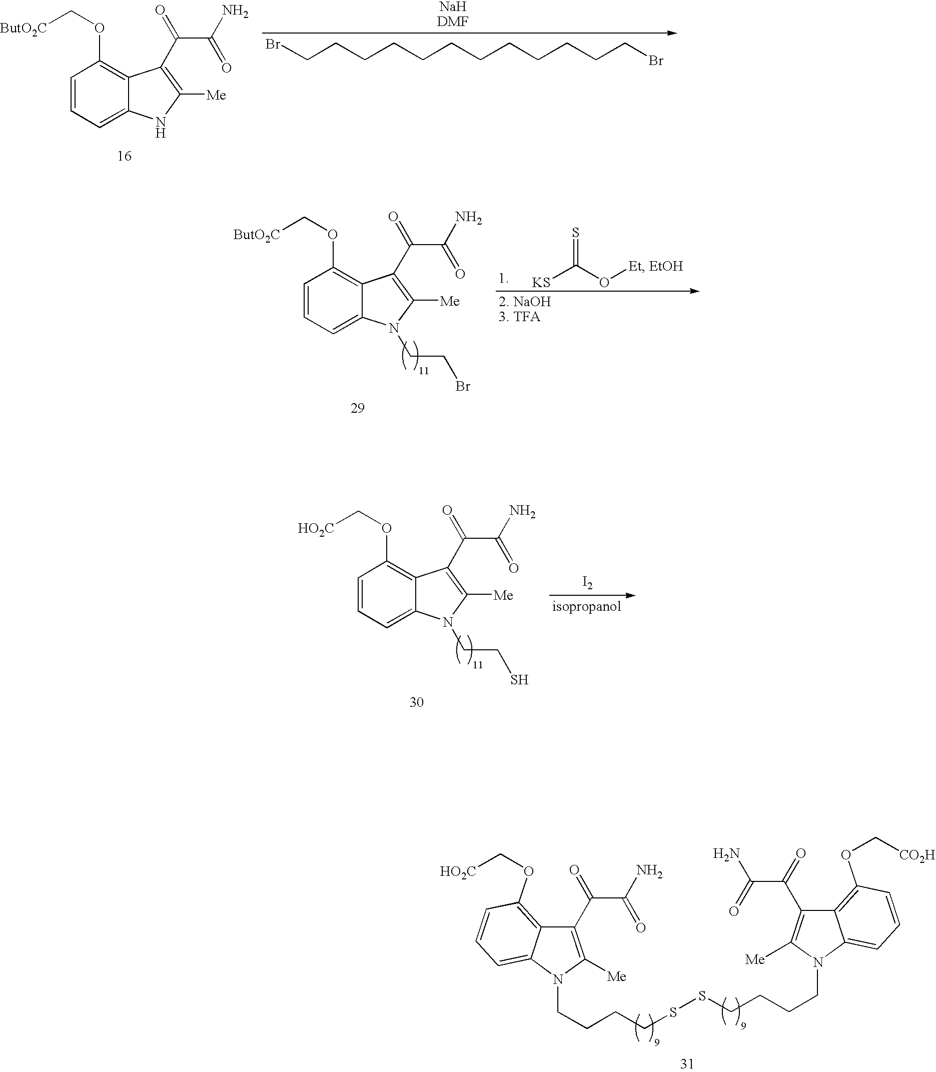

Compound 16 (10 mol) in anhydrous DMF (100 mL) is added drop wise to a stirred cooled (ca. 15° C.) suspension of sodium hydride (0.015 mol, 600 mg, 60% in mineral oil, washed with 10 mL of hexanes before the reaction) in DMF (50 mL) and the reaction mixture is stirred for 0.5 h at RT. After cooling the reaction mixture to ca. 5° C., 1,12-dibromododecane (10.1 mmol) is added at once and the reaction mixture is stirred for 18 h at RT. The reaction is quenched with water (10 mL) and EtOAc (500 mL) is added. The resulted mixture is washed with water, brine, and dried over MgSO₄. After filtration and removal of the solvent from the filtrate under reduced pressure, the residue is purified by dry chromatography to afford product 29. To the solution of (1 mmol) of 29 in 30 mL of EtOH is added 1.1 mmol of dithiocarbonic acid ethyl ester potassium salt. This mixture is stirred for 12 hour and then the reaction is heated to 45° C. To the resulted yellow solution 2 mL of 10% NaOH solution is added and stirred for an additional 8 hours. After the reaction is cooled to rt, solvent is removed and extracted with EtOAc. The resulted mixture is washed with water, brine, and dried over MgSO₄ to obtain a crude product. To the solution of (1 mmol) of the crude product in 15 mL of CH₂Cl₂ is added 2 mL of trifluoroacetic acid. This mixture is stirred for 1.5 hour, the solvent is evaporated at reduced pressure, and the residue is diluted with EtOAc and water. The organic phase is washed with brine, dried over MgSO₄, evaporated at reduced pressure, and purified by column chromatography to obtain compound 30. To a solution of 30 (1 mmol) in isopropanol (10 mL) is added iodine (127 mg, 0.5 mmol). After 2 hours, the reaction mixture is concentrated and redissolved in EtOAc (25 mL). the solution is washed with Na₂S₂O₄ (2×10 mL) and brine (10 mL), dried over sodium sulfate, filtered, and concentrated in vacuo. The product was purified by column chromatography to provide disulfide 31.

### Example 7

### Reduction in Insulin Resistance in a Mouse Model

A phospholipase inhibitor, for example a composition comprising a phospholipase inhibiting moiety disclosed herein, can be used in a mouse model to demonstrate, for example, suppression of diet-induced insulin resistance, relating to, for example, diet-induced onset of diabetes. The phospholipase inhibitor can be administered to subject animals either as a chow supplement and/or by oral gavage BID in a certain dosage (e.g., less than about 1 ml/kg body weight, or about 25 to about 50 μl/dose). A typical vehicle for inhibitor suspension comprises about 0.9% carboxymethylcellulose, about 9% PEG-400, and about 0.05% Tween 80, with an inhibitor concentration of about 5 to about 13 mg/ml. This suspension can be added as a supplement to daily chow, e.g., less than about 0.015% of the diet by weight, and/or administered by oral gavage BID, e.g., with a daily dose of about 10 mg/kg to about 90 mg/kg body weight.

The mouse chow used may have a composition representative of a Western (high fat and/or high cholesterol) diet. For example, the chow may contain about 21% milk fat and about 0.15% cholesterol by weight in a diet where 42% of total calories are derived from fat. See, e.g., Harlan Teklad, diet TD88137. When the inhibitor is mixed with the chow, the vehicle, either with or without the inhibitor, can be mixed with the chow and fed to the mice every day for the duration of the study.

The duration of the study is typically about 6 to about 8 weeks, with the subject animals being dosed every day during this period. Typical dosing groups, containing about 6 to about 8 animals per group, can be composed of an untreated control group, a vehicle control group, and dose-treated groups ranging from about 10 mg/kg body weight to about 90 mg/kg body weight.

At the end of the about 6 to about 8 week study period, an oral glucose tolerance test and/or an insulin sensitivity test can be conducted as follows:

Oral glucose tolerance test—after an overnight fast, mice from each dosing group can be fed a glucose bolus (e.g., by stomach gavage using about 2 g/kg body weight) in about 50 μl of saline. Blood samples can be obtained from the tail vein before, and about 15, about 30, about 60, and about 120 minutes after glucose administration; blood glucose levels at the various time points can then be determined.

Insulin sensitivity test—after about a 6 hour morning fast, mice in each of the dosing groups can be administered bovine insulin (e.g., about 1 U/kg body weight, using, e.g., intraperitoneal administration. Blood samples can be obtained from the tail vein before, and about 15, about 30, about 60, and about 120 minutes after insulin administration; plasma insulin levels at the various time points can then be determined, e.g., by radioimmunoassay.

The effect of the non-absorbed phospholipase inhibitor, e.g., a phospholipase A2 inhibitor, is a decrease in insulin resistance, i.e., better tolerance to glucose challenge by, for example, increasing the efficiency of glucose metabolism in cells, and in the animals of the dose-treated groups fed a Western (high fat/high cholesterol) diet relative to the animals of the control groups. Effective dosages can also be determined.

### Example 8

### Reduction in Fat Absorption in a Mouse Model

A phospholipase inhibitor, for example a composition comprising a phospholipase inhibiting moiety disclosed herein, can be used in a mouse model to demonstrate, for example, reduced lipid absorption in subjects on a Western diet. The phospholipase inhibitor can be administered to subject animals either as a chow supplement and/or by oral gavage BID in a certain dosage (e.g., less than about 1 ml/kg body weight, or about 25 to about 50 μl/dose). A typical vehicle for inhibitor suspension comprises about 0.9% carboxymethylcellulose, about 9% PEG-400, and about 0.05% Tween 80, with an inhibitor concentration of about 5 to about 13 mg/ml. This suspension can be added as a supplement to daily chow, e.g., less than about 0.015% of the diet by weight, and/or administered by oral gavage BID, e.g., with a daily dose of about 10 mg/kg to 90 mg/kg body weight.

The mouse chow used may have a composition representative of a Western-type (high fat and/or high cholesterol) diet. For example, the chow may contain about 21% milk fat and about 0.15% cholesterol by weight in a diet where 42% of total calories are derived from fat. See, e.g., Harlan Teklad, diet TD88137. When the inhibitor is mixed with the chow, the vehicle, either with or without the inhibitor, can be mixed with the chow and fed to the mice every day for the duration of the study.

Triglyceride measurements can be taken for a duration of about 6 to about 8 weeks, with the subject animals being dosed every day during this period. Typical dosing groups, containing about 6 to about 8 animals per group, can be composed of an untreated control group, a vehicle control group, and dose-treated groups ranging from about 10 mg/kg body weight to about 90 mg/kg body weight. On a weekly basis, plasma samples can be obtained from the subject animals and analyzed for total triglycerides, for example, to determine the amount of lipids absorbed into the blood circulation.

The effect of the non-absorbed phospholipase inhibitor, e.g., a phospholipase A2 inhibitor, is a net decrease in lipid plasma levels, which indicates reduced fat absorption, in the animals of the dose-treated groups fed a Western (high fat/high cholesterol) diet relative to the animals of the control groups. Effective dosages can also be determined.

### Example 9

### Reduction in Diet-Induced Hypercholesterolemia in a Mouse Model

A phospholipase inhibitor, for example a composition comprising a phospholipase inhibiting moiety disclosed herein, can be used in a mouse model to demonstrate, for example, suppression of diet-induced hypercholesterolemia. The phospholipase inhibitor can be administered to subject animals either as a chow supplement and/or by oral gavage BID (e.g., less than about 1 ml/kg body weight, or about 25 to about 50 μl/dose). A typical vehicle for inhibitor suspension comprises about 0.9% carboxymethylcellulose, about 9% PEG-400, and about 0.05% Tween 80, with an inhibitor concentration of about 5 to about 13 mg/ml. This suspension can be added as a supplement to daily chow, e.g., less than about 0.015% of the diet by weight, and/or administered by oral gavage BID, e.g., with a daily dose of about 10 mg/kg to about 90 mg/kg body weight.

The mouse chow used may have a composition representative of a Western-type (high fat and/or high cholesterol) diet. For example, the chow may contain about 21% milk fat and about 0.15% cholesterol by weight in a diet where 42% of total calories are derived from fat. See, e.g., Harlan Teklad, diet TD88137. When the inhibitor is mixed with the chow, the vehicle, either with or without the inhibitor, can be mixed with the chow and fed to the mice every day for the duration of the study.

Cholesterol and/or triglyceride measurements can be taken for a duration of about 6 to about 8 weeks, with the subject animals being dosed every day during this period. Typical dosing groups, containing about 6 to about 8 animals per group, can be composed of a untreated control group, a vehicle control group, and dose-treated groups ranging from about 10 mg/kg body weight to about 90 mg/kg body weight. On a weekly basis, plasma samples can be obtained from the subject animals and analyzed for total cholesterol and/or triglycerides, for example, to determine the amount of cholesterol and/or lipids absorbed into the blood circulation. Since most plasma cholesterol in a mouse is associated with HDL (in contrast to the LDL association of most cholesterol in humans), HDL and non-HDL fractions can be separated to aid determination of the effectiveness of the non-absorbed phospholipase inhibitor in lowering plasma non-HDL levels, for example VLDL/LDL.

The effect of the non-absorbed phospholipase inhibitor, e.g., a phospholipase A2 inhibitor, is a net decrease in hypercholesterolemia in the animals of the dose-treated groups fed a Western (high fat/high cholesterol) diet relative to the animals of the control groups. Effective dosages can also be determined.

### Example 10

### In-Vivo Evaluation of ILY-4001 [2-(3-(2-amino-2-oxoacetyl)-1-(biphenyl-2-ylmethyl)-2-methyl-1H-indol-4-yloxy)acetic acid] as PLA2-IB Inhibitor and for Treatment of Diet-Related Conditions

This example demonstrated that the compound 2-(3-(2-amino-2-oxoacetyl)-1-(biphenyl-2-ylmethyl)-2-methyl-1H-indol-4-yloxy)acetic acid, shown in FIG. 2, was an effective phospholipase-2A IB inhibitor, with phenotypic effects approaching and/or comparable to the effect of genetically deficient PLA2 (−/−) mice. This example also demonstrated that this compound is effective in treating conditions such as weight-related conditions, insulin-related conditions, and cholesterol-related conditions, including in particular conditions such as obesity, diabetes mellitus, insulin resistance, glucose intolerance, hypercholesterolemia and hypertriglyceridemia. In this example, the compound 2-(3-(2-amino-2-oxoacetyl)-1-(biphenyl-2-ylmethyl)-2-methyl-1H-indol-4-yloxy)acetic acid is designated as ILY-4001 (and is alternatively referred to herein as methyl indoxam).

ILY-4001 (FIG. 5) was evaluated as a PLA2 IB inhibitor in a set of experiments using wild-type mice and genetically deficient PLA2 (−/−) mice (also referred to herein as PLA2 knock-out (KO) mice). In these experiments, wild-type and PLA2 (−/−) mice were maintained on a high fat/high sucrose diet, details of which are described below.

ILY-4001 has a measured IC50 value of around 0.2 uM versus the human PLA2 IB enzyme and 0.15 uM versus the mouse PLA2 IB enzyme, in the context of the 1-palmitoyl-2-(10-pyrenedecanoyl)-sn-glycero-3-phosphoglycerol assay, which measures pyrene substrate release from vesicles treated with PLA2 IB enzyme (Singer, Ghomashchi et al. 2002). An IC-50 value of around 0.062 was determined in experimental studies. (See Example 1B-1). In addition to its activity against mouse and human pancreatic PLA2, methyl indoxam is stable at low pH, and as such, would be predicted to survive passage through the stomach. ILY-4001 has relatively low absorbtion from the GI lumen, based on Caco-2 assays (See Example 1B-2), and based on pharmokinetic studies (See Example 1B-3).

In the study of this Example 10, twenty-four mice were studied using treatment groups as shown in Table 5, below. Briefly, four groups were set up, each having six mice. Three of the groups included six wild-type PLA2 (+/+) mice in each group (eighteen mice total), and one of the groups included six genetically deficient PLA2 (−/−) mice. One of the wild-type groups was used as a wild-type control group, and was not treated with ILY-4001. The other two wild-type groups were treated with ILY-4001—one group at a lower dose (indicated as “L” in Table 1) of 25 mg/kg/day, and the other at a higher dose (indicated as “H” in Table 1) of 90 mg/kg/day. The group comprising the PLA2 (−/−) mice was used as a positive control group.

TABLE 5
Treatment Groups for ILY-4001 Study

| | | | ILY-4001 | |
|---|---|---|---|---|
| Group | Treatment | Number | Dose Levels | Durati [symbol] |
| Number | Groups | of Animals | (mg/kg/day) | (week [symbol] |
| 1 | C57BL/6(wt) | 6 | 0 | 10 |
| 2 | C57BL/6(wt) | 6 | 25 (L) | 10 |
| 3 | C57BL/6(wt) | 6 | 90 (H) | 10 |
| 4 | C57BL/6(PLA₂- | 6 | 0 | 10 |
| | KO) | | | |

The experimental protocol used in this study was as follows. The four groups of mice, including wild type and isogenic PLA2 (−/−) C57BL/J mice were acclimated for three days on a low fat/low carbohydrate diet. After the three day acclimation phase, the animals were fasted overnight and serum samples taken to establish baseline plasma cholesterol, triglyceride, and glucose levels, along with baseline body weight. The mice in each of the treatment groups were then fed a high fat/high sucrose diabetogenic diet (Research Diets D12331). 1000 g of the high fat/high sucrose D12331 diet was composed of casein (228 g), DL-methionine (2 g), maltodextrin 10 (170 g), sucrose (175 g), soybean oil (25 g), hydrogenated coconut oil (333.5 g), mineral mix S10001 (40 g), sodium bicarbonate (10.5 g), potassium citrate (4 g), vitamin mix V10001 (10 g), and choline bitartrate (2 g). This diet was supplemented with ILY-4001 treatments such that the average daily dose of the compound ingested by a 25 g mouse was: 0 mg/kg/day (wild-type control group and PLA2 (−/−) control group); 25 mg/kg/day (low-dose wild-type treatment group), or 90 mg/kg/day (high-dose wild-type treatment group). The animals were maintained on the high fat/high sucrose diet, with the designated ILY-4001 supplementation, for a period of ten weeks.

Body weight measurements were taken for all animals in all treatment and control groups at the beginning of the treatment period and at 4 weeks and 10 weeks after initiation of the study. (See Example 10A). Blood draws were also taken at the beginning of the treatment period (baseline) and at 4 weeks and 10 weeks after initiation of the study, in order to determine fasting glucose (See Example 10B). Cholesterol and triglyceride levels were determined from blood draws taken at the beginning of the treatment (baseline) and at ten weeks. (See Example 10C).

### Example 5A

### Body-Weight Gain in In-Vivo Evaluation of ILY-4001 [2-(3-(2-amino-2-oxoacetyl)-1-(biphenyl-2-ylmethyl)-2-methyl-1H-indol-4-yloxy)acetic acid] as PLA2-IB Inhibitor

In the study generally described above in Example 10, body weight measurements were taken for all animals in all treatment and control groups at the beginning of the treatment period and at 4 weeks and 10 weeks after initiation of the study. Using the treatment protocol described above with ILY-4001 supplemented into a high fat/high sucrose diabetogenic diet, notable decreases were seen in body weight gain.

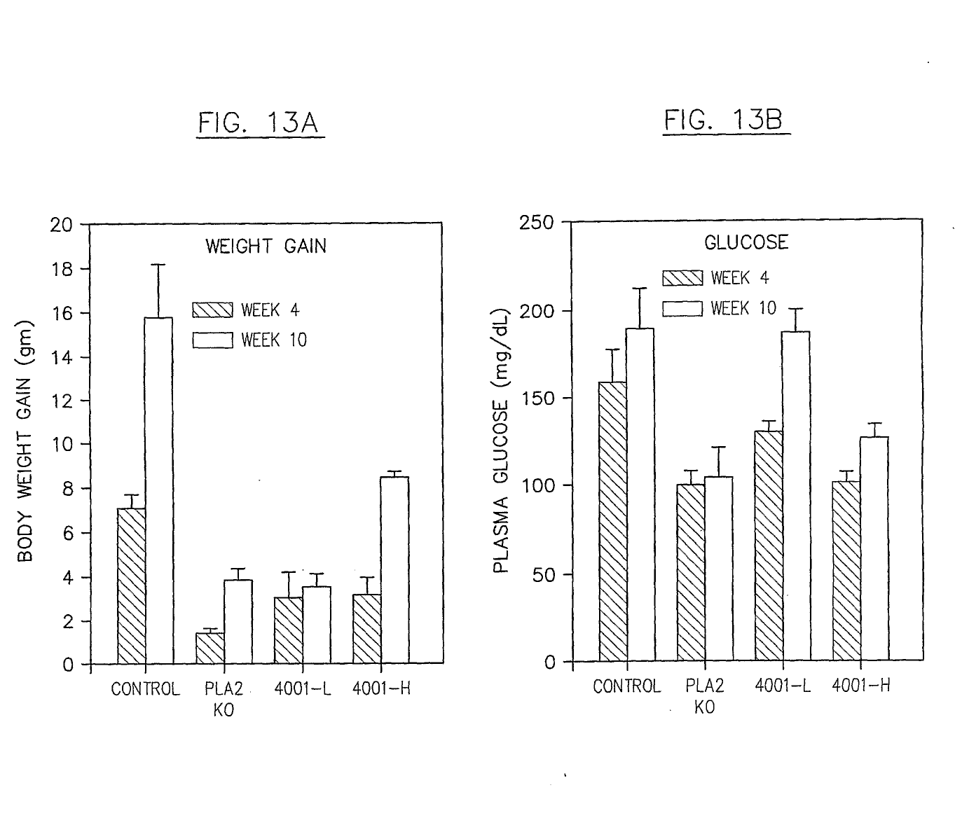

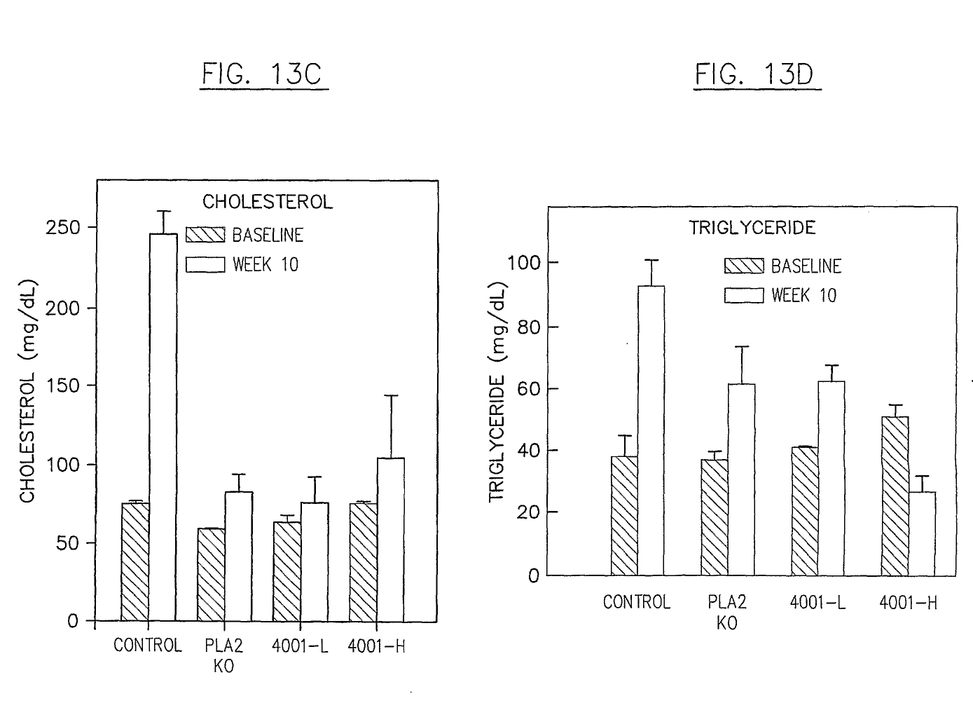

With reference to FIG. 13A, body weight gain in the wild-type mice receiving no ILY-4001 (group 1, wild-type control) followed the anticipated pattern of a substantial weight gain from the beginning of the study to week 4, and a further doubling of weight gain by week 10. In contrast, body weight gain for the PLA2 (−/−) mice (PLA2 KO mice) also receiving no ILY-4001 and placed on the same diet (group 4, PLA2 (−/−) control) did not show statistically significant changes from week 4 to week 10, and only a marginal increase in body weight over the extent of the study (<5g). The two treatment groups (25 mg/kg/d and 90 mg/kg/d) showed significantly reduced body weight gains at week 4 and week 10 of the study compared to the wild-type control group. Both treatment groups showed body weight gain at four weeks modulated to an extent approaching that achieved in the PLA2 (−/−) mice. The low-dose treatment group showed body weight gain at ten weeks modulated to an extent comparable to that achieved in the PLA2 (−/−) mice.

### Example 10B

### Fasting Serum Glucose in In-Vivo Evaluation of ILY-4001 [2-(3-(2-amino-2-oxoacetyl)-1-(biphenyl-2-ylmethyl)-2-methyl-1H-indol-4-yloxy)acetic acid] as PLA2-IB Inhibitor

In the study generally described above in Example 10, blood draws were taken at the beginning of the treatment period (baseline) and at 4 weeks and 10 weeks after initiation of the study, in order to determine fasting glucose. Using the treatment protocol described above with ILY-4001 supplemented into a high fat/high sucrose diabetogenic diet, notable decreases were seen in fasting serum glucose levels.

Referring to FIG. 13B, the wild-type control mice (group 1) showed a sustained elevated plasma glucose level, consistent with and indicative of the high fat/high sucrose diabetogenic diet at both four weeks and ten weeks. In contrast, the PLA2 (−/−) KO mice (group 4) showed a statistically significant decrease in fasting glucose levels at both week 4 and week 10, reflecting an increased sensitivity to insulin not normally seen in mice placed on this diabetogenic diet. The high dose ILY-4001 treatment group (group 3) showed a similar reduction in fasting glucose levels at both four weeks and ten weeks, indicating an improvement in insulin sensitivity for this group as compared to wild-type mice on the high fat/high sucrose diet, and approaching the phenotype seen in the PLA2 (−/−) KO mice. In the low dose ILY-4001 treatment group (group 2), a moderately beneficial effect was seen at week four; however, a beneficial effect was not observed at week ten.

### Example 10C

### Serum Cholesterol and Triglycerides in In-Vivo Evaluation of ILY-4001 [2-(3-(2-amino-2-oxoacetyl)-1-(biphenyl-2-ylmethyl)-2-methyl-1H-indol-4-yloxy)acetic acid] as PLA2-IB Inhibitor

In the study generally described above in Example 10, blood draws were taken at the beginning of the treatment period (baseline) and at 10 weeks after initiation of the study, in order to determine cholesterol and triglyceride levels. Using the treatment protocol described above with ILY-4001 supplemented into a high fat/high sucrose diabetogenic diet, notable decreases were seen in both serum cholesterol levels and serum triglyceride levels.

With reference to FIGS. 13C and 13D, after 10 weeks on the high fat/high sucrose diet, the wild-type control animals (group 1) had notable and substantial increases in both circulating cholesterol levels (FIG. 13C) and triglyceride levels (FIG. 13D), relative to the baseline measure taken at the beginning of the study. The PLA2 (−/−) KO animals (group 4), in contrast, did not show the same increase in these lipids, with cholesterol and triglyceride values each 2 to 3 times lower than those found in the wild-type control group. Significantly, treatment with ILY-4001 at both the low and high doses (groups 2 and 3, respectively) substantially reduced the plasma levels of cholesterol and triglycerides, mimicking the beneficial effects at levels comparable to the PLA2 (−/−) KO mice.

All publications, patents, and patent applications mentioned in this specification are herein incorporated by reference to the same extent as if each individual publication, patent, or patent application was specifically and individually indicated to be incorporated by reference.

It can be appreciated to one of ordinary skill in the art that many changes and modifications can be made thereto without departing from the spirit or scope of the appended claims, and such changes and modifications are contemplated within the scope of the instant invention.

1.-188. (canceled)

189. A composition comprising a phospholipase inhibitor, the phospholipase inhibitor comprising an oligomer or polymer moiety covalently linked to a phospholipase inhibiting moiety, the phospholipase inhibitor having the formula 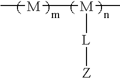
wherein n is a non-zero integer, m is an integer, M is a monomer moiety, L is a linking moiety and Z is a phospholipase inhibiting moiety.

190. A composition comprising a phospholipase inhibitor, the phospholipase inhibitor comprising an oligomer or polymer moiety covalently linked to a phospholipase inhibiting moiety, the phospholipase inhibitor having the formula 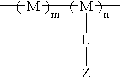
wherein n is a non-zero integer, m is an integer, M is a monomer moiety, L is a linking moiety and Z is a phospholipase inhibiting moiety, the phospholipase inhibitor being characterized by the phospholipase inhibitor inhibiting activity of a phosholipase-A2 IB and by the phospholipase inhibitor being localized in the gastrointestinal lumen, such that upon administration to the subject, essentially all of the phospholipase inhibitor remains in the gastrointestinal lumen.

191. A composition comprising a phospholipase inhibitor, the phospholipase inhibitor comprising an oligomer or polymer moiety covalently linked to a phospholipase inhibiting moiety, the phospholipase inhibitor having the formula 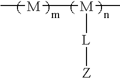
wherein n is a non-zero integer, m is an integer, M is a monomer moiety, L is a linking moiety and Z is a phospholipase inhibiting moiety, the phospholipase inhibitor being characterized by one or more features selected from the group consisting of:
(a) the phospholipase inhibitor being stable while passing through at least the stomach, the duodenum and the small intestine of the gastrointestinal tract;
(b) the phospholipase inhibitor inhibiting activity of a secreted, calcium-dependent phospholipase present in the gastrointestinal lumen;
(c) the phospholipase inhibitor inhibiting activity of a phosholipase-A2, but essentially not inhibiting other gastrointestinal mucosal membrane-bound phospholipases;
(d) the phospholipase inhibitor being insoluble in the fluid phase of the gastrointestinal tract;
(e) the phospholipase inhibitor being adapted to associate with a lipid-water interface;
(f) the oligomer or polymer moiety comprising at least one monomer moiety that is anionic and at least one monomer moiety that is hydrophobic;
(g) the oligomer or polymer moiety being a copolymer moiety, the copolymer moiety being a random copolymer moiety, a block copolymer moiety; a grafted copolymer moiety; a hydrophobic copolymer moiety; and combinations thereof; and
(h) combinations thereof, including each permutation of combinations.

192. A composition comprising a phospholipase inhibitor, the phospholipase inhibitor comprising an oligomer or polymer moiety covalently linked to a phospholipase inhibiting moiety, the phospholipase inhibitor having the formula 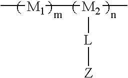
wherein m is a non-zero integer, n is a non-zero integer, M1 is a first monomer moiety, M2 is a second monomer moiety, the second monomer moiety being the same as or different than the first monomer moiety, L is an optional linking moiety and Z is a phospholipase inhibiting moiety, the phospholipase inhibitor being characterized by the phospholipase inhibitor inhibiting activity of a phosholipase-A2 IB and by the phospholipase inhibitor being localized in the gastrointestinal lumen, such that upon administration to the subject, essentially all of the phospholipase inhibitor remains in the gastrointestinal lumen.

193. The composition of any of claims 189 through 192 wherein the phospholipase inhibitor is localized in the gastrointestinal lumen such that upon administration to the subject, at least about 80% of the phospholipase inhibitor remains in the gastrointestinal lumen.

194. The composition of any of claims 189 through 192 wherein n and m, independently, range from about 1 to about 2000.

195. The composition of any of claims 189 through 192 wherein n and m, independently, range from about 1 to about 500.

196. The composition of any of claims 189 through 192 wherein the molecular weight of the oligomer or polymer moiety ranges from about 1000 Daltons to about 500,000 Daltons.

197. The composition of any of claims 189 through 192 wherein the molecular weight of the oligomer or polymer moiety ranges from about 5000 Daltons to about 200,000 Daltons.

198. The composition of any of claims 189 through 192 wherein the length of L ranges from about 1 atom to about 10 atoms.

199. The composition of any of claims 189 through 192 wherein the number of phospholipase inhibiting moieties, Z, ranges from about 1 to about 2000.

200. The composition of any of claims 189 through 192 wherein the number of phospholipase inhibiting moieties, Z, ranges from about 1 to about 500.

201. The composition of any of claims 189 through 192 wherein the phospholipase inhibitor comprises at least one moiety selected from a hydrophobic moiety, a hydrophilic moiety, a charged moiety and combinations thereof.

202. The composition of any of claims 189, 190, or 192 wherein the phospholipase inhibitor moiety is soluble.

203. The composition of any of claims 189 through 192 wherein the phospholipase inhibitor moiety is insoluble.

204. The composition of any of claims 189, 190, or 192 wherein the phospholipase inhibitor comprises an anionic or hydrophilic polymer moiety and scavenges a phospholipase in a gastrointestinal lumen fluid.

205. The composition of any of claims 189 through 192 wherein the phospholipase inhibitor comprises a hydrophobic polymer moiety and associates with a lipid-water interface.

206. The composition of any of claims 189 through 192 wherein the phospholipase inhibitor comprises a copolymer moiety comprising at least one anionic monomer moiety and at least one hydrophobic monomer moiety, and wherein the phospholipase inhibitor interacts with a phospholipase and with a lipid-water interface.

207. The composition of any of claims 189 or 190 wherein the phospholipase inhibitor comprises a homopolymer moiety.

208. The composition of any of claims 189 through 191 wherein the phospholipase inhibitor comprises a copolymer moiety.

209. The composition of any of claims 189 through 191 wherein the oligomer or polymer moiety is a cross-linked oligomer or polymer moiety.

210. The composition of any of claims 189 through 191 wherein the oligomer or polymer moiety comprises a hydrophobic monomer moiety.

211. The composition of any of claims 189 through 191 wherein the oligomer or polymer comprises an anionic monomer moiety.

212. The composition of any of claims 189 or 190 wherein the oligomer or polymer moiety comprises a hydrophilic monomer moiety.

213. The composition of any of claims 189 through 192 wherein each M is independently selected from a first monomer moiety, M1, a second monomer moiety, M2, different from the first monomer moiety, and combinations thereof.

214. The composition of claim 192 wherein the monomer moieties M1, or M2, independently, comprises at least one moiety selected from an acrylic moiety, a methacrylic moiety, a vinylic moiety, an allylic moiety and a styrenic moiety.

215. The composition of claims 192 wherein M1 and M2 are the same, whereby the phospholipase inhibitor comprises a homopolymer oligomer or polymer moiety.

216. The composition of claim 192 wherein M1 and M2 are different, whereby the phospholipase inhibitor comprises a copolymer oligomer or polymer moiety.

217. The composition of claim 192 wherein M1 and M2 are different, the phospholipase inhibitor comprising a random copolymer oligomer or polymer moiety.

218. The composition of claim 192 wherein M1 and M2 are different, the phospholipase inhibitor comprising a block copolymer oligomer or polymer moiety.

219. The composition of claim 192 wherein said oligomer or polymer moiety is at least one selected from a carboxymethylcellulose, a chitosan, and a sulfoethylcellulose oligomer or polymer moiety.

220. The composition of claim 192 wherein said oligomer or polymer moiety is a graft oligomer or polymer moiety or a hyperbranched oligomer or polymer moiety.

221. The composition of claim 192 wherein the phospholipase inhibiting moiety is a small molecule.

222. The composition of claim 192 wherein the phospholipase inhibiting moiety is at least one compound selected from an arachidonic acid analogue; an arachidonyl trifluoromethyl ketone; a methylarachidonyl fluorophosphonate; a palmitoyl trifluoromethyl ketone; a benzensulfonamide derivative, a bromoenol lactone, a p-bromophenyl bromide, a bromophenacyl bromide, a trifluoromethylketone, a sialoglycolipid and a proteoglycan.

223. The composition of claim 192 wherein the phospholipase inhibiting moiety is a phospholipid analog or a transition state analog.

224. The composition of claim 223 wherein the phospholipid analog or the transition state analog is linked to the oligomer or polymer moiety via a hydrophobic group of the phospholipid analog or of the transition state analog.

225. The composition of claim 192 wherein the phospholipase inhibiting moiety comprises a substituted organic compound having a fused five-member ring and six-member ring.

226. The composition of claim 225 wherein the phospholipase inhibiting moiety comprises a fused five-member ring and six-member ring having one or more heteroatoms substituted within the ring structure of the five-member ring, within the ring structure of the six-member ring, or within the ring structure of each of the five-member and six-member rings.

227. The composition of claim 192 wherein the phospholipase inhibiting moiety comprises an indole moiety.

228. The composition of claim 192 wherein the phospholipase inhibiting moiety comprises a compound, or a salt thereof represented by the formula 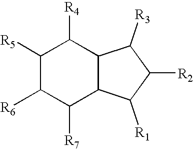
wherein the fused five-membered-ring and six-membered-ring core structure can be saturated or unsaturated, and wherein R1 through R7 are independently selected from the group consisting of: hydrogen, oxygen, sulfur, phosphorus, amine, halide, hydroxyl (—OH), thiol (—SH), carbonyl, acidic, alkyl, alkenyl, carbocyclic, heterocyclic, acylamino, oximyl, hydrazyl, substituted substitution group, and combinations thereof.

229. The composition of claim 228 wherein R1 through R7 can independently comprise, independently selected additional rings between two adjacent substitutents, such additional rings being independently selected 5-, 6-, and/or 7-member rings which are carbocyclic rings, heterocyclic rings, and combinations thereof.

230. The composition of claim 192 wherein the phospholipase inhibitor comprises an indole compound, or a salt thereof, selected from the formulas 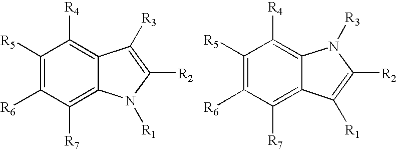
wherein with respect to each of the formulas, R1 through R7 are independently selected from the groups consisting of: hydrogen, oxygen, sulfur, phosphorus, amine, halide, hydroxyl (—OH), thiol (—SH), carbonyl, acidic, alkyl, alkenyl, carbocyclic, heterocyclic, acylamino, oximyl, hydrazyl, substituted substitution group, and combinations thereof.

231. The composition of claim 230 wherein with respect to each of the formulas, R1 through R7 can independently comprise, independently selected additional rings between two adjacent substitutents, such additional rings being independently selected 5-, 6-, and/or 7-member rings which are carbocyclic rings, heterocyclic rings, and combinations thereof.

232. The composition of claim 230 wherein
R1 is selected from the group consisting of hydrogen, oxygen, sulfur, amine, halide, hydroxyl (—OH), thiol (—SH), carbonyl, acidic, alkyl, alkenyl, carbocyclic, heterocyclic, and substituted substitution group;
R2 is selected from the group consisting of hydrogen, oxygen, halide, carbonyl, alkyl, alkenyl, carbocyclic, and substituted substitution group;
R3 is selected from the group consisting of hydrogen, oxygen, sulfur, amine, hydroxyl (—OH), thiol (—SH), carbonyl, acidic, alkyl, heterocyclic, acylamino, oximyl, hydrazyl, and substituted substitution group;
R4 and R5 are each independently selected from the group consisting of hydrogen, oxygen, sulfur, phosphorus, amine, hydroxyl (—OH), thiol (—SH), carbonyl, acidic, alkyl, alkenyl, heterocyclic, acylamino, oximyl, hydrazyl, and substituted substitution group;
R6 is selected from the group consisting of hydrogen, oxygen, amine, halide, hydroxyl (—OH), acidic, alkyl, carbocyclic, acylamino and substituted substitution group; and
R7 is selected from the groups consisting of hydrogen, halide, thiol (—SH), carbonyl, acidic, alkyl, alkenyl, carbocyclic, and substituted substitution group.

233. The composition of claim 232 wherein R1 is selected from the group consisting of alkyl, carbocyclic and substituted substitution group.

234. The composition of claim 232 wherein R2 is selected from the group consisting of halide, alkyl and substituted substitution group.

235. The composition of claim 232 wherein R3 is selected from the group consisting of carbonyl, acylamino, oximyl, hydrazyl, and substituted substitution group.

236. The composition of claim 232 wherein R4 and R5 are each independently selected from the group consisting of oxygen, hydroxyl (—OH), acidic, alkyl, and substituted substitution group.

237. The composition of claim 232 wherein R6 is selected from the group consisting of amine, acidic, alkyl, and substituted substitution group.

238. The composition of claim 232 wherein R7 is selected from the groups consisting of carbocyclic and substituted substitution group.

239. The composition of claim 192 wherein the phospholipase inhibiting moiety is a compound or a salt thereof having the formula

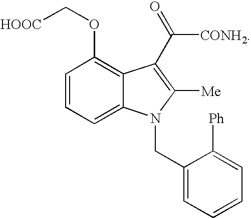

240. The composition of claim 189 wherein the phospholipase inhibitor has a permeability coefficient lower than about −5.

241. The composition of claim 189 wherein the phospholipase inhibitor inhibits phospholipase B.

242. The composition of claim 189 wherein the phospholipase inhibitor essentially does not inhibit a lipase.

243. The composition of claim 189 wherein the phospholipase inhibitor essentially does not inhibit phospholipase-B.

244. The composition of claim 189 wherein the phospholipase inhibitor inhibits activity of phospholipase A2 IB, but essentially does not inhibit other gastrointestinal phospholipases having activity for catabolizing a phospholipid.

245. The composition of claim 189 wherein the phospholipase inhibitor inhibits activity of phospholipase A2 IB, but essentially does not inhibit other gastrointestinal phospholipases having activity for catabolizing phosphatidylcholine or phosphatidylethanolamine.

246. The composition of claim 189 wherein the phospholipase inhibitor inhibits activity of phospholipase A2 IB, but essentially does not inhibit other gastrointestinal mucosal membrane-bound phospholipases.

247. The composition of claim 189 wherein said inhibitor produces a therapeutic or prophylatic benefit in treating an insulin-related condition in a subject receiving said inhibitor.

248. The composition of claim 189 wherein said inhibitor produces a therapeutic or prophylactic benefit in treating a weight-related condition in a subject receiving said inhibitor.

249. The composition of claim 189 wherein said inhibitor produces a therapeutic or prophylactic benefit in treating a cholesterol-related condition in a subject receiving said inhibitor.
